(19) United States
(12) Patent Application Publication
(10) Pub. No.: US 2021/0388424 A1
(43) Pub. Date: Dec. 16, 2021

(54) METHODS FOR ANALYZING TARGET NUCLEIC ACIDS AND RELATED COMPOSITIONS

(71) Applicant: 10x Genomics, Inc., Pleasanton, CA (US)

(72) Inventor: Felice Alessio BAVA, Pleasanton, CA (US)

(73) Assignee: 10x Genomics, Inc., Pleasanton, CA (US)

(21) Appl. No.: 17/346,079

(22) Filed: Jun. 11, 2021

Related U.S. Application Data
(60) Provisional application No. 63/038,637, filed on Jun. 12, 2020.

(51) Int. Cl. C12Q 1/6841; C12Q 1/6874

(52) U.S. Cl. CPC C12Q 1/6841; C12Q 1/6874

(57) ABSTRACT

The present disclosure, among other things, provides methods for analyzing a target nucleic acid in a biological sample. In some aspects, the methods involve the use of a set of probe polynucleotides, for example comprising three polynucleotides, for assessing target nucleic acids. In some aspects, the presence, amount and/or sequence of the target nucleic acid is analyzed in situ. In some aspects, the methods involve anchoring or linking a portion of the set of polynucleotides to a scaffold or other nucleic acids. Also provided are polynucleotides, set of polynucleotides, compositions, kits, devices and systems for use in accordance with the methods.

### CROSS REFERENCE TO RELATED APPLICATIONS

This application claims priority benefit of U.S. Provisional Application No. 63/038,637, filed Jun. 12, 2020, entitled “METHODS FOR ANALYZING TARGET NUCLEIC ACIDS AND RELATED COMPOSITIONS,” the contents of which are incorporated herein by reference in their entireties for all purposes.

### FIELD

The present disclosure relates in some aspects to methods for analyzing a target nucleic acid in a biological sample. In some aspects, the methods involve the use of a set of probe polynucleotides, for example comprising three polynucleotides, for assessing target nucleic acids. In some aspects, the presence, amount and/or sequence of the target nucleic acid is analyzed in situ. In some aspects, the methods involve anchoring or linking a portion of the set of polynucleotides to a scaffold or other nucleic acids. Also provided are polynucleotides, set of polynucleotides, compositions, kits, devices and systems for use in accordance with the methods.

### BACKGROUND

Methods are available for analyzing nucleic acids present in a biological sample, such as a cell or a tissue. Current methods for analyzing nucleic acids present in a biological sample, for example for in situ analysis, can have low sensitivity and specificity, limited plexity, biased, time-consuming, labor-intensive, and/or error-prone. Improved methods for analyzing nucleic acids present in a biological sample are needed. Provided herein are methods, polynucleotides, set of polynucleotides, compositions, kits, devices and systems, that meet such and other needs.

### SUMMARY

Provided herein are methods involving the use of a set of polynucleotides, such as one or more polynucleotide(s), for analyzing one or more target nucleic acid(s). In some of any of the provided embodiments, the target nucleic acid is analyzed using a polynucleotide probe set that comprises a first polynucleotide, a second polynucleotide, and a third polynucleotide. In some of any of the provided embodiments, the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3. In some of any of the provided embodiments, the target nucleic acid comprises, in the 3′ to 5′ direction, hybridization regions HR1′, HR2′, and HR3′. In some of any of the provided embodiments, HR1, HR2, and HR3 hybridize to HR1′, HR2′, and HR3′, respectively; HRa′ hybridizes to HRa; and HRb1 and HRb2 form a split hybridization region that hybridizes to HRb′. In some embodiments, the second polynucleotide is circularized. In some embodiments, the second polynucleotide is amplified. In some embodiments, a sequence in the amplification product is analyzed.

Also provided herein are methods for analyzing a target nucleic acid that involves contacting a target nucleic acid with a first polynucleotide, a second polynucleotide, and a third polynucleotide to form a hybridization complex, wherein: the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3; the target nucleic acid comprises, in the 3′ to 5′ direction, hybridization regions HR1′, HR2′, and HR3′; HR1, HR2, and HR3 hybridize to HR1′, HR2′, and HR3′, respectively; HRa′ hybridizes to HRa; and HRb1 and HRb2 form a split hybridization region that hybridizes to HRb′; wherein HRb1 and HRb2 are connected using HRb′ as a splint to circularize the second polynucleotide; wherein an amplification product is formed using the circularized second polynucleotide as a template and the first polynucleotide as a primer; and wherein a sequence in the amplification product is analyzed, and the sequence is indicative of the target nucleic acid or a sequence thereof.

Also provided herein are methods for analyzing a target nucleic acid that involves circularizing a second polynucleotide in a hybridization complex comprising a target nucleic acid, a first polynucleotide, the second polynucleotide, and a third polynucleotide, wherein: the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3; the target nucleic acid comprises, in the 3′ to 5′ direction, hybridization regions HR1′, HR2′, and HR3′; HR1, HR2, and HR3 hybridize to HR1′, HR2′, and HR3′, respectively; HRa′ hybridizes to HRa; HRb1 and HRb2 form a split hybridization region that hybridizes to HRb′, and the second polynucleotide is circularized by connecting HRb1 and HRb2 using HRb′ as a splint; wherein an amplification product is formed using the circularized second polynucleotide as a template and the first polynucleotide as a primer; and wherein a sequence in the amplification product is analyzed, and the sequence is indicative of the target nucleic acid or a sequence thereof.

Also provided herein are methods for analyzing a target nucleic acid that involves forming an amplification product using a circularized second polynucleotide as a template and a first polynucleotide as a primer, wherein: a target nucleic acid, the first polynucleotide, the circularized second polynucleotide, and a third polynucleotide form a hybridization complex; the first polynucleotide comprises, in the 5′ to 3′ direction, hybridization regions HR1 and HRa′; prior to circularization, the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3; the target nucleic acid comprises, in the 3′ to 5′ direction, hybridization regions HR1′, HR2′, and HR3′; HR1, HR2, and HR3 hybridize to HR1′, HR2′, and HR3′, respectively; HRa′ hybridizes to HRa; prior to circularization, HRb1 and HRb2 form a split hybridization region that hybridizes to HRb′, and the second polynucleotide is circularized by connecting HRb1 and HRb2 using HRb′ as a splint; and wherein a sequence in the amplification product is analyzed, and the sequence is indicative of the target nucleic acid or a sequence thereof.

Also provided herein are methods for analyzing a target nucleic acid that involves analyzing a sequence in an amplification product, wherein the sequence is indicative of a target nucleic acid or a sequence thereof, wherein the amplification product is formed using a circularized second polynucleotide as a template and a first polynucleotide as a primer, and wherein: a target nucleic acid, the first polynucleotide, the circularized second polynucleotide, and a third polynucleotide form a hybridization complex; the first polynucleotide comprises, in the 5′ to 3′ direction, hybridization regions HR1 and HRa′; prior to circularization, the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3; the target nucleic acid comprises, in the 3′ to 5′ direction, hybridization regions HR1′, HR2′, and HR3′; HR1, HR2, and HR3 hybridize to HR1′, HR2′, and HR3′, respectively; HRa′ hybridizes to HRa; prior to circularization, HRb1 and HRb2 form a split hybridization region that hybridizes to HRb′, and the second polynucleotide is circularized by connecting HRb1 and HRb2 using HRb′ as a splint.

Also provided are methods for analyzing a target nucleic acid that involves contacting a target nucleic acid with a first polynucleotide, a second polynucleotide, and a third polynucleotide to form a hybridization complex, wherein: the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3; the target nucleic acid comprises, in the 3′ to 5′ direction, hybridization regions HR1′, HR2′, and HR3′; HR1, HR2, and HR3 hybridize to HR1′, HR2′, and HR3′, respectively; HRa′ hybridizes to HRa; and HRb1 and HRb2 form a split hybridization region that hybridizes to HRb′; circularizing the second polynucleotide by connecting HRb1 and HRb2 using HRb′ as a splint; forming an amplification product using the circularized second polynucleotide as a template and the first polynucleotide as a primer; and analyzing a sequence in an amplification product, wherein the sequence is indicative of a target nucleic acid or a sequence thereof.

In some of any embodiments, HR1′ is between about 4 and about 16 nucleotides in length. In some of any embodiments, HR1′ is between about 8 and about 12 nucleotides in length, optionally about 10 nucleotides in length. In some of any embodiments, HR2′ is between about 4 and about 16 nucleotides in length. In some of any embodiments, HR2′ is between about 8 and about 12 nucleotides in length, optionally about 10 nucleotides in length. In some of any embodiments, HR3′ is between about 4 and about 16 nucleotides in length. In some of any embodiments, HR3′ is between about 8 and about 12 nucleotides in length, optionally about 10 nucleotides in length, optionally wherein each of HR1′, HR2′, and HR3′ is about 10 nucleotides in length. In some of any embodiments, HR1′ and HR2′ are separated by 0 to 2 nucleotides. In some of any embodiments, HR2′ and HR3′ are separated by 0 to 2 nucleotides. In some of any embodiments, HR1 is between about 4 and about 16 nucleotides in length. In some of any embodiments, HR1 is between about 8 and about 12 nucleotides in length, optionally about 10 nucleotides in length. In some of any embodiments, HRa′ is between about 2 and about 16 nucleotides in length. In some of any embodiments, HRa′ is between about 4 and about 8 nucleotides in length, optionally about 6 nucleotides in length. In some of any embodiments, HR1 and HRa′ are separated by 0 to 2 nucleotides. In some of any embodiments, the first polynucleotide is between about 5 and about 40 nucleotides in length.

In some of any embodiments, the first polynucleotide is between about 10 and about 20 nucleotides in length.

In some of any embodiments, HR2 is between about 4 and about 16 nucleotides in length. In some of any embodiments, HR2 is between about 8 and about 12 nucleotides in length, optionally about 10 nucleotides in length. In some of any embodiments, HRa is between about 2 and about 16 nucleotides in length. In some of any embodiments, HRa is between about 4 and about 8 nucleotides in length, optionally about 6 nucleotides in length. In some of any embodiments, HRb1 is between about 1 and about 15 nucleotides in length. In some of any embodiments, HRb2 is between about 1 and about 15 nucleotides in length. In some of any embodiments, HRb1 and HRb2 combined is between about 4 and about 16 nucleotides in length. In some of any embodiments, HRb1 and HRb2 combined is between about 8 and about 12 nucleotides in length, optionally about 10 nucleotides in length. In some of any embodiments, HRa and HR2 are separated by 0 to 2 nucleotides. In some of any embodiments, HR2 and HRb2 are separated by 0 to 2 nucleotides.

In some of any embodiments, the second polynucleotide prior to circularization is between about 15 and about 200 nucleotides in length. In some of any embodiments, the second polynucleotide prior to circularization is between about 25 and about 45 nucleotides in length, optionally about 35 nucleotides in length.

In some of any embodiments, HR3 is between about 4 and about 16 nucleotides in length. In some of any embodiments, HR3 is between about 8 and about 12 nucleotides in length, optionally about 10 nucleotides in length. In some of any embodiments, HRb′ is between about 4 and about 16 nucleotides in length. In some of any embodiments, HRb′ is between about 8 and about 12 nucleotides in length, optionally about 10 nucleotides in length. In some of any embodiments, HRb′ and HR3 are separated by 0 to 2 nucleotides.

In some of any embodiments, the third polynucleotide is between about 5 and about 40 nucleotides in length. In some of any embodiments, the third polynucleotide is between about 10 and about 20 nucleotides in length.

In some of any embodiments, the split hybridization region formed by HRb1 and HRb2 comprises a nick between the 5′ end of HRb1 and the 3′ end of HRb2 when the split hybridization region is hybridized to HRb′.

In some of any embodiments, the methods also involve, without gap filling, ligating HRb1 and HRb2 using HRb′ as a splint.

In some of any embodiments, the split hybridization region formed by HRb1 and HRb2 comprises a gap between the 5′ end of HRb1 and the 3′ end of HRb2 when the split hybridization region is hybridized to HRb′. In some of any embodiments, the gap is between about 1 and about 5 nucleotides in length. In some of any embodiments, the methods also involve, filling the gap and ligating HRb1 and HRb2 using HRb′ as a splint.

In some of any embodiments, the first polynucleotide is a DNA molecule. In some of any embodiments, the second polynucleotide is a DNA molecule. In some of any embodiments, the third polynucleotide is a DNA molecule.

In some of any embodiments, the target nucleic acid comprises an RNA sequence. In some of any embodiments, the target nucleic acid is an mRNA.

In some of any embodiments, the second polynucleotide comprises one or more barcode sequences BC1, BC2, . . . , and BCn, wherein n is an integer of 1 or greater. In some of any embodiments, the first polynucleotide comprises one or more barcode sequences BCα1, BCα2, . . . , and BCαp, wherein p is an integer of 1 or greater. In some of any embodiments, the third polynucleotide comprises one or more barcode sequences BCa1, BCa2, . . . , and BCaq, wherein q is an integer of 1 or greater.

In some of any embodiments, at least one of BC1, BC2, . . . , and BCn, at least one of BCα1, BCα2, . . . , and BCαp, and/or at least one of BCa1, BCa2, . . . , and BCaq identifies the target nucleic acid or a sequence thereof. In some of any embodiments, at least one of BC1, BC2, . . . , and BCn, at least one of BCα1, BCα2, . . . , and BCαp, and/or at least one of BCa1, BCa2, . . . , and BCaq is a unique identifier of a gene. In some of any embodiments, at least one of BC1, BC2, . . . , and BCn, at least one of BCα1, BCα2, . . . , and BCαp, and/or at least one of BCa1, BCa2, . . . , and BCaq is an error-checking barcode. In some of any embodiments, the target nucleic acid is an mRNA and at least one of BC1, BC2, . . . , and BCn, at least one of BCα1, BCα2, . . . , and BCαp, and/or at least one of BCa1, BCa2, . . . , and BCaq identifies the mRNA as a splice variant, a transcriptional variant, and/or identify a splice junction sequence.

In some of any embodiments, the barcode sequence or sequences are between about 6 and about 16 nucleotides in length. In some of any embodiments, the barcode sequence or sequences are between about 8 and about 10 nucleotides in length.

In some of any embodiments, the first polynucleotide, the second polynucleotide, and/or the third polynucleotide comprises an identifying sequence that identifies the target nucleic acid or a sequence thereof, wherein the identifying sequence is between about 3 and about 6 nucleotides in length, optionally about 4 nucleotides in length. In some of any embodiments, the identifying sequence is in HRa, HRb1, and/or HRb2.

In some of any embodiments, the melting temperature (Tₘ) of HR1/HR1′ hybridization, the Tₘ of HR2/HR2′ hybridization, and the Tₘ of HR3/HR3′ hybridization are substantially the same. In some of any embodiments, the melting temperature (Tₘ) of HR1/HR1′ hybridization, the Tₘ of HR2/HR2′ hybridization, and/or the Tₘ of HR3/HR3′ hybridization are between about 40° C. and about 70° C., optionally about 60° C. In some of any embodiments, the melting temperature (Tₘ) of HRa/HRa′ hybridization and/or the Tₘ of HRb1-HRb2/HRb′ hybridization are lower than the Tₘ of HR1/HR1′ hybridization, the Tₘ of HR2/HR2′ hybridization, and/or the Tₘ of HR3/HR3′ hybridization. In some of any embodiments, the melting temperature (Tₘ) of HRa/HRa′ hybridization and/or the Tₘ of HRb1-HRb2/HRb′ hybridization are lower than about 40° C. or is similar to or lower than room temperature.

In some of any embodiments, the hybridization complex is formed at a temperature higher than the melting temperature (Tₘ) of HRa/HRa′ hybridization and/or the Tₘ of HRb1-HRb2/HRb′ hybridization, but lower than the Tₘ of HR1/HR1′ hybridization, the Tₘ of HR2/HR2′ hybridization, and/or the Tₘ of HR3/HR3′ hybridization. In some of any embodiments, the hybridization complex is formed at a temperature between about 30° C. and about 50° C., optionally about 40° C.

In some of any embodiments, the methods also involve, a step of removing molecules that are not specifically hybridized to the target nucleic acid. In some of any embodiments, the removing step comprises a wash, optionally a stringency wash.

In some of any embodiments, the circularization of the second polynucleotide comprises a ligation reaction selected from the group consisting of enzymatic ligation, chemical ligation, optionally click chemistry ligation, template dependent ligation, and/or template independent ligation. In some of any embodiments, the enzymatic ligation utilizes a ligase, optionally a ligase having a DNA-splinted DNA ligase activity, such as a T4 DNA ligase.

In some of any embodiments, the ligation reaction is performed at a temperature lower than the temperature at which the hybridization complex is formed. In some of any embodiments, the ligation reaction is performed at a temperature lower than or similar to the melting temperature (Tₘ) of HRa/HRa′ hybridization and/or the Tₘ of HRb1-HRb2/HRb′ hybridization. In some of any embodiments, the ligation reaction is performed at a temperature between about 10° C. and about 30° C., optionally between about 15° C. and about 25° C.

In some of any embodiments, the methods also involve, a step of removing molecules that are not specifically hybridized in the hybridization complex after the ligation reaction. In some of any embodiments, the removing step comprises a wash, optionally a stringency wash.

In some of any embodiments, the amplification product is formed using isothermal amplification or non-isothermal amplification.

In some of any embodiments, the amplification product is formed using rolling circle amplification (RCA). In some of any embodiments, the RCA comprises a linear RCA, a branched RCA, a dendritic RCA, or any combination thereof. In some of any embodiments, the amplification product is formed using a Phi29 polymerase.

In some of any embodiments, the amplification is performed at a temperature lower than the melting temperature (Tₘ) of HR1/HR1′ hybridization, the Tₘ of HR2/HR2′ hybridization, and/or the Tₘ of HR3/HR3′ hybridization. In some of any embodiments, the amplification is performed at a temperature permissive to HRa/HRa′ hybridization and primer extension by a polymerase, optionally a Phi29 polymerase. In some of any embodiments, the amplification is performed at a temperature between about 15° C. and about 35° C., optionally about 30° C.

In some of any embodiments, the target nucleic acid is analyzed in situ in a tissue sample, optionally a tissue section; optionally wherein the target nucleic acid is an mRNA in a tissue sample, and the analyzing the sequence is performed when the target nucleic acid and/or the amplification product is in situ in the tissue sample.

In some of any embodiments, the tissue sample is an intact tissue sample or a non-homogenized tissue sample. In some of any embodiments, the target nucleic acid is in a cell in the tissue sample.

In some of any embodiments, the methods also involve, permeabilizing and/or fixing the cell or the tissue sample. In some of any embodiments, the tissue sample is a fixed tissue sample, optionally a formalin-fixed, paraffin-embedded (FFPE) sample, a frozen tissue sample, or a fresh tissue sample.

In some of any embodiments, the tissue sample is embedded in a matrix, optionally a hydrogel. In some of any embodiments, the methods also involve, cross-linking the target nucleic acid and/or the amplification product to the matrix.

In some of any embodiments, the analyzing the sequence comprises sequencing all or a portion of the amplification product and/or in situ hybridization to the amplification product. In some of any embodiments, the sequencing comprises sequencing hybridization, sequencing by ligation, and/or fluorescent in situ sequencing, and/or wherein the in situ hybridization comprises sequential fluorescent in situ hybridization.

In some of any embodiments, the analyzing the sequence comprises hybridizing to the amplification product a detection oligonucleotide labeled with a fluorophore, an isotope, a mass tag, or a combination thereof. In some of any embodiments, the analyzing the sequence comprises imaging the amplification product.

In some of any embodiments, the first polynucleotide, the second polynucleotide, and/or the third polynucleotide comprise an anchoring site and/or a hybridization site; optionally wherein the anchoring site comprises a functional group, optionally in a modified nucleotide in the first polynucleotide, the second polynucleotide, and/or the third polynucleotide, that is capable of reacting with a matrix, such as a hydrogel.

Provided herein are polynucleotides and sets of polynucleotides for use in accordance with the methods described herein.

Also provided herein are kits that comprise one or more of the polynucleotides or a set of polynucleotides described herein. Provided in some aspects are kits that include any first polynucleotide, second polynucleotide, and third polynucleotide as described herein.

In some aspects, provided herein are kits comprising a first polynucleotide, a second polynucleotide, and a third polynucleotide, wherein: the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3, and the first polynucleotide, the second polynucleotide, and the third polynucleotide are capable of forming a hybridization complex with a target nucleic acid which comprises, in the 3′ to 5′ direction, hybridization regions HR1′, HR2′, and HR3′, wherein in the hybridization complex, HR1, HR2, and HR3 hybridize to HR1′, HR2′, and HR3′, respectively, HRa′ hybridizes to HRa; and HRb1 and HRb2 form a split hybridization region that hybridizes to HRb′.

In some of any of the embodiments, the kits are used in accordance with any of the provided methods. In some of any of the embodiments, the kits contain instructions for performing any of the provided methods.

In some of any embodiments, the second polynucleotide is circularized by connecting or ligating HRb1 and HRb2 using HRb′ as a splint.

In some of any embodiments, the target nucleic acid is an mRNA, and the first polynucleotide, the second polynucleotide, and the third polynucleotide are DNA molecules.

In some of any embodiments, the second polynucleotide comprises one or more barcode sequences BC1, BC2, . . . , and BCn, wherein n is an integer of 1 or greater. In some of any embodiments, the first polynucleotide comprises one or more barcode sequences BCα1, BCα2, . . . , and BCαp, wherein p is an integer of 1 or greater. In some of any embodiments, the third polynucleotide comprises one or more barcode sequences BCa1, BCa2, . . . , and BCaq, wherein q is an integer of 1 or greater.

In some of any embodiments, the kit also includes: one or more probes P1, P2, . . . , and Prn, capable of hybridizing to any one or more of BC1, BC2, . . . , and BCn, wherein m is an integer of 1 or greater and in and n are independent of each other; one or more probes Pα1, Pα2, . . . , and Pαr, capable of hybridizing to any one or more of BCα1, BCα2, . . . , and BCαp, wherein p is an integer of 1 or greater and p and r are independent of each other; and/or one or more probes Pa1, Pa2, . . . , and Pas, capable of hybridizing to any one or more of BCa1, BCa2, . . . , and BCaq, wherein s is an integer of 1 or greater and q and s are independent of each other. In some of any embodiments, in is equal to n, and P1, P2, . . . , and Pn are capable of hybridizing to BC1, BC2, . . . , and BCn, respectively; p is equal to r, and Pα1, Pα2, . . . , and Pαr are capable of hybridizing to BCα1, BCα2, . . . , and BCαp, respectively; and/or q is equal to s, and Pa1, Pa2, . . . , and Pas, are capable of hybridizing to BCa1, BCa2, . . . , and Bcaq, respectively. In some of any embodiments, one or more of P1, P2, . . . , and Pin comprise secondary barcode sequences SBC1, SBC2, . . . , and SBCi, wherein i is an integer of 1 or greater. In some of any embodiments, i is 2 or greater and SBC1, SBC2, . . . , and SBCi are different secondary barcode sequences. In some of any embodiments, the secondary barcode sequences of at least two of P1, P2, . . . , and Pm are different. In some of any embodiments, m is 1 and i is 4, and P1 comprises four different secondary barcode sequences SBC1, SBC2, SBC3, and SBC4. In some of any embodiments, in is 2 and i is 4, P1 comprises four different secondary barcode sequences SBC11, SBC12, SBC13, and SBC14, P2 comprises four different secondary barcode sequences SBC21, SBC22, SBC23, and SBC24, and wherein SBC11, SBC12, SBC13, SBC14, SBC21, SBC22, SBC23, and SBC24 are different from each other.

In some of any embodiments, the kits also include a detectably labeled detection oligonucleotide capable of hybridizing to one or more of SBC1, SBC2, . . . , and SBCi. In some of any embodiments, the kit includes a plurality of detectably labeled detection oligonucleotides capable of hybridizing to SBC1, SBC2, . . . , and SBCi, respectively. In some of any embodiments, the detectable label is a fluorescent label, and the plurality of detection oligonucleotides are labeled with the same fluorescent label, or at least two of the detection oligonucleotides are labeled with different fluorescent labels.

In some of any embodiments, the first polynucleotide, the second polynucleotide, and/or the third polynucleotide comprise an anchoring site and/or a hybridization site. In some of any embodiments, the anchoring site comprises a functional group, optionally in a modified nucleotide in the first polynucleotide, the second polynucleotide, and/or the third polynucleotide, that is capable of reacting with a matrix, such as a hydrogel.

In some of any embodiments, the kits also include the matrix or material for forming the matrix.

In some of any embodiments, the kits also include a ligase capable of ligating HRb1 and HRb2 using HRb′ as a splint, thereby circularizing the second polynucleotide.

In some of any embodiments, the kits also include a polymerase capable of using the first polynucleotide as a primer and the circularized second polynucleotide as a template to form a rolling circle amplification product.

Also provided herein are compositions that comprise a first polynucleotide, a second polynucleotide, a third polynucleotide, and a target nucleic acid. In some aspects, provided herein are compositions that comprise a first polynucleotide, a second polynucleotide, a third polynucleotide, and a target nucleic acid, wherein: the first polynucleotide comprises, in the 5′ to 3′ direction, hybridization regions HR1 and HRa′, the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3, and the first polynucleotide, the second polynucleotide, and the third polynucleotide forms a hybridization complex with the target nucleic acid which comprises, in the 3′ to 5′ direction, hybridization regions HR1′, HR2′, and HR3′, wherein in the hybridization complex, HR1, HR2, and HR3 hybridize to HR1′, HR2′, and HR3′, respectively, HRa′ hybridizes to HRa; and HRb1 and HRb2 form a split hybridization region that hybridizes to HRb′.

In some of any embodiments, the composition also includes: one or more probes P1, P2, . . . , and Pm, hybridized to any one or more of BC1, BC2, . . . , and BCn, wherein in is an integer of 1 or greater and in and n are independent of each other; one or more probes Pα1, Pα2, . . . , and Pαr, capable of hybridizing to any one or more of BCα1, BCα2, . . . , and BCαp, wherein p is an integer of 1 or greater and p and r are independent of each other; and/or one or more probes Pa1, Pa2, . . . , and Pas, capable of hybridizing to any one or more of BCa1, BCa2, . . . , and BCaq, wherein s is an integer of 1 or greater and q and s are independent of each other.

In some of any embodiments, the composition also includes: a detectably labeled detection oligonucleotide hybridized to one or more of SBC1, SBC2, . . . , and SBCi. In some of any embodiments, the composition includes: a plurality of detectably labeled detection oligonucleotides hybridized to SBC1, SBC2, . . . , and SBCi, respectively.

In some of any embodiments, the second polynucleotide is circularized.

In some of any embodiments, the composition includes: a rolling circle amplification product formed using the first polynucleotide as a primer and the circularized second polynucleotide as a template. In some of any embodiments, the rolling circle amplification product forms a DNA nanoball.

In some of any embodiments, the composition includes: at least two rolling circle amplification products. In some of any embodiments, the at least two rolling circle amplification products are directly cross-linked, cross-linked via a symmetric bi-functional cross-linker, or cross-linked via an asymmetric bi-functional cross-linker. In some of any embodiments, at least two of the hybridization complexes are formed on the same target nucleic acid molecule. In some of any embodiments, at least two of the hybridization complexes are formed on different target nucleic acid molecules.

In some of any embodiments, the methods involve amplifying the one or more barcode sequences in situ. In some of any embodiments, the amplification in situ comprises a hybridization chain reaction (HCR) directly or indirectly on the one or more barcode sequences, linear oligonucleotide hybridization chain reaction (LO-HCR) directly or indirectly on the one or more barcode sequences, primer exchange reaction (PER) directly or indirectly on the one or more barcode sequences, assembly of branched structures directly or indirectly on the one or more barcode sequences, hybridization of a plurality of detectable probes directly or indirectly on the one or more barcode sequences, or any combination thereof.

### BRIEF DESCRIPTION OF THE DRAWINGS

FIG. 1 depicts a schematic of an exemplary polynucleotide probe set in accordance with the provided embodiments, including the hybridization regions (HRs). The probe set comprises three polynucleotides: a first polynucleotide (also called “left probe”), a second polynucleotide (also called “center probe” or “padlock probe”) and a third polynucleotide (also called “right probe”). The target nucleic acid contains target sites HR1′, HR2′, and HR3′, which can hybridize to target hybridization regions HR1 of the first polynucleotide, HR2 of the second polynucleotide, and HR3 of the third polynucleotide, respectively. HRa′ of the first polynucleotide can hybridize to HRa of the second polynucleotide. HRb1 at the 5′ end and HRb2 at the 3′ end of the second polynucleotide together can hybridize to HRb′ of the third polynucleotide with HRb′ acting as a splint to bring the 5′ and 3′ ends of the second polynucleotide into proximity with one another for ligation.

FIG. 2 depicts a schematic of an exemplary polynucleotide probe set in accordance with the provided embodiments, depicting potential additional components of the three polynucleotides described in, e.g., FIG. 1. Certain components such as modifications and barcodes depicted in hashed marks and indicated in parentheses are features that can be optional. The 3′ end of the first polynucleotide is used as a primer for rolling circle amplification (RCA) of the second polynucleotide (“RCA primer”). The first polynucleotide can also contain one or more barcode(s) and/or one or more modification(s), for example at the 5′ end, that can serve as an anchoring site. The second polynucleotide can be connected (e.g., circularized), for example, by ligation, with HRb1 at the 5′ end and HRb2 at the 3′ end hybridizing to HRb′ of the third polynucleotide, with HRb′ acting as a splint, and amplified by RCA using, e.g., the first polynucleotide as a primer (with the arrow indicating the direction of RCA). The second polynucleotide can also contain one or more barcode(s), that can be amplified and used for detection and/or sequencing. The amplification product generated from the second polynucleotide also can contain one or more modification(s), for example by incorporation of modified nucleotides during RCA, that can serve as an anchoring site. It should be appreciated that the modified nucleotides may be in the RCA product and the second polynucleotide itself does not need to contain the one or more modification(s). The third polynucleotide provides HRb′ that functions as a splint for ligation and circularization of the second polynucleotide, and can also contain one or more barcode(s) and/or one or more modification(s), for example at the 3′ end, that can serve as an anchoring site.

FIG. 3 shows a schematic depicting an example of potential barcodes that can be included in each of the polynucleotides in the probe set, and their use to distinguish particular target sites and/or particular target nucleic acids.

FIG. 4 shows a schematic depicting an example of potential anchoring site(s) and/or hybridization site(s) that can be used to anchor the polynucleotide probe set, amplification product and/or target nucleic acid to a scaffold or a matrix. Diamonds represent modifications or functional groups that provide reactive groups and/or sites for anchoring or attaching to the matrix. Lines connecting the modifications or functional groups in the scaffold/matrix represent oligonucleotides, polymers or chemical groups that act as the network for the scaffold or matrix. In some embodiments, the matrix comprises a hydrogel. In some embodiments, the matrix comprises one or more nucleic acids and the hybridization site(s) on the polynucleotide probes may hybridize to the nucleic acid matrix.

FIG. 5 shows a schematic depicting an example of anchoring or cross-linking of the polynucleotide probe set, amplification product (e.g., nanoball) and/or target nucleic acid to another nucleic acid molecule (another polynucleotide probe set, amplification product (e.g., nanoball) and/or target nucleic acid). Diamonds represent modifications or functional groups that provide reactive groups and/or sites for anchoring, attaching or cross-linking to another nucleic acid molecule. The cross-linking can be direct, or indirect, via a linker, such as a symmetric bifunctional cross linker or an asymmetric bifunctional cross linker.

### DETAILED DESCRIPTION

Provided herein are methods involving the use of a set of polynucleotides, such as one or more polynucleotide(s), for analyzing one or more target nucleic acid(s), such as a target nucleic acid (for example, a messenger RNA) present in a cell or a biological sample, such as a tissue sample. Also provided are polynucleotides, sets of polynucleotides, compositions, kits, systems and devices for use in accordance with the provided methods. In some aspects, the provided methods can be applied to detect, image, quantitate or determine the sequence of one or more target nucleic acid(s). In some aspects, the provided embodiments can be employed for in situ detection and/or sequencing of a target nucleic acid in a cell in a biological sample, such as a tissue sample, or a sample derived from a biological sample, such as a tissue section on a solid support, such as on a slide.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. OVERVIEW

In some aspects, the provided methods, kits and compositions that involve the use of a set of polynucleotide probes, such as a set of at least three polynucleotides. In some aspects, the probe set comprises three polynucleotides: a first polynucleotide, a second polynucleotide, and a third polynucleotide, for analyzing a target nucleic acid. In some aspects, the three-polynucleotide probe set contains hybridization regions (HRs) that hybridizes to target sites in the target nucleic acids (e.g., mRNA in a cell), and also HRs that hybridize to one or more other polynucleotides in the polynucleotide probe set. In some aspects, one or more polynucleotide(s) of the probe set are amplified. In some aspects, one or more polynucleotide(s) in the probe set are modified for attaching, anchoring or linking the polynucleotide probe set, the target nucleic acid, hybridization complexes and/or amplification products to particular locations in the cell and/or the sample; such as to a matrix, other nucleic acids present in the sample, and/or to other cellular structures in the sample. In some aspects, provided herein are methods for analyzing one or more target nucleic acids of interest in a biological sample, e.g., a tissue sample. In some embodiments, the methods, kits and compositions involve analyzing the presence/absence, distribution, location, amount, level, or expression of target nucleic acids in a tissue sample in situ. In some aspects, the provided methods can be applied for various applications, including for in situ analysis, including in situ detection and/or sequencing of target nucleic acids and multiplexed nucleic acid analysis.

In some aspects, existing methods for assessing nucleic acids, particularly for in situ analysis and/or for multiplexed analysis, can have certain limitations. For example, certain padlock probes, which have been used as an alternative to PCR- or hybridization-based methods for in situ analysis of nucleic acids, in some cases have low specificity and low efficiency of ligation, particularly when the ligation template is an RNA template. Padlock probes that use the target mRNA as a template for ligation (e.g., DNA-RNA or RNA-RNA templated ligation) can also result in the formation of secondary structures that hinder the amplification or detection steps, or limit the target site for detection to particular positions in the mRNA. Other methods may also have limitations such as low sensitivity or specificity, limited plexity, biases, high costs and/or are time-consuming, labor-intensive, and/or error-prone. Also, in some cases, when polynucleotide probes are employed, the length of the polynucleotide can be limited by accuracy, fidelity, length and costs of generating the polynucleotide probes. In some aspects, as various different functionalities are included in certain polynucleotide probes, the practical limits on accuracy, fidelity, length and costs of generating the polynucleotides, e.g., by synthesis, can limit the function of the polynucleotides and downstream analysis processes.

Provided are methods, polynucleotides, set of polynucleotides, compositions, kits, devices and systems, that overcome some or all of these limitations. For example, the described embodiments can provide an improved, faster, more efficient and more accurate way to perform analysis of nucleic acid molecules, such as for in situ analyses, such as in situ sequencing. In some aspects, the provided embodiments provide for a faster processing time, higher multiplexity, higher efficiency, higher sensitivity, lower error rate, reduced costs, higher accuracy, higher spatial resolution and higher versatility for downstream applications, compared to existing methods for in situ nucleic acid analysis.

The provided embodiments permits highly sensitive and specific detection and sequencing of nucleic acid sequences in highly complex samples, for example, in intact biological tissue containing numerous different mRNA sequences, with single-cell and single molecule sensitivity, preserved tissue morphology, and/or high signal-to-noise ratio with low error rates.

In some aspects, the three-polynucleotide probe set provides advantages of high specificity, as all three polynucleotides must be in proximity and hybridize to the target polynucleotide, for amplification and detection to occur.

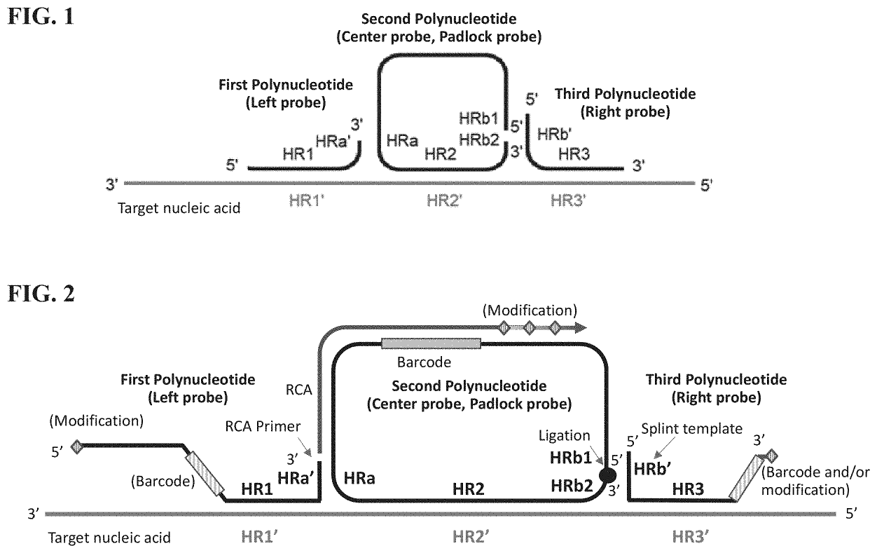

In some embodiments, the three-polynucleotide probe set provides the advantage of uncoupling when ligation and amplification steps occur (e.g., such that they are two separate steps). In addition, by uncoupling the ligation, for circularizing a probe polynucleotide for amplification, and the priming of the amplification into two different polynucleotides, the methods further increase specificity. For instance, all three of the left, center, and right probes shown in FIG. 1 must hybridize to the target nucleic acid, and the left probe to the center probe and center probe to the right probe, in order for productive RCA to occur. On the one hand, if only the left probe (e.g., a probe comprising an RCA primer) and the center probe (e.g., a padlock probe) are hybridized, the left probe can be extended using the center probe as template but no RCA product would be generated, because the center probe would not be circularized in the absence of the right probe (e.g., a probe comprising a splint for padlock ligation). On the other hand, if only the center probe and the right probe are hybridized, the center probe can be circularized but no RCA product would be generated in the absence of the left probe priming the reaction. Finally, no RCA product would be generated when only the left and right probes are hybridized to the target nucleic acid.

In some embodiments, the methods disclosed herein provide additional space in the probe polynucleotides for functionalizing, addition of barcodes and/or anchoring, for further downstream assessment. For example, the amplification products can be anchored or cross-linked to a matrix, or to other amplification products or nucleic acids. In addition, the increased space for barcodes in the probe polynucleotide can increase the potential and efficiency for decoding schemes, increase multiplexing potential, reduce errors, and decrease issues of optical crowding, to permit more efficient and higher resolution analysis. In some aspects, such increase in efficiency can also reduce the image acquisition time, which is often a bottle neck for in situ analysis methods involving microscopy.

Using three or more separate polynucleotides as probes with separate functions, also can improve the accuracy and specificity and reduce cost, by reducing the length of polynucleotides that need to be synthesized. In some cases, the fidelity and accuracy can decrease, and the costs can increase, when longer polynucleotides need to be synthesized. In some aspects, compared to certain methods utilizing two polynucleotide probe sets such as a SNAIL (Splint Nucleotide Assisted Intramolecular Ligation) probe set, each of the polynucleotide probes of the present disclosure can be shorter. In a SNAIL probe set, each primer or padlock probe typically has 19-25 nucleotides to hybridize with a target nucleic acid sequence (a total hybridization region of 38-50 nucleotides in length for both the primer and the padlock). In some embodiments, the polynucleotide probes disclosed herein can have shorter target hybridization regions. For example, independent of each other, the polynucleotide probes disclosed herein can each have a target hybridization region that is between about 5 and about 18 nucleotides, for example, about 7, 8, 9, 10, 11, 12, 13, 14, 15, or 16 nucleotides in length. Accordingly, the length of each probe length that needs to be synthesized can be reduced, increasing accuracy and reducing errors and costs. In some embodiments, for a particular individual probe, a shorter target hybridization region may increase, decrease, or not substantially alter hybridization specificity of the probe to target nucleic acid sequences in a sample. However, in these embodiments, the overall detection specificity can still be increased compared to a SNAIL probe set, since the methods disclosed herein require hybridization of all three probes (e.g., a probe comprising an RCA primer, a padlock probe, a probe comprising a splint for padlock ligation) to a target nucleic acid, as well as hybridization between two adjacent probes, in order to produce an RCA product that is subsequently detected.

Further, in view of the location of the hybridization region in the amplified probe and the direction of synthesis, the probe set can minimize the binding or amplification of incomplete probes that can reduce the signal. When a padlock probe of the SNAIL probe set is synthesized in the 5′ to 3′ direction, the target hybridization region is at the 5′ end and is generally accurate and can hybridize to the target. However, the 3′ end of the padlock probe is more likely to contain errors and is often truncated, reducing the ability of the padlock probe to be properly ligated. These unproductive probes can occupy the target sites on the target nucleic acid and may displace the productive probes, leading to lower yield and efficiency of subsequent amplification and detection. By virtue of the presence of having the target hybridization region on the second polynucleotide (padlock probe) towards the 3′ end, the provided methods can reduce or prevent incomplete or truncated second polynucleotides (see, e.g., FIG. 1), for example, that are missing part of the 3′ end, from hybridizing and occupying the target site, which may normally reduce the signal. Accordingly, the provided methods, polynucleotides, set of polynucleotides, compositions, kits, devices and systems can improve sensitivity and signal strength without being affected by incomplete or inaccurate synthesis of polynucleotides.

In some aspects, the provided methods also permit higher resolution for image-based quantification of nucleotide expression at cellular and even subcellular resolution (e.g., in tissue sample, such as a tissue section). For example, in some cases, the anchoring steps permit high-resolution volumetric imaging and analysis compatible with other downstream applications, such as analysis of proteins, nucleic acids, and other targets and further reactions, such as enzymatic reactions. In some aspects, embedding the biological sample with particular polymer matrices can also provide optical transparency, reduced background, elevated diffusion rate, and greater mechanical stability. In some aspects, the provided embodiments also permit the assessment of biological samples without the need for additional embedding steps, by direct anchoring and/or linking of the amplification products. In some aspects, the methods can be used to preserve the spatial information, increase mechanical stability of analytes, increase optical transparency and increase permeability, for example, for reagents and components for detection. Thus, the provided methods, polynucleotides, set of polynucleotides, compositions, kits, devices and systems provide numerous advantages compared to existing analysis methods.

### II. METHODS FOR ANALYZING A TARGET NUCLEIC ACID

Provided herein are methods for analyzing one or more target nucleic acid(s), such as a messenger RNA (mRNA). In some aspects, the provided methods can be employed for assessing one or more target nucleic acid(s), such as a plurality of different mRNAs, in a biological sample, such as a cell or a tissue sample (such as a tissue section). In some aspects, the provided methods are employed for in situ analysis of target nucleic acids, for example for in situ sequencing or multiplexed analysis in intact tissues or a sample that preserves the cellular or tissue structure.

In some aspects, the provided methods involve a step of contacting, or hybridizing, one or more of the probe polynucleotides, such as the first polynucleotide, the second polynucleotide, and/or the third polynucleotide, to a cell or a sample, such as a biological sample, containing analytes, such as the target nucleic acid, to form a hybridization complex (see, e.g., FIGS. 1-3). In some aspects, the provided methods involve a step of connecting or circularizing, or for example by ligating the ends of, one of the polynucleotides in the probe set (e.g., the second polynucleotide), to form a circularized polynucleotide probe. In some aspects, the provided methods involve a step of amplifying one of the polynucleotides in the probe set (e.g., the circularized second polynucleotide), to generate an amplification product. In some aspects, the provided methods involve a step of detecting and/or determining the sequence of all or a portion of the amplification product (for example, one or more barcode(s)) and/or one or more of the polynucleotide probe(s). In some aspects, the provided methods involve a step of anchoring or linking the polynucleotide probe set, the target nucleic acid, hybridization complexes and amplification products to particular locations in the cell and/or the sample, such as to a matrix, other nucleic acids present in the sample and/or to cellular structures. In some aspects, the provided methods involve performing one or more of the steps described herein, simultaneously and/or sequentially.

In some embodiments, the provided methods involve one or more steps of: contacting a target nucleic acid with a first polynucleotide, a second polynucleotide, and a third polynucleotide to form a hybridization complex; circularizing the second polynucleotide; forming an amplification product using the circularized second polynucleotide as a template and the first polynucleotide as a primer; and/or detecting the amplification product. In some aspects, the detection is indicative of the target nucleic acid or a sequence thereof. In some embodiments, the provided methods involve contacting a target nucleic acid with a first polynucleotide, a second polynucleotide, and a third polynucleotide to form a hybridization complex; circularizing the second polynucleotide; forming an amplification product using the circularized second polynucleotide as a template and the first polynucleotide as a primer; and detecting the amplification product, wherein the detection is indicative of the target nucleic acid or a sequence thereof, performed in such order. Particulars of the steps of the methods can be carried out as described herein, for example in Section IV; and/or using any suitable processes and methods for carrying out the particular steps.

In some of the provided embodiments, a three-polynucleotide probe set is used. In some aspects, the three-polynucleotide probe set comprises: a first polynucleotide, a second polynucleotide, and a third polynucleotide. Schematics depicting the exemplary three-polynucleotide probe set and the hybridization regions (HRs) and other components of the polynucleotides in the exemplary probe set is shown in FIGS. 1 and 2. In some embodiments, the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; and the third polynucleotide comprises, in the 5′ to 3′ direction, hybridization regions HRb′ and HR3. In some embodiments, HRa′ hybridizes to HRa. In some embodiments, HRb′ forms a split hybridization region that hybridizes to HRb1 and HRb2. In some embodiments, the second polynucleotide is circularized by connecting (e.g., ligating) HRb1 and HRb2 using HRb′ as a splint. In some aspects, the target nucleic acid comprises, in the 3′ to 5′ direction, hybridization regions HR1′, HR2′, and HR3′. In some aspects, HR1, HR2, and HR3 hybridize to HR1′, HR2′, and HR3′. Accordingly, by virtue of the contacting and hybridization step, the target nucleic acid and the first polynucleotide, the second polynucleotide and the third polynucleotide of the probe set generates a hybridization complex, with the first polynucleotide, the second polynucleotide and the third polynucleotide binding to the target polynucleotide in a linear fashion. Particulars of the features of the polynucleotides in the probe set is described herein, for example in Section III.

In some aspects, provided are methods for analyzing a target nucleic acid that involves contacting a target nucleic acid with a first polynucleotide, a second polynucleotide, and a third polynucleotide to form a hybridization complex, wherein: the first polynucleotide comprises, in the 5′ to 3′ direction, hybridization regions HR1 and HRa′; the second polynucleotide comprises, in the 5′ to 3′ direction, hybridization regions HRb1, HRa, HR2, and HRb2; the third polynucleotide comprises, in the 5′ to 3′ direction, hybridization regions HRb′ and HR3; the target nucleic acid comprises, in the 3′ to 5′ direction, hybridization regions HR1′, HR2′, and HR3′; HR1, HR2, and HR3 hybridize to HR1′, HR2′, and HR3′, respectively; HRa′ hybridizes to HRa; and HRb1 and HRb2 form a split hybridization region that hybridizes to HRb′; circularizing the second polynucleotide by connecting HRb1 and HRb2 using HRb′ as a splint; forming an amplification product using the circularized second polynucleotide as a template and the first polynucleotide as a primer; and analyzing a sequence in an amplification product, wherein the sequence is indicative of a target nucleic acid or a sequence thereof.

In some embodiments, the methods also involve attaching, anchoring or linking the polynucleotide probe set, the target nucleic acid, hybridization complexes and amplification products to particular locations in the cell and/or the sample, such as to a matrix, other nucleic acids present in the sample and/or to cellular structures. In some aspects, parts of the polynucleotides and/or amplified products are modified to facilitate the attaching, anchoring or linking. In some aspects, the methods also involve additional processing or manipulation of the samples, such as by embedding the sample in a matrix. Particulars of the features of the modified polynucleotides and steps for processing or manipulating the samples, including for attaching or anchoring, is described herein, for example in Sections III and V.

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify of diverse anatomically- and molecularly-resolved cell types within particular tissues. In some embodiments, the methods can be applied for a three-dimensional (3D) tissue RNA sequencing. In some aspects, the embodiments can be applied in assessing the cellular and/or tissue architecture of a cell, a tissue or an organ from an organism.

In some aspects, the embodiments can be applied in investigative and/or diagnostic applications, for example, for characterization or assessment of particular cell or a tissue from a subject. Applications of the provided method can include biomedical research and clinical diagnostics. For example, in biomedical research, applications include, but are not limited to, spatially resolved gene expression analysis for biological investigation or drug screening. In clinical diagnostics, applications include, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry analysis.

### III. POLYNUCLEOTIDE PROBES

Provided herein are polynucleotides and sets of polynucleotides as described herein. Also provided herein are polynucleotides and sets of polynucleotides for use in accordance with the methods described herein. In some aspects, the set of polynucleotides comprises a plurality of polynucleotides, such as at least three polynucleotides. In some aspects, the set of polynucleotides comprises three different polynucleotides. In some aspects, the three polynucleotides together are used as a probe to analyze an analyte, such as a target nucleic acid (for example, messenger RNA in a cell or a biological sample). In some aspects, provided are sets of polynucleotides that each comprise three polynucleotide probes, for example, a first polynucleotide, a second polynucleotide and a third polynucleotide.

FIGS. 1 and 2 depict schematics of an exemplary polynucleotide probe set. In some aspects, the probe sets include three polynucleotides: a first polynucleotide (also called “left probe”), a second polynucleotide (also called “center probe” or “padlock probe”) and a third polynucleotide (also called “right probe”).

Each of the polynucleotides in the probe set includes two or more hybridization regions (HRs). Exemplary schematics of the HR regions comprised in the polynucleotide probe set are depicted in FIGS. 1 and 2. The HRs refer to regions that are complementary or sufficiently complementary to a different nucleic acid sequence (for example, to the target nucleic acid, such as an mRNA, or to another HR present in one or a different polynucleotide of the probe set) to form complexes via, e.g., Watson-Crick base pairing. In accordance with the provided embodiments, the polynucleotides are designed to permit hybridization of the HRs and each of the polynucleotides or regions in the polynucleotides in the probe set to provide the structural elements for amplification as described below.

In some aspects, the terms “polynucleotide probe set” and “probe set” and their plural forms are synonymous and are used interchangeably throughout this specification. In some aspects, a single polynucleotide probe set can be used, for example, when a single target nucleic acid or a single variant in a RNA are to be detected. A single polynucleotide probe set can be used to detect one target nucleic acid among a plurality of target nucleic acids that may be present in a sample. In some aspects, a plurality of polynucleotide probe sets, such as those containing different target site sequences. In some aspects, the different polynucleotide probe sets can target a variety of different target site sequences on the same target nucleic acid, and/or target a variety of different target nucleic acids or variants. For multiplexed analysis, two or more different target nucleic acids are to be detected in a sample. In some embodiments, the sample of cells is contacted with a plurality of polynucleotide probe sets for each target a different target nucleic acid.

In some embodiments, the methods described herein support multiplexed spatial analysis. In some embodiments, spatial analysis of a biological analyte can be performed individually for each analyte of interest. In some embodiments, multiple biological analytes can be detected and spatially analyzed simultaneously within the same biological sample. In some embodiments, multiplexing (e.g., simultaneously detecting multiple markers) allows for examination of spatial arrangement of analytes of interest (e.g., proteins, nucleic acids, such as DNA, RNA) as well as analyte interaction and co-localization thereby facilitating simultaneous analysis of multiple tissue markers. In some embodiments, multiple biological analytes can be detected and spatially analyzed at different times (e.g., detection and analysis of one category of analyte, followed by detection and analysis of another category of analyte, or detection and analysis of one analyte followed by detection and analysis of another analyte). For example, detection, examination, and analysis of one or more target nucleic acids, such as mRNA transcripts, within a biological sample can occur prior to the detection, examination, and analysis of one or more target nucleic acids, such as mRNA transcripts.

In some embodiments, the methods, polynucleotides, set of polynucleotides, compositions, kits, devices and systems described herein employ a plurality of first polynucleotides, including, but not limited to, 5 or more first polynucleotides, e.g., 8 or more, 10 or more, 12 or more, 15 or more, 18 or more, 20 or more, 25 or more, 30 or more, 35 or more, such as 50 or more, 100 or more, 500 or more, 1000 or more, 2000 or more, 3000 or more, 4000 or more, 5000 or more, 10000 or more, 20000 or more, 30000 or more, 40000 or more, or 50000 or more, that hybridize to target nucleotide sequences. In some embodiments, the methods employ a plurality of first polynucleotides, including, but not limited to, 15 or more first polynucleotides, e.g., 20 or more, 30 or more, 40 or more, 50 or more, 60 or more, 70 or more, 80 or more, 90 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 40000 or more, or 50000 or more, first polynucleotides that hybridize to 15 or more, e.g., 20 or more, 30 or more, 40 or more, 50 or more, 60 or more, 70 or more, 80 or more, 90 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 40000 or more, or 50000 or more, different target nucleotide sequences.

In some embodiments, the methods, polynucleotides, set of polynucleotides, compositions, kits, devices and systems described herein include a plurality of second polynucleotides, including, but not limited to, 5 or more second polynucleotides, e.g., 8 or more, 10 or more, 12 or more, 15 or more, 18 or more, 20 or more, 25 or more, 30 or more, 35 or more, such as 50 or more, 100 or more, 500 or more, 1000 or more, 2000 or more, 3000 or more, 4000 or more, 5000 or more, 10000 or more, 20000 or more, 30000 or more, 40000 or more, or 50000 or more, that hybridize to target nucleotide sequences. In some embodiments, a method of the present disclosure includes a plurality of second polynucleotides including, but not limited to, 15 or more second polynucleotides, e.g., 20 or more, 30 or more, 40 or more, 50 or more, 60 or more, 70 or more, 80 or more, 90 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 40000 or more, or 50000 or more, second polynucleotides that hybridize to 15 or more, e.g., 20 or more, 30 or more, 40 or more, 50 or more, 60 or more, 70 or more, 80 or more, 90 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 40000 or more, or 50000 or more, different target nucleotide sequences.

In some embodiments, the methods, polynucleotides, set of polynucleotides, compositions, kits, devices and systems described herein include a plurality of third polynucleotides, including, but not limited to, 5 or more third polynucleotides, e.g., 8 or more, 10 or more, 12 or more, 15 or more, 18 or more, 20 or more, 25 or more, 30 or more, 35 or more, such as 50 or more, 100 or more, 500 or more, 1000 or more, 2000 or more, 3000 or more, 4000 or more, 5000 or more, 10000 or more, 20000 or more, 30000 or more, 40000 or more, or 50000 or more, that hybridize to target nucleotide sequences. In some embodiments, a method of the present disclosure includes a plurality of third polynucleotides including, but not limited to, 15 or more third polynucleotides, e.g., 20 or more, 30 or more, 40 or more, 50 or more, 60 or more, 70 or more, 80 or more, 90 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 40000 or more, or 50000 or more, third polynucleotides that hybridize to 15 or more, e.g., 20 or more, 30 or more, 40 or more, 50 or more, 60 or more, 70 or more, 80 or more, 90 or more, 100 or more, 200 or more, 300 or more, 400 or more, 500 or more, 1000 or more, 2000 or more, 3000 or more, 4000 or more, 5000 or more, 10000 or more, 20000 or more, 30000 or more, 40000 or more, or 50000 or more, different target nucleotide sequences.

In some embodiments, a plurality of set of polynucleotides can be used in a reaction, where one or more sets specifically bind to each target nucleic acid. For example, two polynucleotide sets can be used for one target nucleic acid, for example to improve sensitivity and reduce variability. It is also of interest to detect a plurality of different target nucleic acids in a cell, e.g., detecting more than 2, more than 3, more than 4, more than 5, more than 6, more than 7, more than 8, more than 9, more than 10, more than 12, more than 15, more than 18, more than 20, more than 25, more than 30, more than 40, more than 50, more than 60, more than 70, more than 80, more than 90, more than 100, more than 200, more than 300, more than 400, more than 500, more than 1000, more than 2000, more than 3000, more than 4000, more than 5000, more than 10000, more than 20000, more than 30000, more than 40000, more than or 50000 or more, distinct target nucleic acids.

As used herein, the term “nucleic acid” includes the term “oligonucleotide” or “polynucleotide” which includes a string containing a plurality of nucleotides. The terms “nucleic acid” or “nucleic acids” are intended to include naturally occurring nucleic acids and synthetic nucleic acids. The terms “nucleic acid” or “nucleic acids” are intended to include single stranded nucleic acids and double stranded nucleic acids. The terms “nucleic acid” or “nucleic acids” are intended to include DNA and RNA, whether single stranded or double stranded. Nucleotides of the present embodiments will typically be the naturally-occurring nucleotides such as nucleotides derived from adenosine, guanosine, uridine, cytidine and thymidine. When oligonucleotides are referred to as “double-stranded,” it is understood by a skilled person that a pair of oligonucleotides exists in a hydrogen-bonded, helical array typically associated with, for example, DNA. In addition to the 100% complementary form of double-stranded oligonucleotides, the term “double-stranded” as used herein is also meant to include those form which include such structural features as bulges and loops (see Stryer, Biochemistry, Third Ed. (1988), incorporated herein by reference in its entirety for all purposes). As used herein, the term “polynucleotide” refers to a strand of nucleic acids that can be a variety of different sizes.

Polynucleotides or oligonucleotides may be isolated from natural sources or purchased from commercial sources. Polynucleotide sequences or oligonucleotide sequences may be prepared by any suitable method, e.g., the phosphoramidite method described by Beaucage and Carruthers ((1981) Tetrahedron Lett. 22: 1859) or the triester method according to Matteucci et al. (1981) J. Am. Chem. Soc. 103:3185), both incorporated herein by reference in their entirety for all purposes, or by other chemical methods using either a commercial automated oligonucleotide synthesizer or high-throughput, high-density array provided embodiments and known (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

In some embodiments, polynucleotides, such as one or more of the polynucleotide probe(s), or oligonucleotides, may be prepared using a variety of known microarray technologies. Pre-synthesized polynucleotide sequences or oligonucleotides may be attached to a support or synthesized in situ using light-directed methods, flow channel and spotting methods, inkjet methods, pin-based methods and bead-based methods set forth in the following references: McGall et al. (1996) Proc. Natl. Acad. Sci. USA. 93:13555; Synthetic DNA Arrays In Genetic Engineering, Vol. 20:111, Plenum Press (1998); Duggan et al. (1999) Nat. Genet. S21:10; Microarrays: Making Them and Using Them in Microarray Bioinformatics, Cambridge University Press, 2003; US 2003/0068633; US 2002/0081582; US 2003/0087298; US 2003/0099952; US 2004/0126757; U.S. Pat. Nos. 6,833,450, 6,830,890, 6,824,866, 6,800,439, 6,375,903; 5,700,637; 7,037,659; 7,157,229; 7,422,851; 7,498,176; 7,846,660; 7,888,106; 8,026,094; and 8,129,196; incorporated herein by reference in their entirety for all purposes.

Polynucleotides may be obtained from libraries. Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, incorporated herein by reference in their entirety for all purposes.

A. First Polynucleotide

In some aspects, the polynucleotide probe set, such as a three-polynucleotide probe set, includes a first polynucleotide. In some aspects, the first polynucleotide can also be referred to as a “left probe.” FIGS. 1 and 2 depict schematics of an exemplary probe set, including the first polynucleotide (left probe).

In some embodiments, the first polynucleotide comprises: a target hybridization region (for example, referred to as HR1 in the exemplary schematic depicted in FIGS. 1 and 2) that hybridizes to a complementary (or near-complementary) target site (for example, referred to as HR1′ in the exemplary schematic depicted in FIGS. 1 and 2) in the target nucleic acid; and a different hybridization region (for example, referred to as HRa′ in the exemplary schematic depicted in FIGS. 1 and 2) that hybridizes to a hybridization region in the second polynucleotide (for example, referred to as HRa in the exemplary schematic depicted in FIGS. 1 and 2). In some embodiments, the first polynucleotide contains, in the 5′ to 3′ direction, hybridization regions HR1 and HRa′. In some instances, the HRa′ and HRa interaction is not used as a splint for ligation of the second polynucleotide. Thus, in some embodiments, the HRa′ and HRa interaction is utilized for specificity of the probe set for the target nucleic acid, but not for ligation of the second polynucleotide (e.g., the second polynucleotide does not comprise a nick or gap in HRa).

In some embodiments, the first polynucleotide comprises a target hybridization region (e.g., HR1) that hybridizes to a complementary (or near-complementary) target site (e.g., HR1′) in the target nucleic acid. This region provides a part of the specificity for recognizing and target nucleic acid sequence (e.g., mRNA). In some aspects, the hybridization between HR1/HR1′, together with the hybridization between HR2/HR2′ and HR3/HR3′, provides the specificity for recognition and detection of particular target nucleic acid (e.g., mRNA).

In some embodiments, the length of each of HR1 of the first polynucleotide and the complementary HR1′ on the target nucleic acid, is about 4 to about 40 nucleotides, such as about 4 to about 16 nucleotides or about 8 to about 12 nucleotides. In some aspects, the length of each of HR1 and HR1′ is about 8 nucleotides. In some aspects, the length of each of HR1 and HR1′ is about 9 nucleotides. In some aspects, the length of each of HR1 and HR1′ is about 10 nucleotides. In some aspects, the length of each of HR1 and HR1′ is about 11 nucleotides. In some aspects, the length of each of HR1 and HR1′ is about 12 nucleotides. In some aspects, the length of each of HR1 and HR1′ is about 13 nucleotides. In some aspects, the length of each of HR1 and HR1′ is about 14 nucleotides. In some aspects, the length of each of HR1 and HR1′ is about 15 nucleotides. In some aspects, the length of each of HR1 and HR1′ is about 16 nucleotides. In some aspects, the length of each of HR1 and HR1′ is about 17 nucleotides. In some aspects, the length of each of HR1 and HR1′ is about 18 nucleotides. In some aspects, the length of each of HR1 and HR1′ is about 19 nucleotides. In some aspects, the length of each of HR1 and HR1′ is about 20 nucleotides. In some aspects, the length of each of HR1 and HR1′ is fewer than 8 nucleotides. In some aspects, the length of each of HR1 and HR1′ is fewer than 9 nucleotides. In some aspects, the length of each of HR1 and HR1′ is fewer than 10 nucleotides. In some aspects, the length of each of HR1 and HR1′ is fewer than 11 nucleotides. In some aspects, the length of each of HR1 and HR1′ is fewer than 12 nucleotides. In some aspects, the length of each of HR1 and HR1′ is fewer than 13 nucleotides. In some aspects, the length of each of HR1 and HR1′ is fewer than 14 nucleotides. In some aspects, the length of each of HR1 and HR1′ is fewer than 15 nucleotides. In some aspects, the length of each of HR1 and HR1′ is fewer than 16 nucleotides. In some aspects, the length of each of HR1 and HR1′ is fewer than 17 nucleotides. In some aspects, the length of each of HR1 and HR1′ is fewer than 18 nucleotides. In some aspects, the length of each of HR1 and HR1′ is fewer than 19 nucleotides. In some aspects, the length of each of HR1 and HR1′ is fewer than 20 nucleotides.

In some embodiments, the first polynucleotide comprises a hybridization region that hybridizes to a complementary (or near-complementary) region in the second polynucleotide of the probe set. In some aspects, HRa′ of the first polynucleotide can hybridize to HRa of the second polynucleotide (with reference to HR regions as depicted in FIGS. 1 and 2). In some embodiments, the first polynucleotide is single-stranded. In other embodiments, the first polynucleotide is partially double-stranded, i.e., comprises a single-stranded region. In some aspects, the first polynucleotide contains a 3′ hydroxyl (—OH) group. In some aspects, the 3′ end sequences of the first polynucleotide (e.g., HRa′ that hybridizes to a complementary region in the second polynucleotide) and the 3′-OH group of the first polynucleotide serves as a primer and the 3′-OH for beginning rolling circle amplification of the second polynucleotide. A “primer” is a single-stranded nucleic acid sequence having a 3′ end that can be used as a chemical substrate for a nucleic acid polymerase in a nucleic acid extension reaction.

In some embodiments, the length of each of HRa′ of the first polynucleotide and the complementary HRa of the second polynucleotide is at least 1 nucleotide, such as at least 2 nucleotides. In some aspects, the length of each of HRa′ and HRa is in sufficient length to provide the 3′-OH required for beginning rolling circle amplification. In some aspects, the length of each of HRa′ and HRa is about 3 to about 40 nucleotides, such as about 4 to about 20 nucleotides. In some aspects, the length of each of HRa′ and HRa is about 3 nucleotides. In some aspects, the length of each of HRa′ and HRa is about 4 nucleotides. In some aspects, the length of each of HRa′ and HRa is about 5 nucleotides. In some aspects, the length of each of HRa′ and HRa is about 6 nucleotides. In some aspects, the length of each of HRa′ and HRa is about 7 nucleotides. In some aspects, the length of each of HRa′ and HRa is about 8 nucleotides. In some aspects, the length of each of HRa′ and HRa is about 9 nucleotides. In some aspects, the length of each of HRa′ and HRa is about 10 nucleotides. In some aspects, the length of each of HRa′ and HRa is about 11 nucleotides. In some aspects, the length of each of HRa′ and HRa is about 12 nucleotides. In some aspects, the length of each of HRa′ and HRa is about 13 nucleotides. In some aspects, the length of each of HRa′ and HRa is about 14 nucleotides. In some aspects, the length of each of HRa′ and HRa is about 15 nucleotides. In some aspects, the length of each of HRa′ and HRa is about 8 nucleotides. In some aspects, the length of each of HRa′ and HRa is about 16 nucleotides. In some aspects, the length of each of HRa′ and HRa is about 17 nucleotides. In some aspects, the length of each of HRa′ and HRa is about 18 nucleotides. In some aspects, the length of each of HRa′ and HRa is about 19 nucleotides. In some aspects, the length of each of HRa′ and HRa is about 20 nucleotides. In some aspects, the length of each of HRa′ and HRa is fewer than 4 nucleotides. In some aspects, the length of each of HRa′ and HRa is fewer than 5 nucleotides. In some aspects, the length of each of HRa′ and HRa is fewer than 6 nucleotides. In some aspects, the length of each of HRa′ and HRa is fewer than 7 nucleotides. In some aspects, the length of each of HRa′ and HRa is fewer than 8 nucleotides. In some aspects, the length of each of HRa′ and HRa is fewer than 9 nucleotides. In some aspects, the length of each of HRa′ and HRa is fewer than 10 nucleotides. In some aspects, the length of each of HRa′ and HRa is fewer than 11 nucleotides. In some aspects, the length of each of HRa′ and HRa is fewer than 12 nucleotides. In some aspects, the length of each of HRa′ and HRa is fewer than 13 nucleotides. In some aspects, the length of each of HRa′ and HRa is fewer than 14 nucleotides. In some aspects, the length of each of HRa′ and HRa is fewer than 15 nucleotides. In some aspects, the length of each of HRa′ and HRa is fewer than 8 nucleotides. In some aspects, the length of each of HRa′ and HRa is fewer than 16 nucleotides. In some aspects, the length of each of HRa′ and HRa is fewer than 17 nucleotides. In some aspects, the length of each of HRa′ and HRa is fewer than 18 nucleotides. In some aspects, the length of each of HRa′ and HRa is fewer than 19 nucleotides. In some aspects, the length of each of HRa′ and HRa is fewer than 20 nucleotides.

In some embodiments, the melting temperature (Tₘ) of the hybridization between HRa′ of the first polynucleotide and the complementary HRa of the second polynucleotide is lower than the Tₘ of hybridization of HR between the probe polynucleotides and target site on the target nucleic acid (e.g., HR1/HR1′, HR2/HR2′ or HR3/HR3′). In some aspects, such differences in Tₘ permits the hybridization between the probe polynucleotides and target site on the target nucleic acid while reducing or minimizing the inter-polynucleotide probe hybridization (e.g., between HRa′-HRa) during the hybridization step. In some aspects, such differences in Tₘ permits the hybridization between the probe polynucleotides and target site on the target nucleic acid to be maintained during the methods involving the binding of the primer (e.g., 3′ HRa′ sequence) and amplification of the second polynucleotide by rolling circle amplification, and/or other subsequent steps. In some aspects, the amplification and/or subsequent steps can be performed at a temperature that is lower than the Tₘ of hybridization of HRs between the probe polynucleotides and target site on the target nucleic acid, at a temperature required for the annealing of the primer and amplification. In some aspects, Tₘ of hybridization of HRa′ of the first polynucleotide and the complementary HRa of the second polynucleotide is between at or about 15° C. and at or about 40° C., such as at or about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or a range defined by any of the foregoing. In some aspects, the annealing and amplification can be performed at any temperature between at or about between at or about 15° C. and at or about 40° C., such as at or about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or a range defined by any of the foregoing.

In some embodiments, the first polynucleotide is modified, as an anchoring site, for anchoring or cross-linking of the polynucleotide to a scaffold, to cellular structures, to other probe polynucleotides and/or to other amplification products, for example, as described in Section V herein. In some aspects, the first polynucleotide is modified at the 5′ end for anchoring or cross-linking. In some embodiments, the first polynucleotide is modified to contain one or more functional groups for anchoring or cross-linking, such as any described herein, for example, a modified nucleotide.

In some embodiments, the first polynucleotide comprises one or more barcode(s). In some embodiments, the one or more barcode(s) include one or more barcode(s) described herein, for example, in Section III.D, or any known nucleic acid barcodes. In some aspects, the one or more barcode(s) in the first polynucleotide can be used as a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the first polynucleotide. In some embodiments, the one or more barcode(s) is located between the 5′ end and HR1 of the first polynucleotide. In some aspects, the first polynucleotide comprises in the 5′ to 3′ direction, 5′ end modification, one or more barcodes, HR1 and HRa′. In some embodiments, the first polynucleotide comprises one barcode. In some embodiments, the first polynucleotide comprises two or more barcodes. In some embodiments, the two or more barcodes are contiguous. In some embodiments, the two or more barcodes are separated by a number of nucleotide sequences.

In some aspects, the first polynucleotide has a length that is sufficient for containing a hybridization region (e.g., HR1) that can hybridize to a target site on the target nucleic acid and a hybridization region (e.g., HRa′) that can hybridize to a hybridization region in the second polynucleotide and can provide the 3′ hydroxyl (—OH) group as a primer for amplification. In some embodiments, HR1 is between about 4 and about 16 nucleotides in length. In some embodiments, HR1 is between about 8 and about 12 nucleotides in length. In some embodiments, HR1 is about 10 nucleotides in length. In some embodiments, HRa′ is between about 2 and about 16 nucleotides in length. In some embodiments, HRa′ is between about 4 and about 8 nucleotides in length. In some embodiments, HRa′ is about 6 nucleotides in length. In some embodiments, HR1 and HRa′ are separated by 0 to 3 nucleotides, such as 0 to 2 nucleotides. In some embodiments, HR1 and HRa′ are separated by 0 nucleotides (e.g., are contiguous). In some embodiments, HR1 and HRa′ are separated by 1 nucleotide. In some embodiments, HR1 and HRa′ are separated by 2 nucleotides.

In some aspects, the first polynucleotide has a length of at least at or about 5 nucleotides, such as at least at or about 10, 15, 20, 25, 30, 35, 40, 45, 50, 60, 70, 80, 90, 100, 110, 120, 130, 140, 150, 160, 170, 180, 190, 200 or more nucleotides, or within a range defined by any of the foregoing. In some embodiments, the first polynucleotide has a length between at or about 5 nucleotides to at or about 200 nucleotides, such as at or about 10, 15, 20, 25, 30, 35, 40, 45, 50, 60, 70, 80, 90, 100, 110, 120, 130, 140, 150, 160, 170, 180, 190, 200 or more, or within a range defined by any of the foregoing. In some embodiments, the first polynucleotide is between about 5 and about 40 nucleotides in length. In some embodiments, the first polynucleotide is between about 10 and about 20 nucleotides in length.

In some embodiments, the first polynucleotide is a DNA molecule. In some embodiments, the first polynucleotide is an RNA molecule and/or comprises one or more ribonucleotides. In some embodiments, the first polynucleotide is a modified nucleic acid molecule or contains modified nucleotides or modified nucleosides, such as methylated nucleotides and nucleotide analogs, uracil, other sugars, and linking groups such as fluororibose and thioate, and nucleotide branches. In some embodiments, the polynucleotide may include non-nucleotide components. In some embodiments, the first polynucleotide may be modified to include N3′-P5′ (NP) phosphoramidate, morpholino phosphorociamidate (MF), locked nucleic acid (LNA), 2′-0-methoxyethyl (MOE), or 2′-fluoro, arabino-nucleic acid (FANA). In some embodiments, the first polynucleotide comprises LNA.

B. Second Polynucleotide

In some aspects, the polynucleotide probe set, such as a three-polynucleotide probe set, includes a second polynucleotide. In some aspects, the second polynucleotide can also be referred to as a “center probe” or a “padlock probe.” FIGS. 1 and 2 depict schematics of an exemplary probe set, including the second polynucleotide (center probe).

Polynucleotides from the probe sets can be hybridized to an analyte, e.g., target nucleic acid, and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary, and include any described herein.

In some embodiments, the second polynucleotide comprises: a target hybridization region (for example, referred to as HR2 in the exemplary schematic depicted in FIGS. 1 and 2) that hybridizes to a complementary (or near-complementary) target site (for example, referred to as HR2′ in the exemplary schematic depicted in FIGS. 1 and 2) in the target nucleic acid; a hybridization region (for example, referred to as HRa in the exemplary schematic depicted in FIGS. 1 and 2) that hybridizes to a hybridization region in the first polynucleotide (for example, referred to as HRa′ in the exemplary schematic depicted in FIGS. 1 and 2); and a different hybridization region (for example, referred to as HRb1 and HRb2 in the exemplary schematic depicted in FIGS. 1 and 2) that hybridizes to a hybridization region in the third polynucleotide (for example, referred to as HRb′ in the exemplary schematic depicted in FIGS. 1 and 2. In some embodiments, the second polynucleotide contains, in the 5′ to 3′ direction, hybridization regions HRb1, HRa and HRb2. In some aspects, HRb1 of the second polynucleotide includes the 5′ end of the second polynucleotide. In some aspects, HRb2 of the second polynucleotide includes the 3′ end of the second polynucleotide. In some embodiments, HRb1 and HRb2 of the second polynucleotide are present at the 5′ and 3′ ends, respectively, and can be ligated in the ligation step, to form a circular sequence that together is complementary to or near-complementary to HRb′ sequence of the third polynucleotide. In some instances, the second polynucleotide comprises a 5′ phosphate.

In some embodiments, the second polynucleotide comprises a target hybridization region (e.g., HR2) that hybridizes to a complementary (or near-complementary) target site (e.g., HR2′) in the target nucleic acid. This region provides a part of the specificity for recognizing and target nucleic acid sequence (e.g., mRNA). In some aspects, the hybridization between HR2/HR2′, together with the hybridization between HR1/HR1′ and HR3/HR3′, provides the specificity for recognition and detection of particular target nucleic acid (e.g., mRNA).

In some embodiments, the length of each of HR2 of the second polynucleotide and the complementary HR2′ on the target nucleic acid, is about 4 to about 40 nucleotides, such as about 4 to about 16 nucleotides or about 8 to about 12 nucleotides. In some aspects, the length of each of HR2 and HR2′ is about 8 nucleotides. In some aspects, the length of each of HR2 and HR2′ is about 9 nucleotides. In some aspects, the length of each of HR2 and HR2′ is about 10 nucleotides. In some aspects, the length of each of HR2 and HR2′ is about 11 nucleotides. In some aspects, the length of each of HR2 and HR2′ is about 12 nucleotides. In some aspects, the length of each of HR2 and HR2′ is about 13 nucleotides. In some aspects, the length of each of HR2 and HR2′ is about 14 nucleotides. In some aspects, the length of each of HR2 and HR2′ is about 15 nucleotides. In some aspects, the length of each of HR2 and HR2′ is about 16 nucleotides. In some aspects, the length of each of HR2 and HR2′ is about 17 nucleotides. In some aspects, the length of each of HR2 and HR2′ is about 18 nucleotides. In some aspects, the length of each of HR2 and HR2′ is about 19 nucleotides. In some aspects, the length of each of HR2 and HR2′ is about 20 nucleotides. In some aspects, the length of each of HR2 and HR2′ is fewer than 8 nucleotides. In some aspects, the length of each of HR2 and HR2′ is fewer than 9 nucleotides. In some aspects, the length of each of HR2 and HR2′ is fewer than 10 nucleotides. In some aspects, the length of each of HR2 and HR2′ is fewer than 11 nucleotides. In some aspects, the length of each of HR2 and HR2′ is fewer than 12 nucleotides. In some aspects, the length of each of HR2 and HR2′ is fewer than 13 nucleotides. In some aspects, the length of each of HR2 and HR2′ is fewer than 14 nucleotides. In some aspects, the length of each of HR2 and HR2′ is fewer than 15 nucleotides. In some aspects, the length of each of HR2 and HR2′ is fewer than 16 nucleotides. In some aspects, the length of each of HR2 and HR2′ is fewer than 17 nucleotides. In some aspects, the length of each of HR2 and HR2′ is fewer than 18 nucleotides. In some aspects, the length of each of HR2 and HR2′ is fewer than 19 nucleotides. In some aspects, the length of each of HR2 and HR2′ is fewer than 20 nucleotides.

In some embodiments, the second polynucleotide comprises two separate hybridization regions, each at the 5′ and 3′ end of the second polynucleotide (e.g., referred to as HRb1 and HRb2 in the exemplary schematic depicted in FIGS. 1 and 2), that collectively can hybridize to (e.g., is complementary or near-complementary to) a hybridization region of the third polynucleotide (e.g., also called a “splint” or a “splint template” and referred to as HRb′ in the exemplary schematic depicted in FIGS. 1 and 2). In some aspects, the second polynucleotide is a padlock probe. In some aspects, a padlock probes is a linear circularizable oligonucleotide which has free 5′ and 3′ ends which are available for ligation, to result in the adoption of a circular conformation. In some aspects, for circularization (ligation) to occur, the padlock probe has a free 5′ phosphate group, for example, at the 5′ end of the second polynucleotide comprising a HRb1. To allow the juxtaposition of the ends of the padlock probe for ligation, the padlock probe is designed to have at its 5′ and 3′ ends regions of complementarity to a splint (e.g., HRb′ present on the third polynucleotide). In some aspects, the hybridization regions allow specific binding of the padlock probe to its target sequence by virtue of hybridization to the splint. In some aspects, the ligation may be direct or indirect. In some instances, the ends of the padlock probe may be ligated directly to each other or they may be ligated to an intervening nucleic acid molecule/sequence of nucleotides. In some embodiments, HRb1 and HRb2 of the padlock probe may be complementary to adjacent, or contiguous, regions in the third polynucleotide, or complementary to non-adjacent (non-contiguous) regions of the third polynucleotide, in which case, for ligation to occur, the “gap” between the two ends of the hybridized padlock probe is filled by an intervening molecule/sequence.

In some embodiments, the second polynucleotide (center probe or padlock probe) is ligated to circularize, with HRb′ of the third polynucleotide as a template or a splint for ligation. In some aspects, HRb1 is present at the 5′ end of the second polynucleotide, and HRb2 is present at the 3′ end of the second polynucleotide. In some embodiments, HRb′ region of the third polynucleotide, which can hybridize to HRb1 and HRb2 of the second polynucleotide, acts as a template or a splint, to guide ligation of the 5′ end containing HRb1 and the 3′ end containing HRb2, to form a circular sequence from the second polynucleotide. In some embodiments, the ligated HRb1 and HRb2 sequence (e.g., in circularized second polynucleotide) together is complementary to or near-complementary to HRb′ sequence of the third polynucleotide. In some aspects, the ligation step can also include gap-filling, in cases in which there may be a gap (non-contiguous nucleic acids) between the complementarity of the 5′ end containing HRb1 and the 3′ end containing HRb2, when hybridizing to HRb′ of the third polynucleotide.

In some embodiments, HRb1 and HRb2 are approximately equal in length. In some embodiments, HRb1 and HRb2 are different in length. In some embodiments, HRb1 is longer than HRb2, for example by at or about 1, 2, 3, 4, 5, 6, 7, 8, 9 or 10 nucleotides. In some embodiments, HRb2 is longer than HRb1, for example by at or about 1, 2, 3, 4, 5, 6, 7, 8, 9 or 10 nucleotides. In some embodiments, the length of one or each of HRb1 and HRb2 of the second polynucleotide is at least 1 nucleotide, such as at least 2 nucleotides. In some aspects, the length of one or each of HRb1 and HRb2 is about 3 to about 40 nucleotides, such as about 4 to about 20 nucleotides. In some aspects, the length of one or each of HRb1 and HRb2 is about 3 nucleotides. In some aspects, the length of one or each of HRb1 and HRb2 is about 4 nucleotides. In some aspects, the length of one or each of HRb1 and HRb2 is about 5 nucleotides. In some aspects, the length of one or each of HRb1 and HRb2 is about 6 nucleotides. In some aspects, the length of one or each of HRb1 and HRb2 is about 7 nucleotides. In some aspects, the length of one or each of HRb1 and HRb2 is about 8 nucleotides. In some aspects, the length of one or each of HRb1 and HRb2 is about 9 nucleotides. In some aspects, the length of one or each of HRb1 and HRb2 is about 10 nucleotides. In some aspects, the length of one or each of HRb1 and HRb2 is about 11 nucleotides. In some aspects, the length of one or each of HRb1 and HRb2 is about 12 nucleotides. In some aspects, the length of one or each of HRb1 and HRb2 is about 13 nucleotides. In some aspects, the length of one or each of HRb1 and HRb2 is about 14 nucleotides. In some aspects, the length of one or each of HRb1 and HRb2 is about 15 nucleotides. In some aspects, the length of one or each of HRb1 and HRb2 is about 16 nucleotides. In some aspects, the length of one or each of HRb1 and HRb2 is about 17 nucleotides. In some aspects, the length of one or each of HRb1 and HRb2 is about 18 nucleotides. In some aspects, the length of one or each of HRb1 and HRb2 is about 19 nucleotides. In some aspects, the length of one or each of HRb1 and HRb2 is about 20 nucleotides. In some aspects, the length of one or each of HRb1 and HRb2 is fewer than 5 nucleotides. In some aspects, the length of one or each of HRb1 and HRb2 is fewer than 6 nucleotides. In some aspects, the length of one or each of HRb1 and HRb2 is fewer than 7 nucleotides. In some aspects, the length of one or each of HRb1 and HRb2 is fewer than 8 nucleotides. In some aspects, the length of one or each of HRb1 and HRb2 is fewer than 9 nucleotides. In some aspects, the length of one or each of HRb1 and HRb2 is fewer than 10 nucleotides. In some aspects, the length of one or each of HRb1 and HRb2 is fewer than 11 nucleotides. In some aspects, the length of one or each of HRb1 and HRb2 is fewer than 12 nucleotides. In some aspects, the length of one or each of HRb1 and HRb2 is fewer than 13 nucleotides. In some aspects, the length of one or each of HRb1 and HRb2 is fewer than 14 nucleotides. In some aspects, the length of one or each of HRb1 and HRb2 is fewer than 15 nucleotides. In some aspects, the length of one or each of HRb1 and HRb2 is fewer than 16 nucleotides. In some aspects, the length of one or each of HRb1 and HRb2 is fewer than 17 nucleotides. In some aspects, the length of one or each of HRb1 and HRb2 is fewer than 18 nucleotides. In some aspects, the length of one or each of HRb1 and HRb2 is fewer than 19 nucleotides. In some aspects, the length of one or each of HRb1 and HRb2 is fewer than 20 nucleotides.

In some embodiments, the second polynucleotide comprises one or more barcode(s). In some embodiments, the one or more barcode(s) include one or more barcode(s) described herein, for example, in Section III.D, or any known nucleic acid barcodes. In some aspects, the one or more barcode(s) in the second polynucleotide permits detection and identification of the second polynucleotide or an amplification product of the second polynucleotide. In some aspects, the one or more barcode(s) is located between the 5′ and 3′ ends of the second polynucleotide. In some embodiments, the one or more barcode(s) is located between HRb1 and HRb2 of the second polynucleotide. In some aspects, the one or more barcode(s) is located between HRb1 and HRa of the second polynucleotide. In some aspects, the second polynucleotide comprises in the 5′ to 3′ direction, hybridization regions HRb1, barcode, HRa and HRb2. In some embodiments, the second polynucleotide comprises one barcode. In some embodiments, the second polynucleotide comprises two or more barcodes. In some embodiments, the two or more barcodes are contiguous. In some embodiments, the two or more barcodes are separated by a number of nucleotide sequences.

In some embodiments, the second polynucleotide is amplified, for example by rolling circle amplification (RCA). In some aspects, the RCA is performed after ligation of the 5′ and 3′ ends of the second polynucleotide, for example, as guided by HRb′ present on the third polynucleotide. In some aspects, the ligation results in a circular polynucleotide, from which RCA can begin in the presence of a primer containing a 3′ hydroxyl (—OH) group. In some aspects, the RCA results in amplification of the second polynucleotide, for example, as a concatenated product of copies of the second polynucleotide. In some aspects, one or more portions of the amplification product (e.g., amplicon), such as one or more barcode(s), can be detected in the detection step.

In some embodiments, the second polynucleotide comprises a hybridization region that hybridizes to a complementary (or near-complementary) region in the first polynucleotide of the probe set. In some aspects, HRa of the second polynucleotide can hybridize to HRa′ of the second polynucleotide (with reference to HR regions as depicted in FIGS. 1 and 2). In some aspects, as described above, the first polynucleotide contains a 3′ hydroxyl (—OH) group which serves as a primer and the 3′-OH for beginning rolling circle amplification (RCA) of the second polynucleotide, after ligation of 3′ and 5′ ends of the second polynucleotides to circularize the second polynucleotide. In some instances, HRa′ and HRa hybridization interaction is not used as a splint for ligation of the second polynucleotide. Thus, in some embodiments, HRa′ and HRa interaction is utilized for specificity of the probe set for the target nucleic acid, but not for ligation of the second polynucleotide (e.g., the second polynucleotide does not comprise a nick or gap in HRa).

In some embodiments, the length of each of HRa of the second polynucleotide and the complementary HRa′ of the first polynucleotide is at least 1 nucleotide, such as at least 2 nucleotides. In some aspects, the length of each of HRa and HRa′ is about 3 to about 40 nucleotides, such as about 4 to about 20 nucleotides.

In some embodiments, the melting temperature (Tₘ) of the Tₘ of the hybridization between HRb1 and HRb2 of the second polynucleotide and the splint (e.g., HRb′) of the third polynucleotide is lower than the Tₘ of hybridization of HR between the probe polynucleotides and target site on the target nucleic acid (e.g., HR1/HR1′, HR2/HR2′ or HR3/HR3′). In some aspects, such differences in Tₘ permits the hybridization between the probe polynucleotides and target site on the target nucleic acid while reducing or minimizing the inter-polynucleotide probe hybridization (e.g., between HRb1-HRb2/HRb′) during the hybridization step. In some aspects, such differences in Tₘ permits the hybridization between the probe polynucleotides and target site on the target nucleic acid to be maintained during the ligation step, during amplification of the second polynucleotide by rolling circle amplification, and/or other subsequent steps. In some aspects, the ligation step can be performed at a temperature that is lower than the Tₘ of hybridization of HRs between the probe polynucleotides and target site on the target nucleic acid, for example at a temperature required for the hybridization of the 5′ and 3′ ends (e.g., comprising HRb1-HRb2) and the splint (e.g., HRb′) and ligation of the 5′ and 3′ ends of the second polynucleotide. In some aspects, Tₘ of hybridization of the hybridization between HRb1-HRb2 of the second polynucleotide and the complementary HRb′ of the third polynucleotide is between at or about 15° C. and at or about 40° C., such as at or about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or a range defined by any of the foregoing. In some aspects, the hybridization with the splint and ligation can be performed at any temperature between at or about between at or about 15° C. and at or about 40° C., such as at or about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or a range defined by any of the foregoing.

In some aspects, the second polynucleotide has a length that is sufficient for containing 5′ and 3′ hybridization regions for hybridizing to a splint on the third polynucleotide (e.g., HRb1 and HRb2), a hybridization region (e.g., HR2) that can hybridize to a target site on the target nucleic acid and a hybridization region (e.g., HRa) that can hybridize to a hybridization region in the first polynucleotide.

In some embodiments, HR2 is between about 4 and about 16 nucleotides in length. In some embodiments, HR2 is between about 8 and about 12 nucleotides in length. In some embodiments, HR2 is about 10 nucleotides in length. In some embodiments, HRa is between about 2 and about 16 nucleotides in length. In some embodiments, HRa is between about 4 and about 8 nucleotides in length. In some embodiments, HRa is about 6 nucleotides in length. In some embodiments, HRb1 is between about 1 and about 15 nucleotides in length. In some embodiments, HRb2 is between about 1 and about 15 nucleotides in length. In some embodiments, HRb1 and HRb2 combined is between about 4 and about 16 nucleotides in length. In some embodiments, HRb1 and HRb2 combined is between about 8 and about 12 nucleotides in length, In some embodiments, HRb1 and HRb2 combined is about 10 nucleotides in length. In some embodiments, HRa and HR2 are separated by 0 to 3 nucleotides, such as 0 to 2 nucleotides. In some embodiments, HRa and HR2 are separated by 0 nucleotides (e.g., are contiguous). In some embodiments, HRa and HR2 are separated by 1 nucleotide. In some embodiments, HRa and HR2 are separated by 2 nucleotides. In some embodiments, HR2 and HRb2 are separated by 0 to 3 nucleotides, such as 0 to 2 nucleotides. In some embodiments, HR2 and HRb2 are separated by 0 nucleotides (e.g., are contiguous). In some embodiments, HR2 and HRb2 are separated by 1 nucleotide. In some embodiments, HR2 and HRb2 are separated by 2 nucleotides. In some embodiments, HR2 and HRb2 are separated by fewer than 2 nucleotides.

In some aspects, the second polynucleotide may be of any suitable length to act as a template for amplification, such as rolling circle amplification. In some embodiments, the second polynucleotide, prior to circularization, may have a length of between at or about 50 and at or about 150 nucleotides, of between at or about 60 and at or about 120 nucleotides, or of between at or about 70 and at or about 100 nucleotides. For example, the second polynucleotide may have, for instance, a length of, of at least, or of at most 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200 or more nucleotides, or within any range defined by any of the foregoing. In some aspects, the second polynucleotide prior to circularization has a length of at least at or about 15 nucleotides, such as at least at or about 20, 25, 30, 35, 40, 45, 50, 60, 70, 80, 90, 100, 110, 120, 130, 140, 150, 160, 170, 180, 190, 200 or more nucleotides, or within a range defined by any of the foregoing. In some embodiments, the second polynucleotide prior to circularization has a length between at or about 15 nucleotides to at or about 200 nucleotides, such as at or about 20, 25, 30, 35, 40, 45, 50, 60, 70, 80, 90, 100, 110, 120, 130, 140, 150, 160, 170, 180, 190, 200 or more nucleotides, or within a range defined by any of the foregoing. In some embodiments, the second polynucleotide prior to circularization is between about 15 and about 60 nucleotides in length. In some embodiments, the second polynucleotide prior to circularization is between about 25 and about 45 nucleotides in length. In some embodiments, the second polynucleotide prior to circularization is about 35 nucleotides in length.

In some embodiments, the second polynucleotide is a DNA molecule. In some embodiments, the second polynucleotide is an RNA molecule or comprises ribonucleotides. In some embodiments, the second polynucleotide is a modified nucleic acid molecule or contains modified nucleotides or modified nucleosides, such as methylated nucleotides and nucleotide analogs, uracil, other sugars, and linking groups such as fluororibose and thioate, and nucleotide branches. In some embodiments, the polynucleotide may include non-nucleotide components. In some embodiments, the second polynucleotide may be modified to include N3′-P5′ (NP) phosphoramidate, morpholino phosphorociamidate (MF), locked nucleic acid (LNA), 2′-0-methoxyethyl (MOE), or 2′-fluoro, arabino-nucleic acid (FANA). In some embodiments, the second polynucleotide comprises LNA. In some aspects, the modification can be used as an anchoring site to anchor the amplified product to the scaffold, other probe polynucleotides, other amplification products and/or to cellular structures, e.g., as described in Section V herein.

C. Third Polynucleotide

In some aspects, the polynucleotide probe set, such as a three-polynucleotide probe set, includes a third polynucleotide. In some aspects, the third polynucleotide can also be referred to as a “right probe.” FIGS. 1 and 2 depict schematics of an exemplary probe set, including the third polynucleotide (right probe).

In some embodiments, the third polynucleotide comprises: a target hybridization region (for example, referred to as HR3 in the exemplary schematic depicted in FIGS. 1 and 2) that hybridizes to a complementary (or near-complementary) target site (for example, referred to as HR3′ in the exemplary schematic depicted in FIGS. 1 and 2) in the target nucleic acid; and a different hybridization region (for example, referred to as HRb′ in the exemplary schematic depicted in FIGS. 1 and 2) that hybridizes to hybridization regions in the second polynucleotide (for example, referred to as HRb1 and HRb2 in the exemplary schematic depicted in FIGS. 1 and 2). In some embodiments, the third polynucleotide contains, in the 5′ to 3′ direction, hybridization regions HRb′ and HR3.

In some embodiments, the third polynucleotide comprises a target hybridization region (e.g., HR3) that hybridizes to a complementary (or near-complementary) target site (e.g., HR3′) in the target nucleic acid. This region provides a part of the specificity for recognizing and target nucleic acid sequence (e.g., mRNA). In some aspects, the hybridization between HR3/HR3′, together with the hybridization between HR1/HR1′ and HR2/HR2′, provides the specificity for recognition and detection of particular target nucleic acid (e.g., mRNA).

In some embodiments, the length of each of HR3 of the third polynucleotide and the complementary HR3′ on the target nucleic acid, is about 4 to about 40 nucleotides, such as about 4 to about 16 nucleotides or about 8 to about 12 nucleotides. In some aspects, the length of each of HR3 and HR3′ is about 8 nucleotides. In some aspects, the length of each of HR3 and HR3′ is about 9 nucleotides. In some aspects, the length of each of HR3 and HR3′ is about 10 nucleotides. In some aspects, the length of each of HR3 and HR3′ is about 11 nucleotides. In some aspects, the length of each of HR3 and HR3′ is about 12 nucleotides. In some aspects, the length of each of HR3 and HR3′ is about 13 nucleotides. In some aspects, the length of each of HR3 and HR3′ is about 14 nucleotides. In some aspects, the length of each of HR3 and HR3′ is about 15 nucleotides. In some aspects, the length of each of HR3 and HR3′ is about 16 nucleotides. In some aspects, the length of each of HR3 and HR3′ is about 17 nucleotides. In some aspects, the length of each of HR3 and HR3′ is about 18 nucleotides. In some aspects, the length of each of HR3 and HR3′ is about 19 nucleotides. In some aspects, the length of each of HR3 and HR3′ is about 20 nucleotides. In some aspects, the length of each of HR3 and HR3′ is fewer than 8 nucleotides. In some aspects, the length of each of HR3 and HR3′ is fewer than 9 nucleotides. In some aspects, the length of each of HR3 and HR3′ is fewer than 10 nucleotides. In some aspects, the length of each of HR3 and HR3′ is fewer than 11 nucleotides. In some aspects, the length of each of HR3 and HR3′ is fewer than 12 nucleotides. In some aspects, the length of each of HR3 and HR3′ is fewer than 13 nucleotides. In some aspects, the length of each of HR3 and HR3′ is fewer than 14 nucleotides. In some aspects, the length of each of HR3 and HR3′ is fewer than 15 nucleotides. In some aspects, the length of each of HR3 and HR3′ is fewer than 16 nucleotides. In some aspects, the length of each of HR3 and HR3′ is fewer than 17 nucleotides. In some aspects, the length of each of HR3 and HR3′ is fewer than 18 nucleotides. In some aspects, the length of each of HR3 and HR3′ is fewer than 19 nucleotides. In some aspects, the length of each of HR3 and HR3′ is fewer than 20 nucleotides.

In some embodiments, the third polynucleotide comprises a hybridization region that hybridizes to a complementary (or near-complementary) region in the second polynucleotide of the probe set. In some aspects, HRb′ of the third polynucleotide can hybridize to a combined region of HRb1 and HRb2 of the second polynucleotide (with reference to HR regions as depicted in FIGS. 1 and 2). In some embodiments the split hybridization region (e.g., discontinuous hybridization region having a nick or a gap) formed by HRb1 and HRb2 comprises a nick between the 5′ end of HRb1 and the 3′ end of HRb2 when the split hybridization region is hybridized to HRb′. In some aspects, HRb′ of the third polynucleotide acts as a splint for ligation of the ends of the second polynucleotide. In some aspects, HRb′ of the third polynucleotide guides the aligning (e.g., juxtaposition) of HRb1 and HRb2 of the second polynucleotide for ligation. In some cases, the 5′ end and the 3′ end of the second polynucleotide are aligned without a gap, and the ends are ligated directly. In some embodiments, a gap is present the 5′ end (comprising HRb1) of and the 3′ end (comprising HRb2) of the second polynucleotide, when hybridized and aligned with HRb′ of the third polynucleotide. In some embodiments, the split hybridization region formed by HRb1 and HRb2 comprises a gap between the 5′ end of HRb1 and the 3′ end of HRb2 when the split hybridization region hybridized to HRb′. In some embodiments, the gap is between about 1 and about 5 nucleotides in length, such as at or about 1, 2, 3, 4 or 5, or more nucleotides in length. In some aspects, gap-filling can be performed prior to ligation.

In some embodiments, the length of HRb′ of the third polynucleotide is at least 2 nucleotides, such as at least 3 nucleotides. In some aspects, the length of HRb′ is in sufficient length to provide a template or a splint for ligation of the 5′ and 3′ ends of the second polynucleotide. In some aspects, the length of HRb′ is about 5 nucleotides. In some aspects, the length of HRb′ is about 6 nucleotides. In some aspects, the length of HRb′ is about 7 nucleotides. In some aspects, the length of HRb′ is about 8 nucleotides. In some aspects, the length of HRb′ is about 9 nucleotides. In some aspects, the length of HRb′ is about 10 nucleotides. In some aspects, the length of HRb′ is about 11 nucleotides. In some aspects, the length of HRb′ is about 12 nucleotides. In some aspects, the length of HRb′ is about 13 nucleotides. In some aspects, the length of HRb′ is about 14 nucleotides. In some aspects, the length of HRb′ is about 15 nucleotides. In some aspects, the length of HRb′ is about 16 nucleotides. In some aspects, the length of HRb′ is about 17 nucleotides. In some aspects, the length of HRb′ is about 18 nucleotides. In some aspects, the length of HRb′ is about 19 nucleotides. In some aspects, the length of HRb′ is about 20 nucleotides. In some aspects, the length of HRb′ is fewer than 5 nucleotides. In some aspects, the length of HRb′ is fewer than 6 nucleotides. In some aspects, the length of HRb′ is fewer than 7 nucleotides. In some aspects, the length of HRb′ is fewer than 8 nucleotides. In some aspects, the length of HRb′ is fewer than 9 nucleotides. In some aspects, the length of HRb′ is fewer than 10 nucleotides. In some aspects, the length of HRb′ is fewer than 11 nucleotides. In some aspects, the length of HRb′ is fewer than 12 nucleotides. In some aspects, the length of HRb′ is fewer than 13 nucleotides. In some aspects, the length of HRb′ is fewer than 14 nucleotides. In some aspects, the length of HRb′ is fewer than 15 nucleotides. In some aspects, the length of HRb′ is fewer than 16 nucleotides. In some aspects, the length of HRb′ is fewer than 17 nucleotides. In some aspects, the length of HRb′ is fewer than 18 nucleotides. In some aspects, the length of HRb′ is fewer than 19 nucleotides. In some aspects, the length of HRb′ is fewer than 20 nucleotides.

In some embodiments, the melting temperature (Tₘ) of the hybridization between HRb′ of the third polynucleotide and the complementary HRb1-HRb2 of the second polynucleotide is lower than the Tₘ of hybridization of HR between the probe polynucleotides and target site on the target nucleic acid (e.g., HR1/HR1′, HR2/HR2′ or HR3/HR3′). In some aspects, such differences in Tₘ permits the hybridization between the probe polynucleotides and target site on the target nucleic acid while reducing or minimizing the inter-polynucleotide probe hybridization (e.g., between HRb′ and HRb1-HRb2) during the hybridization step. In some aspects, such differences in Tₘ permits the hybridization between the probe polynucleotides and target site on the target nucleic acid to be maintained during the ligation of the 5′ and 3′ ends of the second polynucleotide, gap filling, amplification of the second polynucleotide by rolling circle amplification, and/or other subsequent steps. In some aspects, the ligation, gap filling, amplification and/or subsequent steps can be performed at a temperature that is lower than the Tₘ of hybridization of HRs between the probe polynucleotides and target site on the target nucleic acid, at a temperature required for the ligation and/or gap filling. In some aspects, Tₘ of hybridization of HRb′ of the third polynucleotide and the complementary HRb1-HRb2 of the second polynucleotide is between at or about 15° C. and at or about 40° C., such as at or about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or a range defined by any of the foregoing. In some aspects, the ligation and/or gap filling can be performed at any temperature between at or about between at or about 15° C. and at or about 40° C., such as at or about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., or a range defined by any of the foregoing.

In some embodiments, the third polynucleotide is modified, as an anchoring site, for anchoring or cross-linking of the polynucleotide to a scaffold, to cellular structures, to other probe polynucleotides and/or to other amplification products, for example, as described in Section V herein. In some aspects, the third polynucleotide is modified at the 3′ end for anchoring or cross-linking. In some embodiments, the third polynucleotide is modified to contain one or more functional groups for anchoring or cross-linking, such as any described herein, for example, a modified nucleotide.

In some embodiments, the third polynucleotide comprises one or more barcode(s). In some embodiments, the one or more barcode(s) include one or more barcode(s) described herein, for example, in Section III.D, or any known nucleic acid barcodes. In some aspects, the one or more barcode(s) in the third polynucleotide can be used as a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the third polynucleotide. In some embodiments, the one or more barcode(s) is located between HR3 and the 3′ end of the third polynucleotide. In some aspects, the third polynucleotide comprises in the 5′ to 3′ direction, HRb′, HR3, one or more barcodes, and 3′ end. In some embodiments, the third polynucleotide comprises one barcode. In some embodiments, the third polynucleotide comprises two or more barcodes. In some embodiments, the two or more barcodes are contiguous. In some embodiments, the two or more barcodes are separated by a number of nucleotide sequences.

In some aspects, the third polynucleotide has a length that is sufficient for containing a hybridization region (e.g., HR3) that can hybridize to a target site on the target nucleic acid and a hybridization region (e.g., HRb′) that can hybridize to hybridization regions (e.g., HRb1-HRb2) in the second polynucleotide and can provide a template or a splint for ligation of the 5′ and 3′ ends of the second polynucleotide. In some aspects, the third polynucleotide can provide a DNA template for efficient ligation of the 5′ and 3′ ends of the second polynucleotide. In some embodiments, HR3 is between about 4 and about 16 nucleotides in length. In some embodiments, HR3 is between about 8 and about 12 nucleotides in length. In some embodiments, HR3 is about 10 nucleotides in length. In some embodiments, HRb′ is between about 4 and about 16 nucleotides in length. In some embodiments, HRb′ is between about 8 and about 12 nucleotides in length. In some embodiments, HRb′ is about 10 nucleotides in length. In some embodiments, HRb′ and HRb3 are separated by 0 to 3 nucleotides, such as 0 to 2 nucleotides. In some embodiments, HRb′ and HRb3 are separated by 0 nucleotides (e.g., are contiguous). In some embodiments, HRb′ and HRb3 are separated by 1 nucleotide. In some embodiments, HRb′ and HRb3 are separated by 2 nucleotides. In some embodiments, HRb′ and HRb3 are separated by fewer than 2 nucleotides.

In some aspects, the third polynucleotide has a length of at least at or about 5 nucleotides, such as at least at or about 10, 15, 20, 25, 30, 35, 40, 45, 50, 60, 70, 80, 90, 100, 110, 120, 130, 140, 150, 160, 170, 180, 190, 200 or more nucleotides, or within a range defined by any of the foregoing. In some embodiments, the third polynucleotide has a length between at or about 5 nucleotides to at or about 200 nucleotides, such as at or about 10, 15, 20, 25, 30, 35, 40, 45, 50, 60, 70, 80, 90, 100, 110, 120, 130, 140, 150, 160, 170, 180, 190, 200 or more nucleotides, or within a range defined by any of the foregoing. In some embodiments, the third polynucleotide is between about 5 and about 40 nucleotides in length. In some embodiments, the third polynucleotide is between about 10 and about 20 nucleotides in length.

In some embodiments, the third polynucleotide is a DNA molecule. In some embodiments, the third polynucleotide is an RNA molecule, or comprises ribonucleotides. In some embodiments, the third polynucleotide is a modified nucleic acid molecule or contains modified nucleotides or modified nucleosides, such as methylated nucleotides and nucleotide analogs, uracil, other sugars, and linking groups such as fluororibose and thioate, and nucleotide branches. In some embodiments, the polynucleotide may include non-nucleotide components. In some embodiments, the third polynucleotide may be modified to include N3′-P5′ (NP) phosphoramidate, morpholino phosphorociamidate (MF), locked nucleic acid (LNA), 2′-0-methoxyethyl (MOE), or 2′-fluoro, arabino-nucleic acid (FANA). In some embodiments, the third polynucleotide is or comprises LNA.

D. Barcoding

In some aspects, one or more of the polynucleotide probe(s) includes one or more barcode(s). In some aspects, the one or more barcode is included in the amplified polynucleotide (e.g., present in the second polynucleotide, also called the center probe or the padlock probe). In some embodiments, the one or more barcode(s) is on a polynucleotide probe that is not amplified (e.g., the first polynucleotide and/or third polynucleotide) and can provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some aspects, one or more of the polynucleotide probe(s) includes one or more barcode(s), e.g., at least two, three, four, five, six, seven, eight, nine, ten, or more barcodes. Barcodes can spatially-resolve molecular components found in biological samples, for example, within a cell or a tissue sample. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some embodiments, barcode sequences are detected for identification of other molecules, including nucleic acid molecules (DNA or RNA) longer than the barcode sequences themselves, as opposed to direct sequencing of the longer nucleic acid molecules.

In some embodiments, one or more of the barcodes disclosed herein can be correlated with the sequence complementary to the analyte (e.g., target nucleic acid), and thus a particular analyte. A number (n) of analytes (e.g., target nucleic acid) can be examined by introducing (n) different sequences complementary to an analyte/barcode pluralities to the sample. In some embodiments, sequences complementary to an analyte can be used in multiplexed methods to analyze 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, or more analytes.

In some embodiments, methods provided herein include analyzing the one or more barcode(s) (e.g., primary and/or secondary barcode sequences) of the polynucleotide probe set using multiplexed spatial imaging. In some embodiments, analyzing the barcode of the amplicons includes employing RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In some embodiments, the methods provided herein include analyzing the barcode of the composite padlock or circular probes by sequential hybridization and detection with a plurality of labelled probes (e.g., detection oligonucleotides). A variety of light-based sequencing technologies are known in the art. See, e.g., Landegren et al., Genome Res. 8:769-76 (1998); Kwok, Pharmocogenomics 1:95-100 (2000); and Shi, Clin. Chem. 47:164-172 (2001).

In some aspects, the barcode provides information for identification of the target nucleic acid. In some aspects, the terms “barcode” or “one or more barcodes” refers a label, or identifier, that conveys or is capable of conveying information (e.g., information about a target nucleic acid in a sample or a molecule such as a probe polynucleotide), such as a nucleic acid sequence that is used to identify, e.g., a single cell or a subpopulation of cells or a single target nucleic acid or a subset of target nucleic acids. Barcodes can be linked to a target nucleic acid of interest during amplification and used to trace back the amplicon to, for example, the cell and/or position in a tissue from which the target nucleic acid originated. A barcode can be added to a target nucleic acid of interest during amplification by carrying out amplification with a polynucleotide that contains a region including the barcode and a region that is complementary to the target nucleic acid such that the barcode is incorporated into the final amplified target nucleic acid product (i.e., amplicon). A particular barcode can be unique relative to other barcodes. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some embodiments, the first, second or third polynucleotides may include a common sequence shared among a plurality of polynucleotide probe sets where each set targets a different target nucleic acid.

In some embodiments, the barcode sequences comprise 4^{N} complexity given a sequencing read of N bases, and a much shorter sequencing read may be required for molecular identification by non-barcode sequencing methods such as direct sequencing of an RNA target or a cDNA. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes are detected, rather than endogenous sequences, which can be an efficient read-out in terms of information per cycle of sequencing. Because the barcode sequences are pre-determined, they can also be designed to feature error detection and correction mechanisms.

In some aspects, one or more of the polynucleotide probe(s) includes one or more barcode(s). In some aspects, at least two, three, four, five, six, seven, eight, nine, 10, or more barcodes are included in the padlock or circular probe formed of the plurality of polynucleotides.

The barcode sequencing methods disclosed herein can be applied to any sample from which spatial information is of interest. For example, the sample can be a biological sample, including a cell, a tissue, and a cellular matrix. Depending on the application, the biological sample can also be whole blood, serum, plasma, mucosa, saliva, cheek swab, urine, stool, cells, tissue, bodily fluid or a combination thereof.

Barcodes can spatially-resolve molecular components found in biological samples, for example, within a cell or a tissue sample. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, the provided polynucleotide probes (e.g., one or more of the set of polynucleotide probes) are primary probes that bind to a target nucleic acid and comprise one or more primary barcode sequences. In some embodiments, an amplification product (e.g., an RCA product) comprises multiple copies of the one or more primary barcode sequences or complementary sequences thereof, and the amplification product is detected using one or more detection probes (e.g., a detectably labeled oligonucleotides such as fluorescent oligonucleotides) that hybridize to the one or more primary barcode sequences or complementary sequences thereof.

In some embodiments, the method further comprises using one or more secondary probes that hybridize to the one or more primary barcode sequences or complementary sequences thereof on one or more primary probes (e.g., one or more of the set of the first, second, and third polynucleotide probes disclosed herein) that bind to a target nucleic acid such as an mRNA. In some embodiments, the one or more secondary probes hybridize to an amplification product (e.g., an RCA product) comprising multiple copies of the one or more primary barcode sequences or complementary sequences thereof. In some embodiments, the one or more secondary probes comprise one or more secondary barcode sequences and are detected using one or more detection probes (e.g., a detectably labeled oligonucleotides such as fluorescent oligonucleotides) that hybridize to the one or more second barcode sequences or complementary sequences thereof.

In any of the embodiments, barcodes (e.g., primary and/or secondary barcode sequences) can be analyzed (e.g., detected or sequenced) using any suitable methods or techniques, including those described herein, for example, in Section IV, and such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, targeted in situ sequencing, fluorescent in situ sequencing (FISSEQ), or spatially-resolved transcript amplicon readout mapping (STARmap). In some embodiments, the methods provided herein include analyzing the barcodes by sequential hybridization and detection with a plurality of labelled probes (e.g., detection oligonucleotides). In some embodiments, detection of the barcodes includes amplifying the barcode sequences in situ, as described herein, for example, in Section IV.D. In some examples, the amplification can be performed using a non-enzymatic amplification method. In some embodiments, the amplification of the signal refers to an amplified signal associated with the barcode, which may include hybridization chain reaction (HCR) directly or indirectly on the one or more barcode sequences, linear oligonucleotide hybridization chain reaction (LO-HCR) directly or indirectly on the one or more barcode sequences, primer exchange reaction (PER) directly or indirectly on the one or more barcode sequences, assembly of branched structures directly or indirectly on the one or more barcode sequences, hybridization of a plurality of detectable probes directly or indirectly on the one or more barcode sequences, or any combination thereof.

E. Target Sites (e.g., Hybridization Regions) on Target Nucleic Acids

The three polynucleotide probes can each hybridize to an analyte, such as a target nucleic acid (e.g., mRNA), via different target sites. In some embodiments, target hybridization regions are selected to provide selective or unique binding to a particular target site, relative to other nucleic acids present in the cell. The target nucleic acid contains target sites HR1′, HR2′, and HR3′, which can hybridize to target hybridization regions HR1 of the first polynucleotide, HR2 of the second polynucleotide, and HR3 of the third polynucleotide, respectively (with reference to the HRs depicted in the exemplary schematic set forth in FIGS. 1 and 2). The HR1′, HR2′ and HR3′ regions are designated in sequential configuration, in the order of HR1′, HR2′ and HR3′ in the 3′ to 5′ direction of the target nucleic acid. The HR1, HR2 and HR3 regions in the first polynucleotide, the second polynucleotide and the third polynucleotide are complementary to, or are sufficiently complementary to the HR1, HR2 and HR3, respectively, of the target nucleic acid. Following hybridization, the first polynucleotide (e.g., left probe), the second polynucleotide (e.g., center probe) and the third polynucleotide (e.g., right probe) will hybridize, e.g., in a sequential configuration, in the 3′ to 5′ direction of the target nucleic acid. In some embodiments, the one or more polynucleotide probe sets, which can include three polynucleotide probes, bind to a different region of the target nucleic acid (e.g., different target sites). In a set, each target site (e.g., HR1′, HR2′ and HR3′) is different, and the target sites are adjacent sites on the target nucleic acid, e.g., usually not more than 15 nucleotides distant, e.g., not more than at or about 10, 8, 6, 4, or 2 nucleotides away from the other site, and may be contiguous sites.

Target sites (e.g., hybridization regions in the target nucleic acid) are typically present on the same strand of the target nucleic acid in the same orientation. Target sites are also selected to provide a unique binding site, relative to other nucleic acids present in the cell. In some embodiments, the HR1′ and the HR2′ on the target nucleic acid are separated by 0 to 3 nucleotides, such as 0 to 2 nucleotides. In some embodiments, the HR1′ and the HR2′ are separated by 0 nucleotides (e.g., are contiguous). In some embodiments, the HR1′ and the HR2′ are separated by 1 nucleotide. In some embodiments, the HR1′ and the HR2′ are separated by 2 nucleotides. In some embodiments, the HR1′ and the HR2′ are separated by fewer than 2 nucleotides. In some embodiments, the HR2′ and the HR3′ on the target nucleic acid are separated by 0 to 3 nucleotides, such as 0 to 2 nucleotides. In some embodiments, the HR2′ and the HR3′ are separated by 0 nucleotides (e.g., are contiguous). In some embodiments, the HR2′ and the HR3′ are separated by 1 nucleotide. In some embodiments, the HR2′ and the HR3′ are separated by 2 nucleotides. In some embodiments, the HR2′ and the HR3′ are separated by fewer than 2 nucleotides.

In some embodiments, each of the target sites (HRs), such as HR1′, HR2′ or HR3′, are independently about 4 to about 40 nucleotides in length, such as about 4 to about 16 nucleotides in length or about 8 to about 12 nucleotides in length, for example, about 10 nucleotides. In some aspects, the length of one, two or all of the target site HR, such as HR1′, HR2′ or HR3′ is about 9 nucleotides. In some aspects, the length of one, two or all of the target site HR, such as HR1′, HR2′ or HR3′ is about 10 nucleotides. In some aspects, the length of one, two or all of the target site HR, such as HR1′, HR2′ or HR3′ is about 11 nucleotides. In some aspects, the length of one, two or all of the target site HR, such as HR1′, HR2′ or HR3′ is about 12 nucleotides. In some aspects, the length of one, two or all of the target site HR, such as HR1′, HR2′ or HR3′ is about 13 nucleotides. In some aspects, the length of one, two or all of the target site HR, such as HR1′, HR2′ or HR3′ is about 14 nucleotides. In some aspects, the length of one, two or all of the target site HR, such as HR1′, HR2′ or HR3′ is about 15 nucleotides. In some aspects, the length of one, two or all of the target site HR, such as HR1′, HR2′ or HR3′ is about 16 nucleotides. In some aspects, the length of one, two or all of the target site HR, such as HR1′, HR2′ or HR3′ is about 17 nucleotides. In some aspects, the length of one, two or all of the target site HR, such as HR1′, HR2′ or HR3′ is about 18 nucleotides. In some aspects, the length of one, two or all of the target site HR, such as HR1′, HR2′ or HR3′ is about 19 nucleotides. In some aspects, the length of one, two or all of the target site HR, such as HR1′, HR2′ or HR3′ is about 20 nucleotides. In some aspects, the length of one, two or all of the target site HR, such as HR1′, HR2′ or HR3′ is fewer than 9 nucleotides. In some aspects, the length of one, two or all of the target site HR, such as HR1′, HR2′ or HR3′ is fewer than 10 nucleotides. In some aspects, the length of one, two or all of the target site HR, such as HR1′, HR2′ or HR3′ is fewer than 11 nucleotides. In some aspects, the length of one, two or all of the target site HR, such as HR1′, HR2′ or HR3′ is fewer than 12 nucleotides. In some aspects, the length of one, two or all of the target site HR, such as HR1′, HR2′ or HR3′ is fewer than 13 nucleotides. In some aspects, the length of one, two or all of the target site HR, such as HR1′, HR2′ or HR3′ is fewer than 14 nucleotides. In some aspects, the length of one, two or all of the target site HR, such as HR1′, HR2′ or HR3′ is fewer than 15 nucleotides. In some aspects, the length of one, two or all of the target site HR, such as HR1′, HR2′ or HR3′ is fewer than 16 nucleotides. In some aspects, the length of one, two or all of the target site HR, such as HR1′, HR2′ or HR3′ is fewer than 17 nucleotides. In some aspects, the length of one, two or all of the target site HR, such as HR1′, HR2′ or HR3′ is fewer than 18 nucleotides. In some aspects, the length of one, two or all of the target site HR, such as HR1′, HR2′ or HR3′ is fewer than 19 nucleotides. In some aspects, the length of one, two or all of the target site HR, such as HR1′, HR2′ or HR3′ is fewer than 20 nucleotides. In some embodiments, each of the target sites (HRs), such as HR1′, HR2′ or HR3′, is independently about 8 to about 12 nucleotides in length. In some embodiments, each of the target sites (HRs), such as HR1′, HR2′ or HR3′, is independently fewer than about 8 to about 12 nucleotides in length.

In some embodiments, the hybridization regions (HRs) between the one or more polynucleotide probe sets and the target sites on the target nucleic acids comprises a length of polynucleotides that is sufficient for hybridization during the hybridization step and generally maintains the hybrid complex formation or does not substantially dissociate during subsequent steps of the methods or uses, for example, during the ligation, amplification and/or detection steps. In some aspects, the melting temperature (Tₘ) of the hybridization regions is selected to minimize occurrence of a ligation in solution. The “melting temperature” or “Tₘ” of a nucleic acid is defined as the temperature at which half of the helical structure of the nucleic acid is lost due to heating or other dissociation of the hydrogen bonding between base pairs, for example, by acid or alkali treatment. The Tₘ of a nucleic acid molecule depends on its length and on its base composition. Nucleic acid molecules rich in GC base pairs have a higher Tₘ than those having an abundance of AT base pairs. Separated complementary strands of nucleic acid spontaneously re-associate or anneal to form duplex nucleic acid when the temperature is lowered below the Tₘ. The highest rate of nucleic acid hybridization occurs approximately 25° C. below the Tₘ. The Tₘ may be estimated using the following relationship: Tₘ=69.3+0.41 (GC)% (Marmur et al. (1962) J. Mol. Biol. 5:109-118), but any suitable methodology for calculating Tₘ is contemplated herein.

In some embodiments, the set of polynucleotide probes, such as the first polynucleotide, the second polynucleotide and the third polynucleotides, are selected such that each polynucleotide in the set has a similar melting temperature (Tₘ) for binding to the corresponding target site/target hybridization regions (for example, HR1/HR1′, HR2/HR2′ or HR3/HR3′ as depicted in FIGS. 1 and 2). In some aspects, the Tₘ for the hybridization regions HR1/HR1′, HR2/HR2′ or HR3/HR3′ are the same or substantially the same. In some aspects, Tₘ that is “substantially the same” can refer to a Tₘ that exhibits similar degree of dissociation for different hybridization regions, and/or permits similar enzymatic reaction to occur at similar degrees in two different hybridization regions, and in some cases, is within at or about 5° C., 4° C., 3° C., 2° C., or 1° C. of the Tₘ for the other hybridization regions. In some embodiments, the Tₘ for HR1/HR1′ hybridization is within at or about 5° C., 4° C., 3° C., 2° C., or 1° C. of the Tₘ for HR2/HR2′ hybridization. In some embodiments, the Tₘ for HR1/HR1′ hybridization is within at or about 5° C., 4° C., 3° C., 2° C., or 1° C. of the Tₘ for HR3/HR3′ hybridization. In some embodiments, the Tₘ for HR2/HR2′ hybridization is within at or about 5° C., 4° C., 3° C., 2° C., or 1° C. of the Tₘ for HR3/HR3′ hybridization. In some embodiments, the Tₘ for HR2/HR2′ hybridization is within at or about 5° C., 4° C., 3° C., 2° C., or 1° C. of the Tₘ for HR1/HR1′ hybridization. In some embodiments, the Tₘ for HR3/HR3′ hybridization is within at or about 5° C., 4° C., 3° C., 2° C., or 1° C. of the Tₘ for HR1/HR1′ hybridization. In some embodiments, the Tₘ for HR3/HR3′ hybridization is within at or about 5° C., 4° C., 3° C., 2° C., or 1° C. of the Tₘ for HR2/HR2′ hybridization.

In some embodiments, the Tₘ of HR1/HR1′ hybridization, the Tₘ of HR2/HR2′ hybridization, and the Tₘ of HR3/HR3′ hybridization are substantially the same. In some embodiments, the Tₘ of HR1/HR1′ hybridization, the Tₘ of HR2/HR2′ hybridization, and/or the Tₘ of HR3/HR3′ hybridization are between about 40° C. and about 70° C. In some aspects, the Tₘ for one or each of HR1/HR1′, HR2/HR2′ and/or HR3/HR3′ hybridization is at least at or about or at or about 30° C., at or about 32° C., at or about 35° C., at or about 38° C., at or about 40° C., at or about 42° C., at or about 45° C., at or about 48° C., at or about 50° C., at or about 52° C., at or about 55° C., at or about 58°, at or about 60° C., at or about 62° C., at or about 65° C., at or about 68° C., at or about 70° C., at or about 72° C., at or about 75° C., at or about 78° C. or at or about 80° C., or within a range defined by any of the foregoing. In some aspects, the probe set of first polynucleotide, second polynucleotide and third polynucleotide are selected such that each target hybridization region in the set has a similar melting temperature (Tₘ) for binding to corresponding target site on the target nucleic acids, e.g., the Tₘ may be at least at or about or at or about 30° C., at or about 32° C., at or about 35° C., at or about 38° C., at or about 40° C., at or about 42° C., at or about 45° C., at or about 48° C., at or about 50° C., at or about 52° C., at or about 55° C., at or about 58°, at or about 60° C., at or about 62° C., at or about 65° C., at or about 68° C., at or about 70° C., at or about 72° C., at or about 75° C., at or about 78° C. or at or about 80° C., or within a range defined by any of the foregoing. In some embodiments, the GC content of each target hybridization regions are generally selected to be no more than at or about 20%, no more than at or about 30%, no more than at or about 40%, no more than at or about 50%, no more than at or about 60%, or no more than at or about 70%.

In some aspects, the provided embodiments involve contacting a target nucleic acid or a sample comprising the target nucleic acids with the one or more polynucleotide probe sets. In some aspects, the methods involve hybridizing the one or more polynucleotide probes with the target sites in the target nucleic acids. In some aspects, the hybridization permits pairing and hybridization of corresponding complementary pairs of hybridization regions of the one or more polynucleotide probes and the target sites in the target nucleic acids (for example, HR1 of the first polynucleotide and HR1′ of the target nucleic acid; HR2 of the second polynucleotide and HR2′ of the target nucleic acid; and HR3 of the third polynucleotide and HR3′ of the target nucleic acid) or the corresponding complementary pairs of hybridization regions of one of the polynucleotide probes and another polynucleotide probe (e.g., inter-polynucleotide probe hybridization regions, for example, HRa′ of the first polynucleotide and HRa of the second polynucleotide; or HRb1-HRb2 of the second polynucleotide and HRb′ of the third polynucleotide).

In some aspects, the melting temperature (Tₘ) of the HR between the probe polynucleotides and target sites on the target nucleic acid (e.g., HR1/HR1′, HR2/HR2′ or HR3/HR3′) is higher than the Tₘ of the inter-polynucleotide probe hybridization regions (e.g., HRa′ of the first polynucleotide and HRa of the second polynucleotide; or HRb1-HRb2 of the second polynucleotide and HRb′ of the third polynucleotide). The probe polynucleotide set can have a higher Tₘ for the hybridization between the HR in the probe polynucleotides (typically DNA) and the target nucleic acid (e.g., mRNA), for example, of at or about 40° C., 45° C., 50° C., 55° C., 60° C., 65° C. or 70° C. or higher, or any temperature in the range defined by the foregoing, and a relatively lower Tₘ for the inter-polynucleotide probe hybridization regions (typically between DNA molecules), for example, of lower than at or about 40° C., 35° C., 30° C., 25° C. or 20° C. or lower, or any temperature in the range defined by the foregoing. In some embodiments, the Tₘ of HRa/HRa′ hybridization and/or the Tₘ of HRb1-HRb2/HRb′ hybridization are lower than the Tₘ of HR1/HR1′ hybridization, the Tₘ of HR2/HR2′ hybridization, and/or the Tₘ of HR3/HR3′ hybridization. In some embodiments, the Tₘ of HRa/HRa′ hybridization and/or the Tₘ of HRb1-HRb2/HRb′ hybridization a are lower than about 40° C. or is similar to or lower than room temperature (in some examples, less than 30° C., or 22-25° C.). In some embodiments, the Tₘ of HRa/HRa′ hybridization and/or the Tₘ of HRb1-HRb2/HRb′ hybridization are lower than about 40° C.

### IV. HYBRIDIZATION, LIGATION, AMPLIFICATION, DETECTION AND SEQUENCING

In some aspects, the provided embodiments involve one or more various different steps for analyzing a target nucleic acid. In some aspects, the provided embodiments involve the use of one or more of the polynucleotides of the polynucleotide probe set, such as the three-polynucleotide set, as described herein, including performing one or more steps described herein. In some embodiments, the analyte to be analyzed using the polynucleotide probe set(s) is a target nucleic acid. In some aspects, the target nucleic acid is a nucleic acid present in a cell or a biological sample, such as a tissue sample. In some embodiments, the provided embodiments involve one or more steps from among hybridization, ligation, amplification and/or detection.

In some embodiments, the provided methods involve contacting a target nucleic acid with a first polynucleotide, a second polynucleotide, and a third polynucleotide to form a hybridization complex, for example, for the one or more polynucleotide(s) of the provided polynucleotide probe set to recognize and hybridize to its target on the target nucleic acid (e.g., mRNA).

In some embodiments, the provided methods involve circularizing (e.g., ligating the 5′ and 3′ ends of) a second polynucleotide (also called the “center probe” or the “padlock probe”) in a hybridization complex comprising a target nucleic acid, a first polynucleotide, the second polynucleotide, and a third polynucleotide. In some embodiments, the ligation circularizes the second polynucleotide.

In some embodiments, the provided methods involve forming an amplification product using a circularized second polynucleotide as a template and a first polynucleotide as a primer. In some aspects, the circularized second polynucleotide is amplified, for example, using rolling circle amplification (RCA). In some aspects, the amplifications step amplifies the sequences contained within the second polynucleotide, for example, including the one or more barcode sequence(s) within the second polynucleotide. In some aspects, the amplification step provides an amplified signal for detection and/or sequencing of the probe set polynucleotides and target nucleic acids.

In some aspects, the provided methods involve detecting a sequence in an amplification product, such as an amplified second polynucleotide. In some aspects, the detection involves determining the sequence of a portion of the amplification product, for example one or more barcode(s) of the second polynucleotide; and/or contacting with a detectable probe. In some aspects, the provided methods involve detecting a sequence of the first or third polynucleotide.

In some aspects, the provided methods involve a step of anchoring or linking the polynucleotide probe set, the target nucleic acid, hybridization complexes and amplification products to particular locations in the cell and/or the sample, such as to a matrix, other nucleic acids present in the sample and/or to cellular structures. In some embodiments, the one or more steps selected from among hybridization, ligation, amplification and/or detection takes place at the location of or associated with the target nucleic acid in the sample.

In some embodiments, the provided methods involve one or more of the steps described herein. In some embodiments, a plurality of the steps described herein are performed, sequentially or simultaneously. In some embodiments, one of the steps are performed on a sample that has been previously processed, for example, using one of the steps described herein or similar known methods or processes. In some embodiments, one or more steps are performed on a sample that has been previously processed. In some of embodiments, the hybridization, ligation and/or amplification steps can be performed sequentially or simultaneously. In some aspects, the hybridization step is performed before ligation. In some aspects, the amplification step is performed after ligation. In some embodiments, the hybridization step is performed after ligation. In some aspects, one or more steps of the hybridization, ligation and/or amplification is performed simultaneously. In some aspects, the methods comprise the steps of hybridization, ligation and amplification, sequentially performed in that order, followed by detection and/or sequencing.

In some embodiments, the provided methods involve one or more steps of: contacting a target nucleic acid with a first polynucleotide, a second polynucleotide, and a third polynucleotide to form a hybridization complex; circularizing the second polynucleotide; forming an amplification product using the circularized second polynucleotide as a template and the first polynucleotide as a primer; and/or detecting the amplification product, wherein the detection is indicative of the target nucleic acid or a sequence thereof. In some embodiments, the provided methods involve contacting a target nucleic acid with a first polynucleotide, a second polynucleotide, and a third polynucleotide to form a hybridization complex; circularizing the second polynucleotide; forming an amplification product using the circularized second polynucleotide as a template and the first polynucleotide as a primer; and detecting the amplification product, wherein the detection is indicative of the target nucleic acid or a sequence thereof, performed in such order.

In some aspects, the provided embodiments include a method that involves one or more of the following: hybridization of the polynucleotide probes; specific signal amplification, for example by rolling-circle amplification; detection of one or more portions of a polynucleotide or amplified nucleic acids; an image-based in situ nucleic acid (DNA and/or RNA) sequencing, for example, by sequencing-by-ligation; anchoring one or more portions of the amplified nucleic acids to a scaffold or a matrix (see, for example, Section V herein); and associated data analysis.

In some embodiments, the hybridization complex is formed at a temperature between about 30° C. and about 50° C., optionally about 40° C.; the circularization of the second polynucleotide comprises a ligation reaction, and the ligation reaction is performed at a temperature between about 10° C. and about 30° C., optionally between about 15° C. and about 25° C.; the amplification is performed at a temperature between about 15° C. and about 35° C., optionally about 30° C.; and/or the detection comprises determining the sequence of all or a portion of the amplification product.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target nucleic acid sequence for the probe set described herein, may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe, for example another polynucleotide probe of the probe set. The exogenously added nucleic acid probe may be optionally ligated to a cellular nucleic acid molecule or another exogenous nucleic acid molecule. In other examples, a product comprising a target nucleic acid sequence for a probe disclosed herein may be a rolling circle amplification product of a circularizable probe or probe set which hybridizes to a cellular nucleic acid molecule (e.g., genomic DNA or mRNA) or product thereof (e.g., a transcript such as cDNA). In other examples, a product comprising a target sequence for a probe disclosed herein (e.g., polynucleotide probe set) may a probe hybridizing to an RCA product. The probe may comprise an overhang that does not hybridize to the RCA product but hybridizes to another probe (e.g., first or third polynucleotide of the polynucleotide probe set). The probe may be optionally ligated to a cellular nucleic acid molecule or another probe, e.g., an anchor probe that hybridize to the RCA product.

A. Hybridization

In some aspects, the target nucleic acid or a biological sample containing the target nucleic acid, such as a cell or a tissue sample, is contacted with one or more polynucleotide from the polynucleotide probe set. In some embodiments, the provided methods involve contacting a target nucleic acid with a first polynucleotide, a second polynucleotide, and a third polynucleotide to form a hybridization complex. In some aspects, the one or more polynucleotide(s) is contacted with the target nucleic acid or a biological sample containing the target nucleic acid under a condition that permits formation of the hybridization complex between the one or more polynucleotide probes to the corresponding target sites of the target nucleic acid. In some aspects, the contacting of the target nucleic acid with the polynucleotide probe set can be referred to as the “hybridization” step.

In some aspects, the terms “hybridize” and “hybridization” can refer to the formation of complexes between nucleotide sequences which are sufficiently complementary to form complexes via Watson-Crick base pairing. Where a primer sequence “hybridizes” with target (template), such complexes (or hybrids) are sufficiently stable to serve the priming function required by, e.g., the DNA polymerase to initiate DNA synthesis. It will be appreciated that the hybridizing sequences need not have perfect complementarity to provide stable hybrids. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. In many situations, stable hybrids will form where fewer than about 10% of the bases are mismatches, ignoring loops of four or more nucleotides. In some aspects, the term “complementary” refers to an polynucleotide that forms a stable duplex with its “complement” under certain assay conditions. In some aspects, the term “near-complementary” refers to an polynucleotide that forms a stable duplex with its “complement” under certain assay conditions, but has less than 100% complementary sequences, such as about 90% or greater complementary sequences.

In some aspects, the polynucleotide probes are denatured prior to hybridization, for example by heating to a temperature of at least about 50° C., at least about 60° C., at least about 70° C., at least about 80° C., and up to about 99° C., up to about 95° C., or up to about 90° C.

In some embodiments, the hybridization between the hybridization regions on the probe polynucleotides and the target sites on the target nucleic acid is performed under conditions to allow for specific hybridization between each of the corresponding target site/target hybridization regions. For example, the target nucleic acid contains target sites HR1′, HR2′, and HR3′, which can hybridize to target hybridization regions HR1 of the first polynucleotide, HR2 of the second polynucleotide, and HR3 of the third polynucleotide, respectively (with reference to the HRs depicted in the exemplary schematic set forth in FIGS. 1 and 2). Following hybridization, the first polynucleotide (e.g., left probe), the second polynucleotide (e.g., center probe) and the third polynucleotide (e.g., right probe) will hybridize in a sequential configuration, in the 3′ to 5′ direction of the target nucleic acid.

In some instances, the hybridization between the HR in the probe polynucleotides and the target nucleic acid (e.g., for hybridization of HR1/HR1′, HR2/HR2′ or HR3/HR3′ as depicted in FIGS. 1 and 2) is performed at a temperature that is lower than the Tₘ for the hybridization of HR1/HR1′, HR2/HR2′ and/or HR3/HR3′, but at a temperature that is higher than the Tₘ of the inter-polynucleotide probe hybridization regions (e.g., HRa′ of the first polynucleotide and HRa of the second polynucleotide; or HRb1-HRb2 of the second polynucleotide and HRb′ of the third polynucleotide). In some aspects, such differences in Tₘ permits the hybridization between the probe polynucleotides and target sites on the target nucleic acid while reducing or minimizing the inter-polynucleotide probe hybridization during the hybridization step. In some embodiments, the subsequent steps, such as ligation, amplification and/or detection can be performed at a temperature that is lower than the Tₘ of the HRs between the probe polynucleotides and target site on the target nucleic acid, at a temperature required for the reaction at the particular step. In some aspects, the hybridization occurs at a temperature of less than the lowest Tₘ among the Tₘ for HR1/HR1′, HR2/HR2′ and/or HR3/HR3′ hybridization. In some aspects, the hybridization between the HR in the probe polynucleotides and the target nucleic acid can be maintained during subsequent steps of the methods such as the ligation, amplification or detection steps. In some embodiments, the hybridization complex is formed at a temperature higher than Tm of HRa/HRa′ hybridization and/or the Tₘ of HRb1/HRb2/HRb′ hybridization, but lower than the Tₘ of HR1/HR1′ hybridization, the Tₘ of HR2/HR2′ hybridization, and/or the Tₘ of HR3/HR3′ hybridization.

In some embodiments, the Tₘ for one or each of HR1/HR1′, HR2/HR2′ and/or HR3/HR3′ hybridization is at least at or about or at or about 30° C., at or about 32° C., at or about 35° C., at or about 38° C., at or about 40° C., at or about 42° C., at or about 45° C., at or about 48° C., at or about 50° C., at or about 52° C., at or about 55° C., at or about 58°, at or about 60° C., at or about 62° C., at or about 65° C., at or about 68° C., at or about 70° C., at or about 72° C., at or about 75° C., at or about 78° C. or at or about 80° C., or within a range defined by any of the foregoing. In some aspects, the hybridization complex between one or each of HR1/HR1′, HR2/HR2′ and/or HR3/HR3′ is formed at a temperature that is at or about or less than at or about 30° C., at or about 32° C., at or about 35° C., at or about 38° C., at or about 40° C., at or about 42° C., at or about 45° C., at or about 48° C., at or about 50° C., at or about 52° C., at or about 55° C., at or about 58°, at or about 60° C., at or about 62° C., at or about 65° C., at or about 68° C., at or about 70° C., at or about 72° C., at or about 75° C., at or about 78° C. or at or about 80° C., or within a range defined by any of the foregoing. In some embodiments, the hybridization complex is formed at a temperature between about 30° C. and about 50° C. In some embodiments, the hybridization complex is formed at about 40° C. In some embodiments, the hybridization complex is formed at a temperature between about 40° C. and about 80° C. In some embodiments, the hybridization complex is formed at a temperature between about 50° C. and about 70° C. In some embodiments, the hybridization complex is formed at about 60° C.

In some aspects, selective hybridization occurs when two nucleic acid sequences are substantially complementary, i.e., at least about 65% 75%, 80%, 85%, 90%, 91%, 92%, 93%, 94%, 95%, 96%, 97%, 98% 99%, 99.1%, 99.2%, 99.3%, 99.4%, 99.5%, 99.6%, 99.7%, 99.8%, 99.9% or 100% complementary over a stretch of at least 14 to 25 nucleotides. See Kanehisa, M., 1984, Nucleic Acids Res. 12: 203, incorporated herein by reference in its entirety for all purposes. Exemplary factors influence the efficiency and selectivity of hybridization of nucleic acids include: (i) primer length, (ii) the nucleotide sequence and/or composition, (iii) hybridization temperature, (iv) buffer chemistry and (v) the potential for steric hindrance in the region to which the nucleic acid is required to hybridize. Such factors can influence the design of the hybridization regions and/or the conditions for hybridization. In some aspects, there is a positive correlation between nucleic acid length and both the efficiency and accuracy with which a nucleic acid will anneal to a target sequence; longer sequences have a higher Tₘ than do shorter ones, and are less likely to be repeated within a given target sequence, thereby reducing promiscuous hybridization.

Nucleic acid sequences with a high G-C content or that comprise palindromic sequences tend to self-hybridize, as do their intended target sites, since unimolecular, rather than bimolecular, hybridization kinetics are generally favored in solution; at the same time, it is important to design a hybridization region containing sufficient numbers of G-C nucleotide pairings to bind the target sequence tightly, since each such pair is bound by three hydrogen bonds, rather than the two that are found when A and T bases pair. Hybridization temperature varies inversely with nucleic acid annealing efficiency, as does the concentration of organic solvents, e.g., formamide, that might be included in a hybridization mixture, while increases in salt concentration facilitate binding. In some aspects, under stringent hybridization conditions, longer probes hybridize more efficiently than do shorter ones, which are sufficient under more permissive conditions. Stringent hybridization conditions typically include salt concentrations of less than about 1M, more usually less than about 500 mM and preferably less than about 200 mM. Longer fragments may require higher hybridization temperatures for specific hybridization. As several factors affect the stringency of hybridization, the combination of parameters is more important than the absolute measure of any one alone. Hybridization conditions are known and include those described in Current Protocols in Molecular Biology, John Wiley & Sons, N.Y. (1989), 6.3.1-6.3.6. Examples of evaluation of parameters and the optimization of primer sequences are described in, for example, Hoover et al. (2002) Nucleic Acids Res. 30:e43, and Rouillard et al. (2004) Nucleic Acids Res. 32:W176, incorporated by reference herein in their entirety for all purposes.

B. Ligation

In some embodiments, the methods involve ligating the 5′ and 3′ ends of one of the polynucleotide probes, for example, to circularize a linear polynucleotide. In some embodiments, the provided methods involve circularizing a second polynucleotide (also called the “center probe” or the “padlock probe”) in a hybridization complex comprising a target nucleic acid, a first polynucleotide, the second polynucleotide, and a third polynucleotide. In some embodiments, the 5′ and 3′ ends of the second polynucleotide is ligated. In some aspects, a sample containing the one or more probe polynucleotides is incubated with a ligase under a condition that permits the ligation between the 5′ end and 3′ end of the second polynucleotide. In some embodiments, the circularization or the ligation of the second polynucleotide is performed after contacting the target nucleic acid or a biological sample containing the target nucleic acid with the one or more polynucleotide(s) of the polynucleotide probe set.

In some aspects, the third polynucleotide (e.g., right probe) contains hybridization region sequences (for example, HRb′ with reference to the schematic depicted in FIGS. 1 and 2) that can act as a splint to align (e.g., juxtapose) the 5′ and 3′ ends of the second polynucleotide (for example, comprising HRb1 and HRb2 with reference to the schematic depicted in FIGS. 1 and 2) for ligation and circularization. In some aspects, such circularized polynucleotide becomes a template for rolling circle amplification (RCA), and sequences contained in the second polynucleotide can be amplified after ligation. In some embodiments, the methods involve ligating the HRb1 and the HRb2 using HRb′ as a template, without gap filling. In some embodiments, the methods involve gap-filling and ligating HRb1 and HRb2 using HRb′ as a template.

In some aspects, a “splint” or a “splint template” is nucleotide sequence that, when hybridized to other nucleotide sequences, acts as a “splint” to position the polynucleotides next to one another so that they can be ligated together. In some embodiments, the splint is DNA or RNA. The splint can include a nucleotide sequence that is partially complimentary to nucleotide sequences from two or more different templates. In some embodiments, the splint assists in ligating a 5′ end and a 3′ end of a linear polynucleotide, resulting in a circular polynucleotide. In general, an RNA ligase, a DNA ligase, or another other variety of ligase is used to ligate two nucleotide sequences together.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. In some embodiments, a ligase is employed to ligate the second polynucleotide (e.g., central probe). In some aspects, the ligation of the 5′ and 3′ ends of the second polynucleotide results in a generation of a closed circular nucleic acid (i.e., circularizes a linear polynucleotide). In some aspects, the ligase is a DNA ligase. The term “ligase” as used herein includes an enzyme that is commonly used to join polynucleotides together or to join the ends of a single polynucleotide. Ligases include ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases include bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. Other exemplary ligases include those described in, for example, U.S. Ser. No. 10/626,390, US 2005/0266487, US 2010/0021941, US 2013/0143276, US 2014/0106431, US 2015/0210998, US 2015/0322478, US 2016/0201049, US 2017/0226498, U.S. Pat. Nos. 5,652,107, 5,700,672, 6,153,384, 6,444,429, 6,576,453, 6,737,244, 6,740,495, 6,787,341, 6,949,370, 7,781,182, 7,892,772, 7,927,853, 7,981,653, 8,137,943, 8,541,218, 8,546,094, 8,790,873, 9,127,269, 9,273,301, 9,284,541, 9,719,081, 9,725,708, U.S. Ser. No. 10/626,390, US 20090142811, US 20110143958, US 20150175998, US 20190062827, U.S. Pat. Nos. 6,280,998, 9,719,081 and 9,796,997. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. In some embodiments, the 5′ and 3′ ends of the second polynucleotide may be ligated directly or indirectly. “Direct ligation” of the ends of the second polynucleotide means that the ends of the polynucleotides (e.g., comprising HRb1 and HRb2 at the 5′ and 3′ ends, respectively) hybridize immediately adjacently on the splint (e.g., HRb′ on the third polynucleotide) to form a substrate for a ligase enzyme resulting in their ligation to each other (intramolecular ligation). Alternatively, “indirect” means that the ends of the polynucleotides (e.g., comprising HRb1 and HRb2 at the 5′ and 3′ ends, respectively) hybridize non-adjacently on the splint (e.g., HRb′ on the third polynucleotide), i.e., separated by one or more intervening nucleotides or “gaps”. In some embodiments, said ends are not ligated directly to each other, but circularization of the probe instead occurs either via the intermediacy of one or more intervening (so-called “gap” or “gap-filling” (oligo)nucleotides) or by the extension of the 3′ end of the probe to “fill” the “gap” corresponding to said intervening nucleotides (intermolecular ligation). In some cases, the gap of one or more nucleotides between the hybridized ends of the second polynucleotide may be “filled” by one or more “gap” (oligo)nucleotide(s) which are complementary to the splint on the third polynucleotide, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of the second polynucleotide, e.g., a gap oligonucleotide. In some cases, circularization of the second polynucleotide involves ligation of the ends of the probe to at least one gap (oligo)nucleotide, such that the gap (oligo)nucleotide becomes incorporated into the resulting circularized second polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

C. Amplification

In some embodiments, the methods involve a step of amplifying one or more probe polynucleotides. In some aspects, the amplification step includes amplifying the second polynucleotide (e.g., central probe, also called a padlock probe), for example, by rolling circle amplification (RCA). In some embodiments, the provided methods involve forming an amplification product using a circularized second polynucleotide as a template and a first polynucleotide as a primer. In some aspects, the circularized second polynucleotide is contacted with a polymerase for amplification under a condition that permits polymerization, for example, by RCA, of the second polynucleotide. In some embodiments, the amplification of the second polynucleotide is performed after circularization or the ligation of the second polynucleotide and/or contacting the target nucleic acid or a biological sample containing the target nucleic acid with the one or more polynucleotide(s) of the polynucleotide probe set.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing RCA. In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the RCA is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, the second polynucleotide is amplified after ligation of the 5′ and 3′ ends, to generate a closed nucleic acid circle. In some aspects, the amplification step is or includes an isothermal enzymatic amplification, such as an RCA. In some embodiments, the amplification step results in amplification of one or more of the probe polynucleotides, such as a polynucleotide comprising a barcode sequence. In some aspects, the amplification step generates a complex comprising the amplification product (also called “amplicon” or “amplified product”) and/or one or more probe polynucleotides. In some aspects, such complexes include a nanometer scale nucleic acid ball (also called “nanoball”). In some embodiments, after the formation of the hybridization complex and association of the amplification probe, the hybridization complex is rolling-circle amplified to generate a cDNA nanoball (i.e., amplicon) containing multiple copies of the cDNA.

In some embodiments, the provided methods include the step of performing RCA in the presence of a primer. In some embodiments, the primer provides the 3′ hydroxyl group (3′-OH) that are required by DNA polymerases. In some aspects, DNA polymerases add nucleotides to the 3′ end of a polynucleotide chain. In some aspects, the polymerase catalyzes the nucleophilic attack of the 3′-hydroxyl group terminus of the polynucleotide chain on the α-phosphate group of the nucleoside triphosphate to be added. In some embodiments, the 3′-OH for the primer to initiate amplification is provided by the first polynucleotide (e.g., left probe). In some embodiments, the circularized second polynucleotide is subject to RCA, primed by the 3′ ends of the first polynucleotide. In some aspects, the first polynucleotide comprises one or more nucleotides in the hybridization region (HR) that is complementary to or sufficiently complementary to one or more regions of the second polynucleotide (for example, HRa as depicted in FIGS. 1 and 2), and the at least one nucleotide from the 3′ end of the first polynucleotide acts as a primer, e.g., providing the 3′-OH for polymerization for RCA. In some embodiments, the amplification, e.g., RCA, includes using the second polynucleotide, such as the ligated or circularized second polynucleotide, as a template and the 3′ end of the first polynucleotide as a primer for a polymerase to form one or more amplicons. In some embodiments, a single-stranded, circular polynucleotide template is formed by ligation of the second nucleotide.

The second polynucleotide includes a hybridization region (HR; for example, HRa as depicted in FIGS. 1 and 2) that is complementary to a region in the first polynucleotide, for example, HRa′ as depicted in FIGS. 1 and 2). In some aspects, upon addition of a DNA polymerase in the presence of appropriate dNTP precursors and other cofactors, the second polynucleotide is amplified by binding of the primer (e.g., from the first polynucleotide) and elongation by addition of nucleotides, to result in multiple copies of the second polynucleotide as a the template. In some aspects, the amplification product is a long polynucleotide amplification product (i.e., amplicon). In some embodiments, only when a first polynucleotide, a second polynucleotide and a third polynucleotide hybridize to the same target nucleic acid, the second polynucleotide can be circularized and rolling-circle amplified to generate the a nanometer scale nucleic acid ball (also called “DNA nanoball” or “nanoball”). The DNA nanoball contains multiple copies of the DNA. The term “amplicon” refers to the amplified nucleic acid product of the RCA reaction or other nucleic acid amplification process.

In some embodiments, rolling circle amplification (RCA) is used for amplification. Rolling circle amplification or “RCA” of the circularized second polynucleotide results in the synthesis of a concatemeric amplification product containing numerous tandem repeats of the second polynucleotide. Any suitable known RCA reactions and conditions can be employed as appropriate. The ligation reaction may be carried out at the same time (i.e., simultaneously) as the RCA reaction of step, i.e., in the same step. In some embodiments, the RCA reaction is primed by the 3′ end of the first polynucleotide (e.g., HRa′) to which the second polynucleotide (e.g., HRa) has hybridized. In certain aspects, the primer (e.g., HRa′ region of the first polynucleotide) hybridizes to a region of the second polynucleotide other than the 5′ and 3′ terminal regions of the second polynucleotide.

Exemplary methods for RCA include those described in, for example, Baner et al., Nucleic Acids Research, 26:5073-5078, 1998; Lizardi et al., Nature Genetics 19:226, 1998; Mohsen et al., Acc Chem Res. 2016 Nov. 15; 49(11): 2540-2550; Schweitzer et al. Proc. Natl Acad. Sci. USA 97:10113-10119, 2000; Faruqi et al., BMC Genomics 2:4, 2000; Nallur et al., Nucl. Acids Res. 29:e118, 2001; Dean et al. Genome Res. 1 1:1095-1099, 2001; Schweitzer et al., Nature Biotech. 20:359-365, 2002; U.S. Pat. Nos. 6,054,274, 6,291,187, 6,323,009, 6,344,329 and 6,368,801). Exemplary polymerases for use in RCA include DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In the RCA reaction, the polymerase extends the 3′ end of the first polynucleotide (e.g., comprising the HRa′, serving as the primer) using a circularized polynucleotide, such as the circularized second polynucleotide as template. As a result of RCA, concatemeric amplification products containing numerous tandem repeats of the second polynucleotide are produced and may be detected as indicative of the presence and/or nature of a target nucleic acid (e.g., mRNA) in the sample.

Other methods of amplification can also be used, for example, the use of PCR, such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA. 91:360-364; incorporated herein by reference in their entirety for all purposes). Alternative amplification methods include: self-sustained sequence replication (Guatelli et al. (1990) Proc. Natl. Acad. Sci. USA 87:1874), transcriptional amplification system (Kwoh et al. (1989) Proc. Natl. Acad. Sci. US. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790) or any other nucleic acid amplification method using well known techniques. A variety of amplification methods are described in, for example, U.S. Pat. Nos. 6,391,544, 6,365,375, 6,294,323, 6,261,797, 6,124,090 and 5,612,199.

In some aspects, the amplification steps can be performed at a temperature that is lower than the Tₘ of hybridization of the HRs between the probe polynucleotides and target site on the target nucleic acid, at a temperature required for the amplification step. In some aspects, the binding of the primer and the amplification occurs in a temperature that is similar to or lower than the Tₘ of hybridization of the HRa′ of the first polynucleotide and the complementary HRa of the second polynucleotide. In some aspects, the amplification step, such as the rolling circle amplification (RCA) is performed at a temperature between at or about 22° C. and at or about 35° C., such as at or about 22° C., 23° C., 24° C., 25° C., 26° C., 27° C., 28° C., 29° C., 30° C., 31° C., 32° C., 33° C., 34° C., or 35° C.

In some aspects, during the amplification step, modified nucleotides can be added to the reaction to incorporate the modified nucleotides in the amplification product (e.g., nanoball). Exemplary of the modified nucleotides include amine-modified nucleotides. In some aspects, the modified nucleotides can be employed. In some aspects of the methods, for example, for anchoring or cross-linking of the generated amplification product (e.g., nanoball) to a scaffold, to cellular structures and/or to other amplification products (e.g., other nanoballs). In some aspects, the amplification products includes a modified nucleotide, such as an amine-modified nucleotide. In some embodiments, the amine-modified nucleotide includes an acrylic acid N-hydroxysuccinimide moiety modification. Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the methods involve amplifying nucleic acids in situ within the matrix by contacting the nucleic acids within the matrix with reagents and under suitable reaction conditions sufficient to amplify the nucleic acids. In some aspects, exemplary matrix includes those described herein, for example, in Section V.D. In some aspects, the matrix is porous to allow migration of reagents into the matrix to contact the nucleic acids. In some aspects, the polynucleotide, such as the second polynucleotide, is amplified by selectively hybridizing an amplification primer (e.g., at the 3′ end of the first polynucleotide) to an amplification site. Amplification primers may be present in solution to be added to the matrix or they may be added during formation of the matrix to be present therein sufficiently adjacent to nucleic acids to allow for hybridization and amplification.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a rolling circle amplification product from a circular RCA template which is provided or generated in the assay, and the RCA product is detected to detect the analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (i.e. a reagent in a detection assay), which is used as a reporter for the assay, or a part of a reporter, or signal-generation system. The RCA template used to generate the RCA product may thus be a circular (e.g. circularized) reporter nucleic acid molecule, namely from any RCA-based detection assay which uses or generates a circular nucleic acid molecule as a reporter for the assay.

D. Analysis of Sequences

In some aspects, the provided methods involve analyzing a sequence present in the polynucleotide probe set and/or in an amplification product, such as an amplified second polynucleotide. In some embodiments, the analyzing comprises determining the sequence of all or a portion of the amplification product. In some embodiments, the analyzing comprises detecting a sequence present in the amplification product. In some aspects, the analysis involves determining the sequence of a portion of the amplification product. In some aspects, the analysis involves contacting the target nucleic acid, amplification product or biological sample containing the target nucleic acid or amplification product, with a detectable probe. In some embodiments, the provided methods involve determining a sequence present in the polynucleotide probe set and/or in an amplification product, wherein the sequence is indicative of the presence and/or the amount of a target nucleic acid or a sequence thereof. In some aspects, the amplification product is formed using a circularized second polynucleotide as a template and a first polynucleotide as a primer. In some aspects, the amplification product (e.g., circularized second polynucleotide) includes a barcode sequence, which is also amplified during the amplification step. In some aspects, an amplified sequence, such as the amplified barcode sequence present in the second polynucleotide can be analyzed and/or detected. In some aspects, the sequence of the barcode is determined. In some aspects, the analysis of the barcode sequence involves sequencing. In some embodiments, detection of the barcodes includes detecting an amplified signal associated with the barcode sequences.

In some embodiments, the analysis and/or determination of sequence of the second polynucleotide is performed after amplification of the second polynucleotide, circularization or the ligation of the second polynucleotide and/or contacting the target nucleic acid or a biological sample containing the target nucleic acid with the one or more polynucleotide(s) of the polynucleotide probe set. In some aspects, the analysis and/or sequence determination also include one or more of: obtaining images or detection of signal from the detectable probe and/or data analysis.

In some aspects, the analysis of the sequence comprises determining the sequence of all or a portion of the amplification product. In some embodiments, the determining the sequence comprises sequencing all or a portion of the amplification product, such as one or more barcode sequence(s) present in the amplified product. In some embodiments, the sequencing can include sequencing by hybridization, sequencing by ligation, and/or fluorescent in situ sequencing.

In some aspects, the analysis of the sequence comprises in situ hybridization to all or a portion of the amplification product. In some aspects, analyzing the sequence comprises hybridizing to the amplification product a detection oligonucleotide labeled with a fluorophore, an isotope, a mass tag, or a combination thereof. In some embodiments, the detecting comprises in situ hybridization to one or more barcode sequence(s). In some embodiments, the in situ hybridization comprises sequential fluorescent in situ hybridization.

In some embodiments, the analyzing the sequence comprises imaging one or more amplification products.

In some embodiments, the target nucleic acid is an mRNA in a tissue sample, and the analyzing the sequence is performed when the target nucleic acid and/or the amplification product is in situ in the tissue sample.

In some aspects, the amplification product (e.g., amplicon) and/or one or more portions of the probe set (for example, a portion of the first, the second or the third polynucleotide) is detected. In some embodiments, the provided methods include a step for analysis of a sequence, by detection of all or a portion of the amplification product. In some aspects, the amplification product can be detected via a detection probe that comprises a detectable label and is able to recognize one or more portions of the amplification product and/or one or more portions of the probe set, such as the barcode region. In some aspects, the detection probe comprises a detection oligonucleotide. In some aspects, the detection probe comprises a detectable label, for example, a fluorophore, an isotope, a mass tag, or a combination thereof.

In some embodiments, the analysis and/or sequence determination comprises sequencing all or a portion of the amplification product and/or in situ hybridization to the amplification product. In some embodiments, the sequencing step involves sequencing hybridization, sequencing by ligation, and/or fluorescent in situ sequencing, and/or wherein the in situ hybridization comprises sequential fluorescent in situ hybridization. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some embodiments, the sequence determination or analysis comprises sequencing all or a portion of any of the polynucleotides of the probe set, such as one or more barcode sequence(s) present in the first, second, and/or third polynucleotide. In some embodiments, determining the sequence may include any suitable methods described herein. In some embodiments, detection of the barcodes includes detecting an amplified signal associated with the barcode sequences in situ. In some examples, the amplification can be performed using a non-enzymatic amplification method. In some embodiments, the amplification of the signal refers to an amplified signal associated with the barcode, which may include hybridization chain reaction (HCR) directly or indirectly on the one or more barcode sequences, linear oligonucleotide hybridization chain reaction (LO-HCR) directly or indirectly on the one or more barcode sequences, primer exchange reaction (PER) directly or indirectly on the one or more barcode sequences, assembly of branched structures directly or indirectly on the one or more barcode sequences, hybridization of a plurality of detectable probes directly or indirectly on the one or more barcode sequences, or any combination thereof. In some embodiments, a method disclosed herein may also comprise one or more signal amplification components. In some embodiments, detection of an amplified signal associated with the barcode sequences in situ is used to detect a barcode of the first and/or third polynucleotide which has not been amplified using RCA.

Exemplary signal amplification methods include targeted deposition of detectable reactive molecules around the site of probe hybridization, targeted assembly of branched structures (e.g., bDNA or branched assay using locked nucleic acid (LNA)), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER).

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, WO 2019/236841, WO 2020/102094, WO 2020/163397, and WO 2021/067475, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 incorporated herein by reference), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule.

In some embodiments, detection of nucleic acids sequences (e.g. barcodes) in situ includes an assembly for branched signal amplification. In some embodiments, the assembly complex comprises an amplifier hybridized directly or indirectly (via one or more oligonucleotides) to a sequence of the first, second, and/or third polynucleotide. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, detection of nucleic acids sequences (e.g. barcodes) in situ comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising the first polynucleotide, the second polynucleotide and the third polynucleotides. In various embodiments, the first polynucleotide, the second polynucleotide and the third polynucleotides may be contacted with a plurality of concatemer primers and a plurality of labeled probes. see e.g., U.S. Pat. Pub. No. US20190106733, which is incorporated herein by reference, for exemplary molecules and PER reaction components.

In some aspects, the provided methods include imaging the amplification product (e.g., amplicon) and/or one or more portions of the probe set, for example, via binding of the detection probe and detecting the detectable label. In some embodiments, the detection probe includes a detectable label that can be measured and quantitated. The terms “label” and “detectable label” include a directly or indirectly detectable moiety that is associated with (e.g., conjugated to) a molecule to be detected, e.g., a detectable probe, including, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorophore” includes a substance or a portion thereof that is capable of exhibiting fluorescence in the detectable range. Particular examples of labels that may be used in accordance with the provided embodiments include, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

In some embodiments, a detectable probe containing a detectable label can be used to detect one or more polynucleotide(s) and/or amplification products (e.g., amplicon) described herein. In some embodiments, the methods involve incubating the detectable probe containing the detectable label with the sample, washing unbound detectable probe, and detecting the label, e.g., by imaging.

Examples of detectable labels include but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers include, but are not limited to, luciferase (e.g., bacterial, firefly and click beetle), luciferin, aequorin and the like. Examples of enzyme systems having visually detectable signals include, but are not limited to, galactosidases, glucorimidases, phosphatases, peroxidases and cholinesterases. Identifiable markers also include radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels include those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments include those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” includes a signaling moiety that conveys information through the fluorescent absorption and/or emission properties of one or more molecules. Exemplary fluorescent properties include fluorescence intensity, fluorescence lifetime, emission spectrum characteristics and energy transfer.

Examples of commercially available fluorescent nucleotide analogues readily incorporated into nucleotide and/or polynucleotide sequences include, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment include, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, including, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into an polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for an polynucleotide sequence may include fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens include, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting includes performing microscopy, scanning mass spectrometry or other imaging techniques described herein. In such aspects, the detecting includes determining a signal, e.g., a fluorescent signal.

In some aspects, the detection (including imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” includes any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed include bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some aspects, known methods and techniques for detecting, visualizing and analyzing particular sequence, such as a particular barcode sequence, can be used to detect and identify a portion of the polynucleotide probe set and/or amplification products, for example the one or more barcode(s) present in the amplified second polynucleotide. In some aspects, nucleic acid barcoding can be used to detect and/or identify a target nucleic acid (e.g., an mRNA) among various nucleic acids present in a sample. In some aspects, by virtue of the hybridization between the target sites of the target nucleic acid and the corresponding target hybridization regions of the polynucleotide probe set, particular target nucleic acid can be identified. In addition, as one of the polynucleotides in the polynucleotide probe set (e.g., second polynucleotide) is amplified, particular sequences (e.g., barcodes) present in the amplification product can be detected even at low levels, by virtue of the amplification of the number of copies of the barcodes present. In some embodiments, exemplary methods of nucleic acid barcoding and detection or identification include those described herein and/or any suitable methods.

In some embodiments, the one or more barcode sequences in the amplicon are contacted with detection probes configured to detect (e.g., through hybridization of labeled probes) the barcode sequence. In some embodiments, the detection probes are two or more nucleotides in length. In some embodiments, the detection probes are at or about two nucleotides in length. In some embodiments, the detection probes are at or about three nucleotides in length. In some embodiments, the detection probes are at or about four nucleotides in length. In some embodiments, the detection probes are at or about five nucleotides in length. In some embodiments, the detection probes are at or about five nucleotides in length. In some embodiments, the detection probes are at or about six nucleotides in length. In some embodiments, the detection probes are at or about seven nucleotides in length. In some embodiments, the detection probes are at or about eight nucleotides in length. In some embodiments, the detection probes are at or about nine nucleotides in length. In some embodiments, the detection probes are at or about 10 nucleotides in length. In some embodiments, the detection probes are at or about 12 nucleotides in length. In some embodiments, the detection probes are at or about 14 nucleotides in length. In some embodiments, the detection probes are at or about 16 nucleotides in length. In some embodiments, the detection probes are at or about 18 nucleotides in length. In some embodiments, the detection probes are at or about 20 nucleotides in length. In some embodiments, the detection probes are at or about 22 nucleotides in length. In some embodiments, the detection probes are at or about 24 nucleotides in length. In some embodiments, the detection probes are at or about 26 nucleotides in length. In some embodiments, the detection probes are at or about 28 nucleotides in length. In some embodiments, the detection probes are at or about 30 nucleotides in length. In some embodiments, the use of an 8-nt barcode would enable sequencing of the entire transcriptome. In some embodiments, the use of an 8-nt barcode would enable sequencing of 65,536 genes.

The detection probes can be labeled. In some embodiments, probe labels include fluorophores, isotopes, mass tags, or combinations thereof.

Various methods can be used to detect nucleic acid sequences. In some embodiments, detection of an RNA molecule is carried out by detection of nucleic acid sequence templated from the RNA or a cDNA of the RNA by sequencing by synthesis (SBS), sequencing by ligation (SBL), or sequencing by hybridization (SBH). In some embodiments, the detection of targeted RNA species is carried out by the detection of nucleic acid sequence contained in the probe, the probe set or nucleic acid component of the probe complex, by sequencing by synthesis (SBS), sequencing by ligation (SBL), or sequencing by hybridization (SBH); e.g. “barcode” sequencing. In some other embodiments, the detection of the targeted RNA species comprises detection of both nucleic acid sequence templated from RNA or cDNA and nucleic acid sequence contained in the probe, the probe set or nucleic acid component of the probe complex, sequencing by synthesis (SBS), sequencing by ligation (SBL), or sequencing by hybridization (SBH).

After the hybridization, ligation and/or amplification of the polynucleotide probes, the amplification product (amplicon) and/or other portions of the polynucleotide probe set, such as the one or more barcode(s) present in the amplification product and/or polynucleotide probe set according to any of the methods described herein, the barcodes can be analyzed via sequencing to identify the target nucleic acids.

In some embodiments, detection and/or sequencing can be performed in situ, on the intact sample and/or in an embedded sample that preserves the spatial information. In some aspects, the provided embodiments involve in situ analysis and/or sequence determination of the one or more barcode(s) present in the amplification product and/or polynucleotide probe, or a portion thereof, to ultimately identify the presence, level, and/or location of the target nucleic acid (e.g., one or more mRNAs) in the sample.

A wide variety of different sequencing methods can be used to analyze the barcodes. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

In some embodiments, sequencing can be performed in situ. In some embodiments, the in situ sequencing is performed on a cell or a biological sample, such as a tissue sample, that is embedded in a scaffold or a matrix. In some embodiments, the in situ sequencing is performed after amplification of polynucleotides in the probe set and anchoring or cross-linking of the amplification products (e.g., nanoball). In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequencing/sequence detection are described, for example, in Mitra et al., (2003) Anal. Biochem. 320, 55-65, and Lee et al., (2014) Science, 343(6177), 1360-1363. In addition, examples of methods and systems for performing in situ sequencing/sequence detection are described in WO2014/163886, WO2018/045181, WO2018/045186, and in U.S. Pat. Nos. 10,138,509 and 10,179,932. Exemplary techniques for in situ sequencing/sequence detection include, but are not limited to, STARmap (described for example in Wang et al., (2018) Science, 361(6499) 5691), MERFISH (described for example in Moffitt, (2016) Methods in Enzymology, 572, 1-49), and FISSEQ (described for example in US 2019/0032121).

In some embodiments, sequencing can be performed by sequencing-by-synthesis (SBS). In some embodiments, a sequencing primer is complementary to sequences at or near the one or more barcode(s). In such embodiments, sequencing-by-synthesis can include reverse transcription and/or amplification in order to generate a template sequence from which a primer sequence can bind. Exemplary SBS methods include those described for example, but not limited to, US 2007/0166705, US 2006/0188901, U.S. Pat. No. 7,057,026, US 2006/0240439, US 2006/0281109, WO 05/065814, US 2005/0100900, WO 06/064199, WO07/010,251, US 2012/0270305, US 2013/0260372, and US 2013/0079232.

In some embodiments, sequencing can be performed by sequential fluorescence hybridization (e.g., sequencing by hybridization). Sequential fluorescence hybridization can involve sequential hybridization of detection probes including an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597. In some embodiments, the barcodes of the detection probes are targeted by detectably labeled detection oligonucleotides, such as fluorescently labeled oligonucleotides. In some embodiments, one or more decoding schemes are used to decode the signals, such as fluorescence, for sequence determination. Exemplary decoding schemes are described in Eng et al., Nature 568(7751):235-239 (2019); Chen et al., Science; 348(6233):aaa6090 (2015); U.S. Pat. No. 10,457,980; US 2016/0369329 WO 2018/026873 and US 2017/0220733, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, an exemplary method for analysis and/or sequence determination the amplified barcodes include a method that employs a pair of detection probes, such detection probes for sequencing-by-ligation (SBL). In some aspects, the analysis and/or identification involves SBL. In some aspects, a sample, such as a cell or a biological sample, comprising the target nucleic acids and amplification products of a second polynucleotide comprising one or more barcode(s), is contacted with a pair of detection probes, e.g., for SBL, to determine the sequence of the one or more barcode(s). In some embodiments, the amplified second polynucleotide is generated by contacting the sample with the polynucleotide probe set to form hybridization complexes, ligating the second polynucleotide and amplifying the second polynucleotide, generally as described herein. In some aspects, the analysis and/or sequence determination involves contacting a sample containing the amplification products (amplicon) having the barcode sequences with a pair of detection probes, for example, for SBL, under conditions to allow for ligation, wherein the pair of detection probes include a first detection probe and a second detection probe, wherein the ligation only occurs when both the first detection probe and the second detection probe ligate to the same amplicon.

In some aspects, the analysis and/or sequence determination can be carried out at room temperature for best preservation of tissue morphology with low background noise and error reduction. In some embodiments, the analysis and/or sequence determination includes eliminating error accumulation as sequencing proceeds.

In some embodiments, the contacting the amplification products with the first detection probe and the second detection probe, for example, for SBL, occurs two times or more, including, but not limited to, e.g., three times or more, four times or more, five times or more, six times or more, or seven times or more. In some embodiments, the contacting the amplification products occurs four times or more for thin tissue specimens. In some embodiments, the contacting the amplification products occurs six times or more for thick tissue specimens. In some embodiments, one or more amplicons can be contacted by a pair of detection probes, for example, for SBL, for 24 or more hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes.

In some aspects, samples may be analyzed by any of a number of different types of microscopy, for example, optical microscopy (e.g., bright field, oblique illumination, dark field, phase contrast, differential interference contrast, interference reflection, epifluorescence, confocal, etc., microscopy), laser microscopy, electron microscopy, and scanning probe microscopy. In some aspects, a non-transitory computer readable medium transforms raw images acquired through microscopy of multiple rounds of in situ sequencing first into decoded gene identities and spatial locations and then analyzes the per-cell composition of gene expression.

In some embodiments, the detection probes, for example, for SBL, include a first detection probe and a second detection probe. In some aspects, for SBL, the first detection probe is configured to decode bases and the second detection probe is configured to convert decoded bases into a signal. In some aspects, the signal is a fluorescent signal. In exemplary aspects, the contacting the amplification products having the barcode sequence with a pair of detection probes under conditions to allow for ligation involves each of the first detection probe and the second detection probe ligating to form a stable product for imaging only when a perfect match occurs. In some aspects, the mismatch sensitivity of a ligase enzyme is used to determine the underlying sequence of the target nucleic acid molecule.

The term “perfectly matched”, when used in reference to a duplex means that the polynucleotide and/or oligonucleotide strands making up the duplex form a double stranded structure with one another such that every nucleotide in each strand undergoes Watson-Crick base pairing with a nucleotide in the other strand. The term “duplex” includes, but is not limited to, the pairing of nucleoside analogs, such as deoxyinosine, nucleosides with 2-aminopurine bases, peptide nucleic acids (PNAs), and the like, that may be employed. A “mismatch” in a duplex between two polynucleotides and/or oligonucleotides means that a pair of nucleotides in the duplex fails to undergo Watson-Crick bonding.

In some embodiments, detection probes, for example, for SBL, can have sequences with formula NₓB_{y}N_{z}, wherein N is an unknown degenerate base; B is a known interrogatory base; and x, y, and z are integers independent of each other, wherein x and/or z equal zero or greater and y equals one or greater. In some embodiments, x and/or z equals 0. In some embodiments, x and/or z equals 1. In some embodiments, x and/or z equals 2. In some embodiments, x and/or z equals 4. In some embodiments, x and/or z equals 6. In some embodiments, x and/or z equals 8. In some embodiments, x and/or z equals 10. In some embodiments, x and/or z equals 12. In some embodiments, x and/or z equals 14. In some embodiments, x and/or z equals 16. In some embodiments, x and/or z equals 18. In some embodiments, x and/or z equals 20. In some embodiments, y equals 1. In some embodiments, y equals 2. In some embodiments, y equals 3. In some embodiments, y equals 4. In some embodiments, y equals 5. In some embodiments, y equals 6. In some embodiments, y equals 7. In some embodiments, y equals 8. In some embodiments, y equals 9. In some embodiments, y equals 10. In some embodiments, y equals 12. In some embodiments, y equals 14. In some embodiments, y equals 16. In some embodiments, y equals 18. In some embodiments, y equals 20.

In some embodiments, the analysis and/or sequence determination, for example, by SBL, involves a ligase with activity hindered by base mismatches, a first detection probe, and a second detection probe. The term “hindered” in this context refers to activity of a ligase that is reduced by approximately 20% or more, such as by 25% or more, such as by 50% or more, such as by 75% or more, such as by 90% or more, such as by 95% or more, such as by 99% or more, such as by 100%. In some embodiments, the first detection probe has a length of 5-15 nucleotides, including, but not limited to, 5-13 nucleotides, 5-10 nucleotides, or 5-8 nucleotides. In some embodiments, the Tₘ of the first detection probe is at room temperature (in some examples, less than 30° C., or 22-25° C.). In some embodiments, the first detection probe is degenerate, or partially thereof. In some embodiments, the second detection probe has a length of 5-15 nucleotides, including, but not limited to, 5-13 nucleotides, 5-10 nucleotides, or 5-8 nucleotides. In some embodiments, the Tₘ of the second detection probe is at room temperature (in some examples, less than 30° C., or 22-25° C.). After each cycle of contacting corresponding to a base readout, the second detection probes may be stripped, which eliminates error accumulation as sequencing proceeds. In some embodiments, the second detection probes are stripped by formamide.

In some embodiments, the analysis and/or sequence determination, for example, by SBL, involves the washing of the first detection probe and the second detection probe to remove unbound detection probes, thereafter revealing a fluorescent product for imaging. In some embodiments, a detectable label can be used to detect one or more nucleotides and/or detection probes, described herein.

In some aspects, the methods involve sequencing nucleic acid in situ within a matrix. Exemplary general sequencing methods known, such as sequencing by extension with reversible terminators, fluorescent in situ sequencing (FISSEQ), pyrosequencing, massively parallel signature sequencing (MPSS) (described in Shendure et al. (2004) Nat. Rev. 5:335, incorporated herein by reference in its entirety), are suitable for use with the matrix in which the nucleic acids are present. Reversible termination methods use step-wise sequencing-by-synthesis biochemistry that coupled with reversible termination and removable fluorescence (Shendure et al. supra and U.S. Pat. Nos. 5,750,341 and 6,306,597, incorporated herein by reference. FISSEQ is a method whereby DNA is extended by adding a single type of fluorescently-labelled nucleotide triphosphate to the reaction, washing away unincorporated nucleotide, detecting incorporation of the nucleotide by measuring fluorescence, and repeating the cycle. At each cycle, the fluorescence from previous cycles is bleached or digitally subtracted or the fluorophore is cleaved from the nucleotide and washed away. FISSEQ is described further in Mitra et al. (2003) Anal. Biochem. 320:55, incorporated herein by reference in its entirety for all purposes. Pyrosequencing is a method in which the pyrophosphate (PPi) released during each nucleotide incorporation event (i.e., when a nucleotide is added to a growing polynucleotide sequence). The PPi released in the DNA polymerase-catalyzed reaction is detected by ATP sulfurylase and luciferase in a coupled reaction which can be visibly detected. The added nucleotides are continuously degraded by a nucleotide-degrading enzyme. After the first added nucleotide has been degraded, the next nucleotide can be added. As this procedure is repeated, longer stretches of the template sequence are deduced. Pyrosequencing is described further in Ronaghi et al. (1998) Science 281:363, incorporated herein by reference in its entirety for all purposes. MPSS utilizes ligation-based DNA sequencing simultaneously on microbeads. A mixture of labelled adaptors comprising all possible overhangs is annealed to a target sequence of four nucleotides. The label is detected upon successful ligation of an adaptor. A restriction enzyme is then used to cleave the DNA template to expose the next four bases. MPSS is described further in Brenner et al. (2000) Nat. Biotech. 18:630, incorporated herein by reference in its entirety for all purposes.

In some aspects, the nucleic acids within the matrix can be interrogated using methods known to those of skill in the art including fluorescently labeled oligonucleotide/DNA/RNA hybridization, primer extension with labeled ddNTP, sequencing by ligation and sequencing by synthesis. Ligated circular padlock probes described in Larsson, et al., (2004), Nat. Methods 1:227-232 can be used to detect multiple sequence targets in parallel, followed by either sequencing-by-ligation (SBL), -synthesis or -hybridization of the barcode sequences in the padlock probe to identify individual targets.

### V. SAMPLES, ANALYTES AND TARGET SEQUENCES FOR ANALYSIS

In some aspects, the provided methods are used to analyze target nucleic acids present in a sample or a specimen, such as in a cell or a tissue sample. In some aspects, the target nucleic acids are present in a sample or a specimen among a plurality of different nucleic acids. In some aspects, the sample is a biological sample, is derived from a biological sample or is a biological sample that was subject to various processes or manipulations. In some aspects, the provided methods also involve processing or manipulating the sample, before and/or after the hybridization, ligation, amplification and/or detection steps, to facilitate the analysis of the target nucleic acids and/or to preserve the spatial information of the target nucleic acid within the sample or specimen.

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, the provided methods also involve embedding the target nucleic acid or a biological sample containing the target nucleic acid with a scaffold, a matrix or a network. In some embodiments, the provided methods also involve anchoring or cross-linking the target nucleic acid, one or more polynucleotide(s) of the polynucleotide probe set and/or an amplification product, to a scaffold, to cellular structures, to other probe polynucleotides and/or to other amplification products. In some aspects, the provided methods can be applied as for a three-dimensional (3D) in situ transcriptomic analysis. In some aspects, the provided embodiments can be used to detect, sequence and/or analyze the presence of a plurality of different target nucleic acids in a sample in a 3D space with high sensitivity and reduced analysis time and provides versatility for anchoring and/or functionalizing part of the polynucleotide probe sets for further processes or assessment. In addition, the provided methods can be used in iterative steps, with several rounds of detection and/or sequencing analysis, or application with other reagents for comprehensive analysis.

Biological samples can include analytes (e.g., nucleic acids, such as RNA, and/or DNA, and/or protein) embedded in a matrix, such as a 3D matrix. In some embodiments, amplicons (e.g., rolling circle amplification products) derived from or associated with analytes (e.g., nucleic acids, such as RNA, and/or DNA, and/or protein) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel. Exemplary methods and techniques for generating a matrix, in connection with the provided methods, include those described herein.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

A biological sample may comprise one or a plurality of analytes of interest, for example, a plurality of target nucleic acids. Methods for performing multiplexed assays to analyze two or more different analytes in a single biological sample are provided.

A. Cells

In some aspects, the provided embodiments include a method for analyzing a target nucleic acid in a cell or a biological sample. In some aspects, the provided embodiments include in situ gene sequencing of a target nucleic acid in a cell in an intact tissue. In some embodiments, the cell is present in a population of cells. In some embodiments, the population of cells includes a plurality of cell types including, but not limited to, excitatory neurons, inhibitory neurons, and non-neuronal cells. Cells for use in the assays of the provided embodiments can be an organism, a single cell type derived from an organism, or can be a mixture of cell types. Included are naturally occurring cells and cell populations, genetically engineered cell lines, cells derived from transgenic animals, etc. Virtually any cell type and size can be accommodated. Suitable cells include bacterial, fungal, plant and animal cells. In some embodiments, the cells are mammalian cells, e.g., complex cell populations such as naturally occurring tissues, for example blood, liver, pancreas, neural tissue, bone marrow or skin. Some tissues may be disrupted into a monodisperse suspension. In some embodiments, the cells may be a cultured population, e.g., a culture derived from a complex population, a culture derived from a single cell type where the cells have differentiated into multiple lineages, or where the cells are responding differentially to stimulus.

In some aspects, cell types to be used in accordance with the provided embodiments include stem and progenitor cells, e.g., embryonic stem cells, hematopoietic stem cells, mesenchymal stem cells, neural crest cells, endothelial cells, muscle cells, myocardial, smooth and skeletal muscle cells, mesenchymal cells, epithelial cells, hematopoietic cells, such as lymphocytes, including T cells, such as helper T cells, Th1 cells, Th2 cells, Th0 cells, cytotoxic T cells, regulatory T cells, B cells, pre-B cells, monocytes, dendritic cells, neutrophils, and macrophages, natural killer cells, mast cells, adipocytes, cells involved with particular organs, such as thymus, endocrine glands, pancreas, brain, such as neurons, glia, astrocytes, dendrocytes and genetically modified cells thereof. Hematopoietic cells may be associated with inflammatory processes, autoimmune diseases, endothelial cells, smooth muscle cells, myocardial cells may be associated with cardiovascular diseases, almost any type of cell may be associated with neoplasias, such as sarcomas, carcinomas and lymphomas, liver diseases with hepatic cells, kidney diseases with kidney cells.

In some aspects, the cells may also be transformed or neoplastic cells of different types, e.g., carcinomas of different cell origins, lymphomas of different cell types. For example, the American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

In some aspects, the cells may be non-adherent, e.g., blood cells including monocytes, T cells, B-cells; tumor cells, etc., or adherent cells, e.g., epithelial cells, endothelial cells, neural cells, etc. For assessment of adherent cells, they may be dissociated from the substrate that they are adhered to, and from other cells, in a manner that maintains their ability to recognize and bind to probe molecules. In some aspects, such cells can be acquired from an individual using, e.g., a draw, a lavage, a wash, surgical dissection etc., from a variety of tissues, e.g., blood, marrow, a solid tissue (e.g., a solid tumor), ascites, by a variety of techniques that are known. In some aspects, cells may be obtained from fixed or unfixed, fresh or frozen, whole or disaggregated samples or tissues, for example, disaggregated mechanically or enzymatically.

In some aspects, the methods include permeabilizing and/or fixing the sample, such as the cell.

B. Tissue Samples

In some aspects, the analyte, e.g., the target nucleic acid, for analysis in accordance with the provided embodiments is analyzed in the context of a tissue sample, for example, in situ in a tissue sample. In some aspects, the tissue sample is a tissue section. In some embodiments, the tissue sample is an intact tissue sample or a non-homogenized tissue sample. In some embodiments, the target nucleic acid is in a cell in the tissue sample.

1. Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

In some aspects, the provided methods involve in situ detection and/or sequencing of an intact tissue, or a sample derived from an intact tissue. In some aspects, the provided embodiments involve contacting a fixed and permeabilized intact tissue with one or more polynucleotide probe sets as described herein under conditions to allow for specific hybridization to the target nucleic acid. In some aspects, tissue specimens suitable for use in accordance with the provided embodiments generally include any type of tissue specimens collected from living or dead subjects, such as, e.g., biopsy specimens and autopsy specimens, of which include, but are not limited to, epithelium, muscle, connective, and nervous tissue. In some embodiments, tissue specimens may be collected and processed in accordance with the provided embodiments and subjected to microscopic analysis immediately following processing, or may be preserved and subjected to microscopic analysis at a future time, e.g., after storage for an extended period of time. In some embodiments, the provided embodiments may be used to preserve tissue specimens in a stable, accessible and fully intact form for future analysis. In some embodiments, the provided embodiments may be used to analyze a previously-preserved or stored tissue specimen. In some embodiments, the intact tissue includes brain tissue such as visual cortex slices. In some embodiments, the intact tissue is a thin slice with a thickness of 5-20 μm, including, but not limited to, e.g., 5-18 μm, 5-15 μm, or 5-10 μm. In some embodiments, the intact tissue is a thick slice with a thickness of 50-200 μm, including, but not limited to, e.g., 50-150 μm, 50-100 μm, or 50-80 μm.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

2. Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

3. Fixation and Postfixation

In some embodiments, the methods involve permeabilizing and/or fixing the sample, such as the cell or the tissue sample. In some embodiments, the tissue sample is a fixed tissue sample, e.g., a formalin-fixed, paraffin-embedded (FFPE) sample, a frozen tissue sample, or a fresh tissue sample.

In some aspects, the methods include fixing intact tissue. The term “fixing” or “fixation” as used herein is the process of preserving biological material (e.g., tissues, cells, organelles, molecules, etc.) from decay and/or degradation. Fixation may be accomplished using any convenient protocol. Fixation can include contacting the sample with a fixation reagent (i.e., a reagent that contains at least one fixative). Samples can be contacted by a fixation reagent for a wide range of times, which can depend on the temperature, the nature of the sample, and on the fixative(s). For example, a sample can be contacted by a fixation reagent for 24 hours or less, 18 hours or less, 12 hours or less, 8 hours or less, 6 hours or less, 4 hours or less, 2 hours or less, 60 minutes or less, 45 minutes or less, 30 minutes or less, 25 minutes or less, 20 minutes or less, 15 minutes or less, 10 minutes or less, 5 minutes or less, or 2 minutes or less.

In some embodiments, a sample can be contacted by a fixation reagent for a period of time in a range of from 5 minutes to 24 hours, e.g., from 10 minutes to 20 hours, from 10 minutes to 18 hours, from 10 minutes to 12 hours, from 10 minutes to 8 hours, from 10 minutes to 6 hours, from 10 minutes to 4 hours, from 10 minutes to 2 hours, from 15 minutes to 20 hours, from 15 minutes to 18 hours, from 15 minutes to 12 hours, from 15 minutes to 8 hours, from 15 minutes to 6 hours, from 15 minutes to 4 hours, from 15 minutes to 2 hours, from 15 minutes to 1.5 hours, from 15 minutes to 1 hour, from 10 minutes to 30 minutes, from 15 minutes to 30 minutes, from 30 minutes to 2 hours, from 45 minutes to 1.5 hours, or from 55 minutes to 70 minutes.

In some embodiments, a sample can be contacted by a fixation reagent at various temperatures, depending on the protocol and the reagent used. For example, in some instances a sample can be contacted by a fixation reagent at a temperature ranging from −22° C. to 55° C., where specific ranges of interest include, but are not limited to 50 to 54° C., 40 to 44° C., 35 to 39° C., 28 to 32° C., 20 to 26° C., 0 to 6° C., and −18 to −22° C. In some instances a sample can be contacted by a fixation reagent at a temperature of −20° C., 4° C., room temperature (in some examples, less than 30° C., or 22-25° C.), 30° C., 37° C., 42° C., or 52° C.

In some embodiments, any known fixation reagent can be used. Common fixation reagents include cross-linking fixatives, precipitating fixatives, oxidizing fixatives, mercurials, and the like. Cross-linking fixatives chemically join two or more molecules by a covalent bond and a wide range of cross-linking reagents can be used. Examples of suitable cross liking fixatives include but are not limited to aldehydes (e.g., formaldehyde, also commonly referred to as “paraformaldehyde” and “formalin”; glutaraldehyde; etc.), imidoesters, NHS (N-Hydroxysuccinimide) esters, and the like. Examples of suitable precipitating fixatives include but are not limited to alcohols (e.g., methanol, ethanol, etc.), acetone, acetic acid, etc. In some embodiments, the fixative is formaldehyde (i.e., paraformaldehyde or formalin). A suitable final concentration of formaldehyde in a fixation reagent is 0.1 to 10%, 1-8%, 1-4%, 1-2%, 3-5%, or 3.5-4.5%, including about 1.6% for 10 minutes. In some embodiments the sample is fixed in a final concentration of 4% formaldehyde (as diluted from a more concentrated stock solution, e.g., 38%, 37%, 36%, 20%, 18%, 16%, 14%, 10%, 8%, 6%, etc.). In some embodiments the sample is fixed in a final concentration of 10% formaldehyde. In some embodiments the sample is fixed in a final concentration of 1% formaldehyde. In some embodiments, the fixative is glutaraldehyde. A suitable concentration of glutaraldehyde in a fixation reagent is 0.1 to 1%. A fixation reagent can contain more than one fixative in any combination. For example, in some embodiments the sample is contacted with a fixation reagent containing both formaldehyde and glutaraldehyde.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

4. Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

5. Staining

To facilitate visualization, biological samples can be stained using a wide variety of stains and staining techniques. In some embodiments, for example, a sample can be stained using any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, propidium iodide, rhodamine, or safranine.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample are known in the art, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

6. Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

7. Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix, e.g., as described in Section V.D. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

8. Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

The terms “permeabilization” or “permeabilize” as used herein refer to the process of rendering the cells (cell membranes etc.) of a sample permeable to experimental reagents such as nucleic acid probes, antibodies, chemical substrates, etc. Any convenient method and/or reagent for permeabilization can be used. Suitable permeabilization reagents include detergents (e.g., Saponin, Triton X-100, Tween-20, etc.), organic fixatives (e.g., acetone, methanol, ethanol, etc.), enzymes, etc. Detergents can be used at a range of concentrations. For example, 0.001%-1% detergent, 0.05%-0.5% detergent, or 0.1%-0.3% detergent can be used for permeabilization (e.g., 0.1% Saponin, 0.2% tween-20, 0.1-0.3% triton X-100, etc.). In some embodiments methanol on ice for at least 10 minutes is used to permeabilize.

In some embodiments, the same solution can be used as the fixation reagent and the permeabilization reagent. For example, in some embodiments, the fixation reagent contains 0.1%-10% formaldehyde and 0.001%-1% saponin. In some embodiments, the fixation reagent contains 1% formaldehyde and 0.3% saponin.

In some embodiments, a sample can be contacted by a permeabilization reagent for a wide range of times, which can depend on the temperature, the nature of the sample, and on the permeabilization reagent(s). For example, a sample can be contacted by a permeabilization reagent for 24 or more hours, 24 hours or less, 18 hours or less, 12 hours or less, 8 hours or less, 6 hours or less, 4 hours or less, 2 hours or less, 60 minutes or less, 45 minutes or less, 30 minutes or less, 25 minutes or less, 20 minutes or less, 15 minutes or less, 10 minutes or less, 5 minutes or less, or 2 minutes or less. In some embodiments, a sample can be contacted by a permeabilization reagent at various temperatures, depending on the protocol and the reagent used. For example, in some instances a sample can be contacted by a permeabilization reagent at a temperature ranging from −82° C. to 55° C., where specific ranges of interest include, but are not limited to: 50 to 54° C., 40 to 44° C., 35 to 39° C., 28 to 32° C., 20 to 26° C., 0 to 6° C., −18 to −22° C., and −78 to −82° C. In some instances a sample can be contacted by a permeabilization reagent at a temperature of −80° C., −20° C., 4° C., room temperature (in some examples, less than 30° C., or 22-25° C.), 30° C., 37° C., 42° C., or 52° C.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

In some embodiments, a sample is contacted with an enzymatic permeabilization reagent. Enzymatic permeabilization reagents that permeabilize a sample by partially degrading extracellular matrix or surface proteins that hinder the permeation of the sample by assay reagents. Contact with an enzymatic permeabilization reagent can take place at any point after fixation and prior to target detection. In some instances the enzymatic permeabilization reagent is proteinase K, a commercially available enzyme.

In such cases, the sample is contacted with proteinase K prior to contact with a post fixation reagent. Proteinase K treatment (i.e., contact by proteinase K; also commonly referred to as “proteinase K digestion”) can be performed over a range of times at a range of temperatures, over a range of enzyme concentrations that are empirically determined for each cell type or tissue type under investigation. For example, a sample can be contacted by proteinase K for 30 minutes or less, 25 minutes or less, 20 minutes or less, 15 minutes or less, 10 minutes or less, 5 minutes or less, or 2 minutes or less. A sample can be contacted by 1 pg/ml or less, 2 pg/ml or less, 4 pg/ml or less, 8 pg/ml or less, 10 pg/ml or less, 20 pg/ml or less, 30 pg/ml or less, 50 pg/ml or less, or 100 pg/ml or less proteinase K. In some embodiments, a sample can be contacted by proteinase K at a temperature ranging from 2° C. to 55° C., where specific ranges of interest include, but are not limited to: 50 to 54° C., 40 to 44° C., 35 to 39° C., 28 to 32° C., 20 to 26° C., and 0 to 6° C. In some instances a sample can be contacted by proteinase K at a temperature of 4° C., room temperature (in some examples, less than 30° C., or 22-25° C.), 30° C., 37° C., 42° C., or 52° C. In some embodiments, a sample is not contacted with an enzymatic permeabilization reagent. In some embodiments, a sample is not contacted with proteinase K. In some aspects, contact of an intact tissue with at least a fixation reagent and a permeabilization reagent results in the production of a fixed and permeabilized tissue.

9. Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

C. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected. In some aspects, the analyte is a nucleic acid, such as a target nucleic acid.

A target sequence for a probe disclosed herein (e.g., polynucleotide probe set) may be comprised in any analyte disclose herein, including an endogenous analyte (e.g., a viral or cellular nucleic acid), a labelling agent, or a product of an endogenous analyte and/or a labelling agent.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

1. Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target nucleic acid sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

2. Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

3. Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product thereof is analyzed. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product) of a labelling agent that directly or indirectly binds to an analyte in the biological sample is analyzed. In some embodiments, the products comprise sequences for hybridizing to the first, second, and third polynucleotides of the probe sets described herein.

D. Anchoring or Linkage of Amplification Products

In some aspects, one or more polynucleotide of the probe set, for example at least a part of the first polynucleotide, the second polynucleotide or the third polynucleotide, can contain one or more anchoring site(s) and/or hybridization site(s) to anchor, link, or attach the polynucleotide probe set and/or the amplification product (e.g., amplified circularized second polynucleotides, or “nanoballs”), to a scaffold (e.g., matrix), to cellular structures, to other probe polynucleotides and/or to other amplification products. In some embodiments, the methods involve comprising cross-linking the target nucleic acid and/or the amplification product to a matrix. In some embodiments, the methods involve comprising cross-linking the target nucleic acid and/or the amplification product to another target nucleic acid and/or amplification product. In some embodiments, the biological sample is reversibly cross-linked.

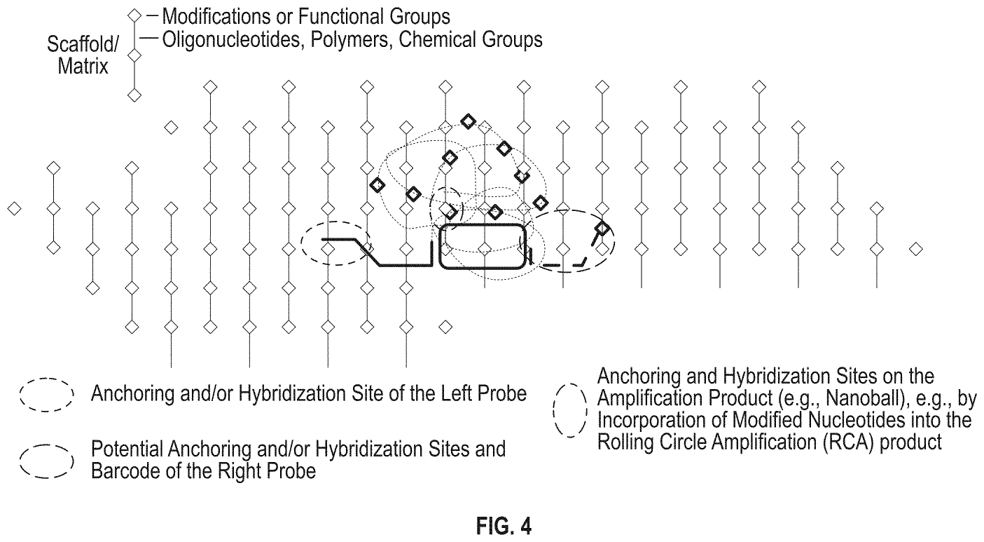

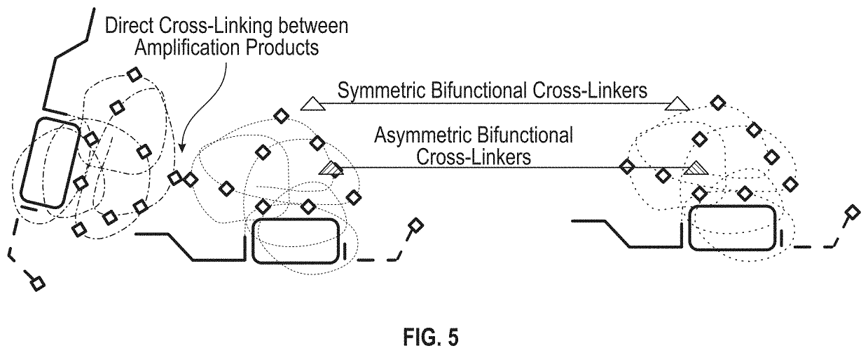

In some aspects, one or more polynucleotide(s) of the probe set can contain one or more modification(s) that can be anchoring site(s) and/or hybridization site(s). For example, the one or more modification(s) can be on the first polynucleotide, the second polynucleotide or the third polynucleotide, or an amplified product (e.g., amplified product of the second polynucleotide). In some aspects, exemplary positions that can contain modifications for anchoring sites and/or hybridization sites are depicted in FIG. 2. In some aspects, the anchoring sites and/or hybridization sites can be used to anchor the polynucleotide probe set, amplified product and/or target nucleic acid to a scaffold or a matrix. In some aspects, the matrix comprises a hydrogel. In some embodiments, the matrix comprises one or more nucleic acids and the hybridization site(s) on the polynucleotide probes may hybridize to the nucleic acid matrix. Schematics that depict exemplary anchoring and/or hybridization sites and anchoring and/or attachment of the polynucleotide probe set, amplified product and/or target nucleic acid to a scaffold or a matrix (FIG. 4) or other polynucleotide probe set, amplified product and/or target nucleic acid (FIG. 5) are shown in FIGS. 4-5.

In some aspects, the sample and/or the target nucleic acid is contacted with a plurality of polynucleotide probe sets, for example that comprise a second polynucleotide. In some aspects, the second polynucleotide is amplified using known methods, such as isothermal enzymatic amplification, such as rolling circle amplification (RCA). In some embodiments, the amplification products are localized near the second polynucleotide. In some aspects, the amplification products form a shell (e.g., nanoball) around the second polynucleotide or otherwise assemble around the second polynucleotide. Each second polynucleotide may have more than 1000 amplification products surrounding or otherwise associated therewith. In some aspects, the amplification products surrounding a particular second polynucleotide provide a high signal intensity, due in part to the number of amplification products and/or detectable labels associated with the amplification products. In some embodiments, a plurality the amplification products, e.g., amplified second polynucleotides or nanoballs, can be attached or linked to one another. In some embodiments, the amplification products may be functionalized and cross-linked or otherwise covalently bound together around their associate second polynucleotide to form a series or network of tightly bound DNA amplification product shells (e.g., nanoballs) around each second polynucleotide. The series or network of tightly bound DNA amplification product shells around each second polynucleotide may be assembled onto a three-dimensional support. In some aspects, the series or network of tightly bound DNA amplification product shells around each second polynucleotide may be assembled onto a three-dimensional scaffold or a matrix producing a three dimensional DNA polymer with defined overall shape, size and amplification product position.

As used herein, the term “attach” can be either covalent interactions or noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994, incorporated herein by reference in its entirety for all purposes.

In some embodiments, the polynucleotide probe set, amplified product (e.g., nanoball) and/or target nucleic acid to another nucleic acid molecule (another polynucleotide probe set, amplified product (e.g., nanoball) and/or target nucleic acid) containing the anchoring site(s) and/or hybridization site(s) can be anchored or cross-linked by direct cross-linking.

In some embodiments, the amplification products (amplicons) are covalently linked without the need for separate cross-linkers, such as bis-N-succinimidyl-(nonaethylene glycol) ester. In some embodiments, an acrydite moiety, such as a catalyst activated acrydite moiety is introduced at the end of a long carbon spacer (i.e., about C6 to about C12) at position 5 of a uracil base a representative formula of which is shown below.

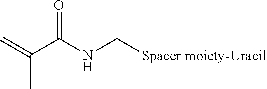

In the formula below, R represents the acrydite spacer moiety attached to the 5 position of the uracil base.

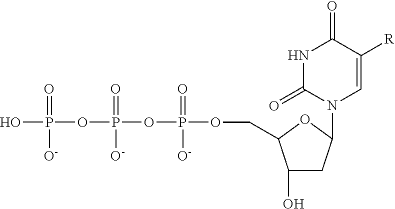

In some aspects, when copolymerized with bis-acrylamide in the presence of a catalyst, a polymerization reaction takes place, encapsulating the second polynucleotide with the amplicons and fixing the amplicons in position. In some aspects, the chemically inert nature of the polymerized mixture allows various downstream applications. In some aspects, the spacer can be a carbon chain of between about 2 carbons to about 200 carbons. In some embodiments, the can be polyethylene glycol. The length of In some embodiments, the can vary from about 30 angstroms to about 100 angstroms and can be of various molecular weights. In some embodiments, the can be permanent or reversible, such as by using UV light, enzymes, chemical cleavage, etc. In some aspects, a three dimensional matrix, such as a polyacrylamide gel matrix, can be used to embed a variety of biological structures containing enzymatically or chemically modified DNA or RNA molecules containing an acrydite functional moiety or moieties. In some embodiments, the non-nucleic acid component is selectively dissolved using detergents, proteases, organic solvents or denaturants to create a three dimensional matrix that preserves individual DNA or RNA molecules and their relative spatial location. Examples include embedding cells, healthy and diseased tissues and tissue sections, small model organisms such as worms and insects, bacterial colonies or biofilm, environmental samples containing other DNA or RNA containing materials or organisms.

In some aspects, the polynucleotide probes and/or amplification products are modified to incorporate a functional moiety for attachment to the matrix. The functional moiety can be covalently cross-linked, copolymerize with or otherwise non-covalently bound to the matrix. The functional moiety can react with a cross-linker. The functional moiety can be part of a ligand-ligand binding pair. dNTP or dUTP can be modified with the functional group, so that the function moiety is introduced into the DNA during amplification. A suitable exemplary functional moiety includes an amine, acrydite, alkyne, biotin, azide, and thiol. In the case of cross-linking, the functional moiety is cross-linked to modified dNTP or dUTP or both. Suitable exemplary cross-linker reactive groups include imidoester (DMP), succinimide ester (NHS), maleimide (Sulfo-SMCC), carbodiimide (DCC, EDC) and phenyl azide.

In some aspects, the anchoring site(s) and/or hybridization site(s) can be anchored or attached via one or more cross-linker(s). Cross-linking can be performed chemically and/or photochemically, or alternatively by any other hydrogel-formation method known in the art. In some embodiments, the polynucleotide probe set, amplified product (e.g., nanoball) and/or target nucleic acid to another nucleic acid molecule (another polynucleotide probe set, amplified product (e.g., nanoball) and/or target nucleic acid) containing the anchoring site(s) and/or hybridization site(s) can be anchored or cross-linked by using one or more cross-linker(s). Cross-linkers can be used to anchor or cross-link different nanoballs at a distance, or to other cellular structures or scaffolding. In some embodiments, the cross-linker is a symmetric bifunctional cross-linker. In some embodiments, the cross-linker is an asymmetric bifunctional cross-liner.

Cross-linkers within the scope of the present disclosure may include a spacer moiety. Such spacer moieties may be functionalized. Such spacer moieties may be chemically stable. Such spacer moieties may be of sufficient length to allow amplification of the nucleic acid bound to the matrix. Suitable exemplary spacer moieties include polyethylene glycol, carbon spacers, photo-cleavable spacers and other spacers known and the like. The polynucleotide probes and/or amplification products can include functional groups necessary for structural interaction with components of a polymer matrix, cellular structures or components, for example, proteins, and/or with another polynucleotide probe and/or amplification product, for example via hydrogen bonding. Exemplary functional groups can include at least an amine, carbonyl, hydroxyl or carboxyl group, or at least two of the functional chemical groups. The polynucleotide probes and/or amplification products can include cyclical carbon or heterocyclic structures and/or aromatic or polyaromatic structures substituted with one or more of the above functional groups.

In some aspects of the provided methods, a rolling circle amplification product is formed using the first polynucleotide as a primer and the circularized second polynucleotide as a template. In some aspects, the rolling circle amplification product forms a DNA nanoball. In some aspects, two or more rolling circle amplification products (e.g., DNA nanoballs) are formed. In some aspects, the two or more rolling circle amplification products (e.g., DNA nanoballs) are directly cross-linked, cross-linked via a symmetric bi-functional cross-linker, or cross-linked via an asymmetric bi-functional cross-linker.

In some aspects, nucleic acid amplicons, such as amplicons produced in accordance with the disclosure provided herein, can be covalently linked to one another. The circular DNA molecules are then amplified using known methods and/or exemplary methods described herein, for example, RCA. In some aspects, the amplicons are localized near the circular DNA. In some aspects, the amplicons form a shell around the circular DNA or otherwise assemble around the circular DNA. Each circular DNA may have more than 1000 amplicons surrounding or otherwise associated therewith. In some aspects, the amplicons surrounding a particular circular DNA provide a high signal intensity, due in part to the number of amplicons and/or detectable labels associated with the amplicons. The amplicons may be functionalized and cross-linked or otherwise covalently bound together around their associate circular DNA to form a series or network of tightly bound DNA amplicon shells around each circular DNA. The series or network of tightly bound DNA amplicon shells around each circular DNA may be assembled onto a three-dimensional support. In some aspects, the series or network of tightly bound DNA amplicon shells around each circular DNA may be assembled onto a three-dimensional support producing a three dimensional DNA polymer with defined overall shape, size and amplicon position.

1. Matrix

In some aspects, the polynucleotide probes and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix.

Exemplary modification and polymer matrix that can be employed in accordance with the provided embodiments include those described in, for example, WO 2014/163886, WO 2017/079406, US 2016/0024555, US 2018/0251833, US 2017/0219465, and WO2014/025392. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

In some cases, the backbone of the polynucleotide, for example, the amplification product of the second polynucleotide, can include a polymer of synthetic subunits such as phosphoramidites, and/or phosphorothioates, and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. The polynucleotide may include one or more L-nucleosides. A polynucleotide may include modified nucleotides, such as methylated nucleotides and nucleotide analogs, uracil, other sugars, and linking groups such as fluororibose and thioate, and nucleotide branches. The polynucleotide may include non-nucleotide components. A polynucleotide may be modified to include N3′-P5′ (NP) phosphoramidate, morpholino phosphorociamidate (MF), locked nucleic acid (LNA), 2′-0-methoxyethyl (MOE), or 2′-fluoro, arabino-nucleic acid (FANA), which can enhance the resistance of the polynucleotide to nuclease degradation (see, e.g., Faria et al. (2001) Nature Biotechnol. 19:40-44; Toulme (2001) Nature Biotechnol. 19:17-18). A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support. Immunomodulatory nucleic acid molecules can be provided in various formulations, e.g., in association with liposomes or microencapsulated. A polynucleotide used in amplification is generally single-stranded for maximum efficiency in amplification, but may alternatively be double-stranded. If double-stranded, the polynucleotide can first be treated to separate its strands before being used to prepare extension products. This denaturation step is typically affected by heat, but may alternatively be carried out using alkali, followed by neutralization.

In some instances, the provided embodiments include a three dimensional matrix of a plurality of nucleic acids. In some embodiments, provided is a three dimensional matrix containing a plurality of polynucleotides and/or amplification products bound thereto. In some aspects, the matrix is a three dimensional nucleic acid-containing polymer. The polynucleotide probes and/or amplification products may be naturally occurring nucleic acids or non-naturally occurring nucleic acids, such as nucleic acids that have been made using synthetic methods. The polynucleotide probes and/or amplification products in the three dimensional matrix may be ordered or unordered. The polynucleotide probes and/or amplification products in the three dimensional matrix may be present in their natural spatial relationship within a cell or a biological sample, such as a tissue, or an organism. The polynucleotide probes and/or amplification products in the three dimensional matrix may be present in rows and columns within the three dimensional matrix.

In some embodiments, the methods involve embedding the tissue sample in a matrix. In some of any embodiments, the matrix is a hydrogel. In some aspects, a matrix-forming material is contacted to a plurality of nucleic acids spatially arrange in three-dimensions relative to one another.

Matrix forming materials include polyacrylamide, cellulose, alginate, polyamide, cross-linked agarose, cross-linked dextran, cross-linked polyethylene glycol, disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The matrix forming materials can form a matrix by polymerization and/or cross-linking of the matrix forming materials using methods specific for the matrix forming materials and known methods, reagents and conditions.

In some aspects, a matrix-forming material can be introduced into the sample, such as a cell or a tissue. In some cases, the cells are fixed with formaldehyde and then immersed in ethanol to disrupt the lipid membrane. The matrix forming reagents are added to the sample and are allowed to permeate throughout the cell. A polymerization inducing catalyst, UV or functional cross-linkers are then added to allow the formation of a gel matrix. The un-incorporated material is washed out and any remaining functionally reactive group is quenched. Exemplary cells include any cell, human or otherwise, including diseased cells or healthy cells. Certain cells include human cells, non-human cells, human stem cells, mouse stem cells, primary cell lines, immortalized cell lines, primary and immortalized fibroblasts, HeLa cells and neurons.

In some aspects, a matrix-forming material can be used to encapsulate a biological sample, such as a tissue sample. The formalin-fixed embedded tissues on glass slides are incubated with xylene and washed using ethanol to remove the embedding wax. They are then treated with Proteinase K to permeabilized the tissue. A polymerization inducing catalyst, UV or functional cross-linkers are then added to allow the formation of a gel matrix. The un-incorporated material is washed out and any remaining functionally reactive group is quenched. Exemplary tissue samples include any tissue samples of interest whether human or non-human. Such tissue samples include those from skin tissue, muscle tissue, bone tissue, organ tissue and the like. Exemplary tissues include human and mouse brain tissue sections, embryo sections, tissue array sections, and whole insect and worm embryos.

The matrix-forming material forms a three dimensional matrix including the plurality of nucleic acids. In some aspects, the matrix-forming material forms a three dimensional matrix including the plurality of nucleic acids while maintaining the spatial relationship of the polynucleotide probes and/or amplification products. In this aspect, the plurality of nucleic acids are immobilized within the matrix material. The plurality of nucleic acids may be immobilized within the matrix material by co-polymerization of the polynucleotide probes and/or amplification products with the matrix-forming material. The plurality of nucleic acids may also be immobilized within the matrix material by cross-linking of the polynucleotide probes and/or amplification products to the matrix material or otherwise cross-linking with the matrix-forming material. The plurality of nucleic acids may also be immobilized within the matrix by covalent attachment or through ligand-protein interaction to the matrix.

In some aspects, the matrix is porous thereby allowing the introduction of reagents into the matrix at the site of a nucleic acid for amplification of the nucleic acid. A porous matrix may be made according to known methods. In one example, a polyacrylamide gel matrix is co-polymerized with acrydite-modified streptavidin monomers and biotinylated DNA molecules, using a suitable acrylamide:bis-acrylamide ratio to control the cross-linking density. Additional control over the molecular sieve size and density is achieved by adding additional cross-linkers such as functionalized polyethylene glycols. In some aspects, the polynucleotide probes and/or amplification products, which may represent individual bits of information, are readily accessed by oligonucleotides, such as labeled oligonucleotide probes, primers, enzymes and other reagents with rapid kinetics.

In some aspects, the matrix is sufficiently optically transparent or otherwise has optical properties suitable for detection, imaging and/or sequencing, for example, as described herein and/or using standard high-throughput sequencing chemistries and deep three dimensional imaging for high throughput information readout. Exemplary high-throughput sequencing chemistries that utilize fluorescence imaging include ABI SoLiD (Life Technologies), in which a sequencing primer on a template is ligated to a library of fluorescently labeled nonamers with a cleavable terminator. After ligation, the beads are then imaged using four color channels (FITC, Cy3, Texas Red and Cy5). The terminator is then cleaved off leaving a free-end to engage in the next ligation-extension cycle. After all dinucleotide combinations have been determined, the images are mapped to the color code space to determine the specific base calls per template. The workflow is achieved using an automated fluidics and imaging device (i.e., SoLiD 5500 W Genome Analyzer, ABI Life Technologies). Another sequencing platform uses sequencing by synthesis, in which a pool of single nucleotide with a cleavable terminator is incorporated using DNA polymerase. After imaging, the terminator is cleaved and the cycle is repeated. The fluorescence images are then analyzed to call bases for each DNA amplicons within the flow cell (HiSeq, Illumina).

In some aspects, the plurality of nucleic acids may be amplified to produce amplification products (e.g., amplicons) as described herein or known methods. The amplification products (amplicons) may be immobilized within the matrix generally at the location of the nucleic acid being amplified, thereby creating a localized colony of amplicons. The amplification products (amplicons) may be immobilized within the matrix by steric factors. The amplification products (amplicons) may also be immobilized within the matrix by covalent or noncovalent bonding. In this manner, the amplification products (amplicons) may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products (amplicons) are resistant to movement or unraveling under mechanical stress.

In some aspects, the amplification products (amplicons), are then copolymerized and/or covalently attached to the surrounding matrix thereby preserving their spatial relationship and any information inherent thereto. For example, if the amplification products (amplicons) are those generated from DNA or RNA within a cell embedded in the matrix, the amplification products (amplicons) can also be functionalized to form covalent attachment to the matrix preserving their spatial information within the cell thereby providing a subcellular localization distribution pattern.

In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described includes covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are included in the amplification step (e.g., RCA), functionalized with an acrylamide moiety using acrylic acid N-hydroxysuccinimide esters, and copolymerized with acrylamide monomers to form a hydrogel.

As used herein, the terms “hydrogel” or “hydrogel network” mean a network of polymer chains that are water-insoluble, sometimes found as a colloidal gel in which water is the dispersion medium. For example, hydrogels are a class of polymeric materials that can absorb large amounts of water without dissolving. In some aspects, hydrogels can contain over 99% water and may include natural or synthetic polymers, or a combination thereof.

In some aspects, hydrogels also possess a degree of flexibility very similar to natural tissue, due to their significant water content. Exemplary of suitable hydrogels include those described in, for example, US 2010/0055733. In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference. A “hydrogel” as described herein can include a cross-linked 3D network of hydrophilic polymer chains. As used herein, the terms “hydrogel subunits” or “hydrogel precursors” mean hydrophilic monomers, prepolymers, or polymers that can be cross-linked, or “polymerized”, to form a three-dimensional (3D) hydrogel network. Without being bound by theory, this fixation of the biological specimen in the presence of hydrogel subunits can cross-link the components of the specimen to the hydrogel subunits, thereby securing molecular components in place, preserving the tissue architecture and cell morphology. In some embodiments, the embedding includes copolymerizing the one or more amplification products with acrylamide. As used herein, the term “copolymer” describes a polymer which contains more than one type of subunit. The term encompasses polymer which include two, three, four, five, or six types of subunits.

A hydrogel can swell in the presence of water. In some embodiments, a hydrogel comprises a natural material. In some embodiments, a hydrogel includes a synthetic material. In some embodiments, a hydrogel includes a hybrid material, e.g., the hydrogel material comprises elements of both synthetic and natural polymers. Any of the materials used in hydrogels or hydrogels comprising a polypeptide-based material described herein can be used. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other known hydrogel-formation methods. For example, the biological sample can be immobilized in the hydrogel by polyacrylamide cross-linking. Further, analytes of a biological sample can be immobilized in a hydrogel by cross-linking (e.g., polyacrylamide cross-linking).

The composition and application of the hydrogel to a biological sample typically depends on the nature and preparation of the biological sample (e.g., sectioned, non-sectioned, fresh-frozen tissue, type of fixation). A hydrogel can be any appropriate hydrogel where upon formation of the hydrogel on the biological sample the biological sample becomes anchored to or embedded in the hydrogel. Non-limiting examples of hydrogels are described herein or are known. As one example, where the biological sample is a tissue section, the hydrogel can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel are formed in compartments, including but not limited to devices used to culture, maintain, or transport the cells. For example, hydrogels can be formed with monomer solution plus APS/TEMED added to the compartment to a depth ranging from about 0.1 μm to about 5 mm.

In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE) (available from ThermoFisher, Waltham, Mass.), Label-IT Amine (available from MirusBio, Madison, Wis.) and Label X).

In some embodiments, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., capture probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell tagging agent is added to the hydrogel before, contemporaneously with, and/or after polymerization. In some embodiments, a cell-penetrating agent is added to the hydrogel before, contemporaneously with, and/or after polymerization.

In some embodiments, a biological sample is embedded in a hydrogel to facilitate analysis of the sample, e.g., analysis of the target nucleic acids in the sample. In some embodiments, a biological sample on a substrate can be covered with any of the prepolymer solutions described herein. In some embodiments, the prepolymer solution can be polymerized such that a hydrogel is formed on top of and/or around the biological sample. Hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate (e.g., glass slide) results in the biological sample separating from the substrate along with the hydrogel.

In some embodiments, the hydrogel is removed after contacting the biological sample with the spatial array. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored in a medium before or after clearing of hydrogel (e.g., a mounting medium, methylcellulose, or other semi-solid mediums).

In some embodiments, the hydrogel chemistry can be tuned to specifically bind (e.g., retain) particular species of analytes (e.g., RNA, DNA, protein, etc.). In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE), Label-IT Amine and Label X. Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

Additional methods and aspects of hydrogel embedding of biological samples are described for example in Chen et al., Science 347(6221):543-548, 2015, the entire contents of which are incorporated herein by reference.

In some aspects, the embedding includes clearing the one or more hydrogel-embedded amplification products wherein the target nucleic acid is substantially retained in the one or more hydrogel-embedded amplification products. In some embodiments, the clearing includes substantially removing a plurality of cellular components from the one or more hydrogel-embedded amplification products. In some other embodiments, the clearing includes substantially removing lipids from the one or more hydrogel-embedded amplification products. As used herein, the term “substantially” means that the original amount present in the sample before clearing has been reduced by approximately 70% or more, such as by 75% or more, such as by 80% or more, such as by 85% or more, such as by 90% or more, such as by 95% or more, such as by 99% or more, such as by 100%.

In some embodiments, clearing the hydrogel-embedded amplification products includes electrophoresing the specimen. In some embodiments, the amplification products are electrophoresed using a buffer solution that includes an ionic surfactant. In some embodiments, the ionic surfactant is sodium dodecyl sulfate (SDS). In some embodiments, the specimen is electrophoresed using a voltage ranging from about 10 to about 60 volts. In some embodiments, the specimen is electrophoresed for a period of time ranging from about 15 minutes up to about 10 days. In some embodiments, the methods further involve incubating the cleared specimen in a mounting medium that has a refractive index that matches that of the cleared tissue. In some embodiments, the mounting medium increases the optical clarity of the specimen. In some embodiments, the mounting medium includes glycerol.

In some aspects, the matrix comprises one or more nucleic acids, such as one or more oligonucleotides. In some aspects, the polynucleotide probe sets and/or amplification products comprise one or more hybridization site(s) and the hybridization site(s) on the polynucleotide probes may hybridize to the nucleic acid matrix.

In some aspects, a matrix is used in conjunction with a solid support. For example the matrix can be polymerized in such a way that one surface of the matrix is attached to a solid support (e.g., a glass surface), while the other surface of the matrix is exposed or sandwiched between two solid supports. According to one aspect, the matrix can be contained within a container.

In some aspects, solid supports can be fashioned into a variety of shapes. In some embodiments, the solid support is substantially planar. Examples of solid supports include plates such as slides, microtitre plates, flow cells, coverslips, microchips, and the like, containers such as microfuge tubes, test tubes and the like, tubing, sheets, pads, films and the like. Additionally, the solid supports may be, for example, biological, nonbiological, organic, inorganic, or a combination thereof.

In some aspects, the methods involve producing a three dimensional nucleic acid amplicon matrix which is stable, long-lasting and resistant, substantially resistant or partially resistant to enzymatic or chemical degradation. The three dimensional nucleic acid amplicon matrix can be repeatedly interrogated using standard probe hybridization and/or fluorescence based sequencing. The three dimensional nucleic acid amplicon matrix can be repeatedly interrogated with little or no signal degradation, such as after more than 50 cycles, and with little position shift, such as less than 1 μm per amplicon.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### VI. COMPOSITIONS, KITS, DEVICES AND SYSTEMS

Also provided herein are compositions, kits, devices and systems to be used in accordance with the provided methods. Provided in some aspects are kits that include any first polynucleotide, second polynucleotide, and third polynucleotide as described herein. In some aspects, provided are polynucleotide probe sets, such as polynucleotide probe sets comprising a first polynucleotide, a second polynucleotide and a third polynucleotide as described herein. In some aspects, provided are compositions comprising one or more of the polynucleotides of a polynucleotide probe set comprising a first polynucleotide, a second polynucleotide and a third polynucleotide as described herein. In some aspects, also provided are kits comprising such polynucleotide probe sets and other components for carrying out the described methods.

In some aspects, provided are compositions comprising one or more polynucleotide(s) of the polynucleotide probe set. In some aspects, the composition comprises the first polynucleotide as described herein, for example, in Section III.A. In some aspects, the composition comprises the second polynucleotide as described herein, for example, in Section III.B. In some aspects, the composition comprises the third polynucleotide as described herein, for example, in Section III.C.

Also provided are kits, for example, comprising one or more polynucleotide(s) of the polynucleotide probe set and reagents for performing the methods provided herein, for example, reagents required for one or more steps including hybridization, ligation, amplification, detection, sequencing, sample preparation, embedding and/or anchoring as described herein. In some embodiments, the kit further comprises a target nucleic acid, e.g., any described in Sections III or V. In some embodiments, any or all of the polynucleotides are DNA molecules. In some embodiments, the target nucleic acid is a messenger RNA molecule. In some embodiments, the kit further comprises a ligase, for instance for forming a ligated circular probe from the padlock probe. In some embodiments, the ligase has DNA-splinted DNA ligase activity. In some embodiments, the kit further comprises a polymerase, for instance for performing amplification of the padlock probe. In some embodiments, the polymerase is capable of using the anchor as a primer and the circular probe as a template for amplification, e.g., using any of the methods described in Section III. In some embodiments, the kit further comprises a primer for amplification. In some aspects, the kit contains the matrix or material for forming the matrix, such as any described herein, for example, in Section V.D. In some aspects, the kit contains the reagents for detecting or analysis of any of the sequences described herein. In some embodiments, the kit further includes one or more components for performing a hybridization chain reaction (HCR), a linear oligonucleotide hybridization chain reaction (LO-HCR), a primer exchange reaction (PER), or for assembly of branched structures. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for embedding the biological sample. In some embodiments, the kits contain reagents, such as enzymes and buffers for ligation and/or amplification, such as ligases and/or polymerases. In some aspects, the kit can also include any of the reagents described herein, e.g., imaging buffer, wash buffer, strip buffer, ligation buffer and staining solutions. In some embodiments, the kits contain reagents for detection and/or sequencing, such as detection probes or detectable labels. In some embodiments, the kits optionally contain other components, for example: nucleic acid primers, enzymes and reagents, buffers, nucleotides, modified nucleotides, reagents for additional assays.

Also provided are devices for performing aspects of the described embodiments. Exemplary devices may include, for example, imaging chambers, electrophoresis apparatus, flow chambers, microscopes, needles, tubing, pumps. In some aspects, also provided are systems for performing the described embodiments. In some aspects, systems may include one or more of the modules described herein, e.g., a power supply, a refrigeration unit, waste, a heating unit, a pump, etc. Systems in accordance with certain embodiments may also include a microscope and/or related imaging equipment, e.g., camera components, digital imaging components and/or image capturing equipment and/or computer processors configured to collect images according to one or more user inputs.

In some aspects, provided systems can also include a fluidics device having an imaging chamber and a pump; and a processor unit configured to perform the methods for in situ target nucleic acid analysis in a cell in an intact tissue as described herein. In some embodiments, the system enables the automation of certain steps of the provided methods, for example, including repeated rounds of hybridization of detection probes, contacting with detection probes, washing off the excess detection probes, imaging, and stripping off the detection probes for the next round of sequencing. In some embodiments, the system may allow for continual operation. In some embodiments, the system includes an imaging chamber for flowing sequencing chemicals involved in in situ target nucleic acid analysis over a sample. In some embodiments, the system of fluidics and pumps control sequencing chemical delivery to the sample.

In some aspects, buffers may be added/removed/recirculated/replaced by the use of the one or more ports and optionally, tubing, pumps, valves, or any other suitable fluid handling and/or fluid manipulation equipment, for example, tubing that is removably attached or permanently attached to one or more components of a device. In some embodiments, the system includes a non-transitory computer-readable storage medium that has instructions, which when executed by the processor unit, cause the processor unit to control the delivery of chemicals and synchronize this process with a microscope. In some embodiments, the non-transitory computer-readable storage medium includes instructions, which when executed by the processor unit, cause the processor unit to measure an optical signal.

### VII. APPLICATIONS

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect particular sequences of interest in target nucleic acids.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. The term “polypeptide” can also include fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” can also include polypeptides including one or more of a fatty acid moiety, a lipid moiety, a sugar moiety, and a carbohydrate moiety.

As used herein, the term “target nucleic acid” is any polynucleotide nucleic acid molecule (e.g., DNA molecule; RNA molecule, modified nucleic acid, etc.) for assessment in accordance with the provided embodiments, such as a polynucleotide present in a cell. In some embodiments, the target nucleic acid is a coding RNA (e.g., mRNA). The target may, In some embodiments, be a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, i.e., have a differing abundance, within a cell population, wherein the methods of the invention allow profiling and comparison of the expression levels of nucleic acids, including but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. The backbone of the polynucleotide can include sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used herein, a composition refers to any mixture of two or more products, substances, or compounds, including cells. It may be a solution, a suspension, liquid, powder, a paste, aqueous, non-aqueous or any combination thereof.

### IX. EXAMPLES

The following examples are included for illustrative purposes only and are not intended to limit the scope of the invention.

### Example 1: Design of Probes

This Example describes the design of a three-polynucleotide probe set for amplification and sequencing of target nucleic acid sequences, such as messenger RNA (mRNA) sequences. The probe set permits highly sensitive and specific detection and sequencing of nucleic acid sequences in highly complex samples, for example, in intact biological tissue containing numerous different mRNA sequences, allowing image-based quantification of nucleotide expression at subcellular resolution. The three-polynucleotide probe set provides advantages of high specificity, as all three polynucleotides must be in proximity and hybridize to the target polynucleotide, for amplification and detection to occur. In addition, the exemplary method uncouples (i) the ligation step for circularizing a probe polynucleotide for amplification, and (ii) amplification priming (e.g., by Phi29), further increasing specificity. The probe set also provides additional spaces for barcoding, and the amplification products can be anchored or cross-linked to a matrix or nucleic acids. Further, in view of the location of the hybridization region in the amplified probe and the direction of synthesis, the probe set can minimize the binding or amplification of incomplete probes that can reduce the signal.

FIGS. 1 and 2 depict schematics of an exemplary probe set. The probe set includes three polynucleotides: a first polynucleotide (also called “left probe”), a second polynucleotide (also called “center probe” or “padlock probe”) and a third polynucleotide (also called “right probe”). Each of the polynucleotides in the probe set includes two or more hybridization regions (HRs), such as regions that are complementary or sufficiently complementary to a different nucleic acid sequence (for example, to the target nucleic acid, such as an mRNA, or to another HR present in a polynucleotide of the probe set) to form complexes via Watson-Crick base pairing.

The first polynucleotide (e.g., left probe) contains, in the 5′ to 3′ direction, hybridization regions HR1 and HRa′, with reference to the HR regions as depicted in FIGS. 1 and 2. HR1 of the polynucleotide can hybridize to (i.e., is complementary to or has sufficient complementarity for hybridization) HR1′ present on the target nucleic acid (e.g., mRNA). HRa′ of the first polynucleotide can hybridize to HRa of the second polynucleotide. The first polynucleotide can also be modified, for example at the 5′ end (depicted as a diamond in FIG. 2), to allow anchoring to a scaffold or a matrix, such as to a hydrogel. The first polynucleotide can also contain a barcode sequence, for example, at or near the 5′ end (depicted as a hashed rectangles in FIG. 2). The 3′ end sequences (e.g., HRa′) and the 3′-OH group of the first polynucleotide is used as a primer for rolling circle amplification of the second polynucleotide (“RCA primer” in FIG. 2).

The second polynucleotide (e.g., center probe) contains, in the 5′ to 3′ direction, hybridization regions HRb1, HRa, HR2, and HRb2. The second polynucleotide can also contain a barcode sequence, such as a gene-specific barcode (depicted as a filled rectangle in FIG. 2), for example, between the 5′ end and the 3′ end, such as between the 5′ end and HRa. HRa of the second polynucleotide can hybridize to HRa′ of the first polynucleotide. HRb1 at the 5′ end and HRb2 at the 3′ end of the second polynucleotide together can hybridize to HRb′ of the third polynucleotide, by split hybridization, with HRb′ acting as a splint to align the 5′ and 3′ ends of the second polynucleotide in place prior to ligation (in some cases including gap-filling). After split hybridization and ligation of the 5′ and 3′ ends of the second polynucleotide to circularize the second polynucleotide, the second polynucleotide can be amplified via rolling circle amplification, using the 3′ end of the first polynucleotide as a primer (indicated as “RCA” in FIG. 2).

The third polynucleotide (e.g., right probe) contains, in the 5′ to 3′ direction, hybridization regions HRb′ and HR3. HRb of the third polynucleotide can hybridize to HRb1 at the 5′ end and HRb2 at the 3′ end of the second polynucleotide by split hybridization, with HRb′ acting as a splint. In some cases, the third polynucleotide can also contain a barcode sequence and/or can be modified (for example, indicated as “barcode and/or modification” in FIG. 2), at or near the 3′ end.

The three polynucleotide probes can each hybridize to a region in the target nucleic acid (e.g., mRNA) via different target sites in the target nucleic acid (containing hybridization regions that are complementary to or sufficiently complementary to target hybridization regions in the polynucleotide probes). The target nucleic acid contains target sites HR1′, HR2′, and HR3′, which can hybridize to HR1 of the first polynucleotide, HR2 of the second polynucleotide, and HR3 of the third polynucleotide, respectively. The HR1′, HR2′ and HR3′ regions are designated in sequential configuration, in the order of HR1′, HR2′ and HR3′ in the 3′ to 5′ direction of the target nucleic acid. The HR1, HR2 and HR3 regions in the first polynucleotide, the second polynucleotide and the third polynucleotide is complementary to, or is sufficiently complementary to the HR1, HR2 and HR3, respectively, of the target nucleic acid. As depicted in FIGS. 1 and 2, upon hybridization, the first polynucleotide (e.g., left probe), the second polynucleotide (e.g., center probe) and the third polynucleotide (e.g., right probe) will hybridize in a sequential configuration, in the 3′ to 5′ direction of the target nucleic acid. Each of HR1, HR2 and HR3 can independently about 4 to about 16 nucleotides in length, such as about 8 to about 12 nucleotides in length, for example, about 10 nucleotides in length.

HR1′, HR2′ and HR3′ sites for a particular mRNA transcript can be selected as follows: a computational software is used to design the target sites (HR1′, HR2′ and HR3′ sites) for each polynucleotide probe set with length restriction of approximately 12-90 nucleotides; 1-10 sequences for each target nucleic acid (e.g., target mRNA) are designated; the resulting DNA sequences (12-90 nt) are split into three hybridization region sequences of 4-30 nt, with a 0-2 nt gap in between, and with the best match of melting temperature (Tₘ) between HR1, HR2 and HR3.

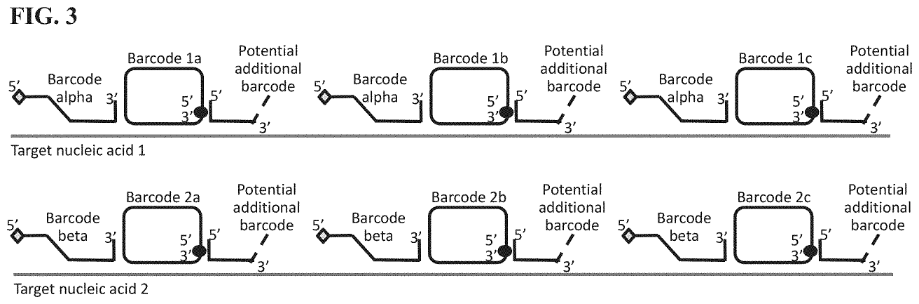

The polynucleotide probe set can contain one or more barcode(s) at various positions (for example, as depicted in FIG. 2 with solid or hashed rectangles. In some instances, the barcode present on the second polynucleotide can be used as a barcode for detection and/or sequencing, as the second polynucleotide is amplified, and therefore the barcode sequence is also amplified. In some instances, the optional additional barcodes can be present on the first polynucleotide and/or the third polynucleotide (hashed rectangles in FIG. 2). In some cases, these barcodes can provide additional barcoding space, for example for gene identification, functionalization, targeting of additional components, confirmation, error reduction and additional detection. For example, as shown in the schematic FIG. 3, one of the additional barcodes, for example on the first polynucleotide, can be used to identify all polynucleotide probe sets that hybridize to a particular target nucleic acid, for example, at different positions within the target nucleic acid.

### Example 2: Hybridization, Amplification and Detection

Exemplary probe sets as described in Example 1 above are hybridized, amplified and detected in an exemplary sample for in situ transcriptional profiling of cells in cell culture or a biological sample, such as a tissue section.

A library of different probe sets targeting different target nucleic acids, or different probe sets targeting the same nucleic acid at different positions, are pooled. The probe mixture is heated, then cooled down to room temperature, and incubated with a cell culture sample or a thin tissue section sample and hybridization buffer, for hybridization to target nucleic acids in the sample. Hybridization occurs at a temperature that is between the melting temperature (Tₘ) of the hybridization between HR1/HR1′, HR2/HR2′ or HR3/HR3′, and the Tₘ of the hybridization between HRa/HRa′, HRb1/HRb′ or HRb2/HRb′. The sample is washed, then incubated with a T4 DNA ligase for ligation of the 5′ and 3′ end of the second polynucleotide at room temperature. The sample is washed then incubated with a rolling-circle amplification (RCA) mixture containing a Phi29 DNA polymerase and dNTP, and incubated at approximately 30° C. for RCA of the second polynucleotide. In some instances, modified nucleotide bases (e.g., 5-(3-aminoallyl)-dUTP) is also included in the RCA mixture. After hybridization, ligation and amplification, the probe set, RCA amplification product (also called “amplicon”) and in some instances the target nucleic acid, can form a complex or a structure, such as a nanometer scale nucleic acid ball (also called “nanoball”).

For single-gene detection, fluorescently labeled oligonucleotides complementary to a portion of the amplified second polynucleotide (padlock probe) are incubated with the sample, washed and images are obtained.

For sequencing, the sample is treated with stripping buffer, washed, and incubated with a sequencing mixture containing a T4 DNA ligase, fluorescently labeled sequencing oligonucleotides, and images are obtained. Multiple cycles of sequencing is performed, and DAPI and Nissl staining can also be performed. Images are acquired using a confocal microscope.

### Example 3: Anchoring of Amplification Products

For three-dimensional analysis of target nucleic acids, the amplification products (amplicons or nanoballs) probe can be anchored in place to a scaffolding, cellular structures or other amplification products, to preserve the spatial information, increase mechanical stability of analytes, increase optical transparency and to increase permeability, for example, for reagents and components for detection. Various components of the three-polynucleotide probe set and amplification product can be exploited for functional groups for anchoring or cross-linking.

In one example the samples are embedded with scaffolding with polymers or matrices such as hydrogels, and the amplification products can be anchored to the polymer scaffolds or matrices via anchoring sites or hybridization sites. An exemplary schematic is in FIG. 4.

For hydrogel embedding, the cell culture or tissue section sample can be embedded before or after the ligation and amplification described in Example 2 above. In some examples, for cell culture or thin tissue section samples, after ligation and RCA, the sample can be washed and incubated in series with: acrylic acid NHC ester; a buffer containing acrylamide and bis-acrylamide monomers; and a mixture containing ammonium persulfate and tetramethylethylenediamine dissolved in the buffer. After embedding, the tissue-gel hybrid are digested with proteinases and washed. In some examples, thick tissue section samples can be embedded prior to ligation and RCA. After hybridization with the probe set, the sample can be washed and incubated in series with: acrylic acid NHC ester; a buffer containing acrylamide and bis-acrylamide monomers; and a mixture containing ammonium persulfate and tetramethylethylenediamine dissolved in the buffer. After embedding, the tissue-gel hybrid are digested with proteinases and washed.

The first polynucleotide (e.g., left probe) can be modified, for example at the 5′ end, to contain functional groups (for example, depicted as a diamond in FIG. 2) that can react with the scaffold to anchor the probe set, amplification product and the target nucleic acid to the scaffold, to cellular structures or to other amplification products. For anchoring to the hydrogel scaffold, the first polynucleotide can contain a modification containing a functional group that can react with or be incorporated into the acrylamide.

The second polynucleotide (padlock probe) can be modified to contain functional groups for anchoring or cross-linking to a scaffold, to cellular structures or to other amplification products. In some instances, during RCA, modified nucleotides containing functional groups that can react with or be incorporated into the scaffold, other cellular structures or other amplification products can be introduced for amplifying the second polynucleotide (for example, depicted as diamonds in FIG. 2). For example, the modified nucleotide 5-(3-aminoallyl)-dUTP can be introduced at low levels can be incorporated instead of dTTP during RCA. 5-(3-aminoallyl)-dUTP allows further functionalization with the polymerizable acrylamide moiety using acrylic acid N-hydroxysuccinimide ester (AA-NHS), such that the amplification products (amplified second polynucleotide) can be covalently anchored within the polyacrylamide network when embedding the sample with polyacrylamide hydrogels.

The third polynucleotide (e.g., right probe) can be modified, for example at the 3′ end (for example, depicted as stripe filled diamonds in FIG. 2), to contain functional groups that can react with or can be incorporated into the scaffold to anchor the probe set, amplification product and the target nucleic acid to the scaffold, to cellular structures or to other amplification products. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The probe set and amplification products generated from RCA (e.g., nanoball) can also be anchored or cross-linked to another probe set and/or amplification product (e.g., nanoballs). For example, in a nanoball, the amplification product (e.g., second polynucleotide or padlock probe) or the one or more of the probe polynucleotide can contain one or more functional groups at various different locations as described above, for example at the 5′ end of the first polynucleotide, within the amplified second polynucleotide as modified nucleotides and/or 3′ end of the third polynucleotide. Such functional groups can be exploited to anchor or cross-link the nanoball to other nanoballs, by direct cross-linking, or using symmetric or asymmetric bifunctional cross-linkers. Cross-linkers can be used to anchor or cross-link different nanoballs at a distance, or to other cellular structures or scaffolding. Examples of functional groups for anchoring the amplification product to the scaffold, to cellular structures or to other amplification products, include any described herein or known. An exemplary schematic is depicted in FIG. 5.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for analyzing a target nucleic acid, the method comprising:
contacting a target nucleic acid with a first polynucleotide, a second polynucleotide, and a third polynucleotide to form a hybridization complex, wherein:
the first polynucleotide comprises, in the 5′ to 3′ direction, hybridization regions HR1 and HRa′,
the second polynucleotide comprises, in the 5′ to 3′ direction, hybridization regions HRb1, HRa, HR2, and HRb2,
the third polynucleotide comprises, in the 5′ to 3′ direction, hybridization regions HRb′ and HR3,
the target nucleic acid comprises, in the 3′ to 5′ direction, hybridization regions HR1′, HR2′, and HR3′,
HR1, HR2, and HR3 hybridize to HR1′, HR2′, and HR3′, respectively,
HRa′ hybridizes to HRa, and
HRb1 and HRb2 form a split hybridization region that hybridizes to HRb′;
wherein HRb1 and HRb2 are connected using HRb′ as a splint to circularize the second polynucleotide;
wherein an amplification product is formed using the circularized second polynucleotide as a template and the first polynucleotide as a primer; and
wherein a sequence in the amplification product is analyzed, and the sequence is indicative of the target nucleic acid or a sequence thereof.

2. The method of claim 1, wherein the method further comprises:
circularizing the second polynucleotide by connecting HRb1 and HRb2 using HRb′ as a splint;
forming an amplification product using the circularized second polynucleotide as a template and the first polynucleotide as a primer; and/or
analyzing a sequence in the amplification product.

3.-38. (canceled)

39. The method of claim 1, wherein:
the split hybridization region formed by HRb1 and HRb2 comprises a nick between the 5′ end of HRb1 and the 3′ end of HRb2 when the split hybridization region is hybridized to HRb′, and wherein the method further comprises, without gap filling, ligating HRb1 and HRb2 using HRb′ as a splint; or
the split hybridization region formed by HRb1 and HRb2 comprises a gap between the 5′ end of HRb1 and the 3′ end of HRb2 when the split hybridization region is hybridized to HRb′, and wherein the method further comprises filling the gap and ligating HRb1 and HRb2 using HRb′ as a splint.

40.-47. (canceled)

48. The method of claim 1, wherein the target nucleic acid is an mRNA.

49. The method of claim 1, wherein:
the second polynucleotide comprises one or more barcode sequences BC1, BC2, . . . , and BCn, wherein n is an integer of 1 or greater;
the first polynucleotide comprises one or more barcode sequences BCα1, BCα2, . . . , and BCαp, wherein p is an integer of 1 or greater; and/or
the third polynucleotide comprises one or more barcode sequences BCa1, BCa2, . . . , and BCaq, wherein q is an integer of 1 or greater.

50. (canceled)

51. (canceled)

52. The method of claim 49, wherein at least one of BC1, BC2, . . . , and BCn, at least one of BCα1, BCα2, . . . , and BCαp, and/or at least one of BCa1, BCa2, . . . , and BCaq identifies the target nucleic acid or a sequence thereof, a unique identifier of a gene, or an error-checking barcode.

53. (canceled)

54. (canceled)

55. The method of claim 49, wherein the target nucleic acid is an mRNA and at least one of BC1, BC2, . . . , and BCn, at least one of BCα1, BCα2, . . . , and BCαp, and/or at least one of BCa1, BCa2, . . . , and BCaq identifies the mRNA as a splice variant, a transcriptional variant, and/or identify a splice junction sequence.

56. (canceled)

57. (canceled)

58. The method of claim 1, wherein the first polynucleotide, the second polynucleotide, and/or the third polynucleotide comprises an identifying sequence that identifies the target nucleic acid or a sequence thereof, wherein the identifying sequence is between about 3 and about 6 nucleotides in length and is in HRa, HRb1, and/or HRb2.

59. (canceled)

60. (canceled)

61. The method of claim 1, wherein:
the melting temperature (Tₘ) of HR1/HR1′ hybridization, the Tₘ of HR2/HR2′ hybridization, and/or the Tₘ of HR3/HR3′ hybridization are between about 40° C. and about 70° C., optionally about 60° C.;
the melting temperature (Tₘ) of HRa/HRa′ hybridization and/or the Tₘ of HRb1-HRb2/HRb′ hybridization are lower than the Tₘ of HR1/HR1′ hybridization, the Tₘ of HR2/HR2′ hybridization, and/or the Tₘ of HR3/HR3′ hybridization;
the melting temperature (Tₘ) of HRa/HRa′ hybridization and/or the Tₘ of HRb1-HRb2/HRb′ hybridization are lower than about 40° C. or is similar to or lower than room temperature; and/or
the hybridization complex is formed at a temperature higher than the melting temperature (Tₘ) of HRa/HRa′ hybridization and/or the Tₘ of HRb1-HRb2/HRb′ hybridization, but lower than the Tₘ of HR1/HR1′ hybridization, the Tₘ of HR2/HR2′ hybridization, and/or the Tₘ of HR3/HR3′ hybridization.

62.-67. (canceled)

68. The method of claim 1, wherein the circularization of the second polynucleotide comprises a ligation reaction.

69. (canceled)

70. The method of claim 68, wherein the ligation reaction is performed at a temperature lower than the temperature at which the hybridization complex is formed.

71.-75. (canceled)

76. The method of claim 1, wherein the amplification product is formed using rolling circle amplification (RCA).

77. (canceled)

78. (canceled)

79. The method of claim 1, wherein:
the amplification is performed at a temperature lower than the melting temperature (Tₘ) of HR1/HR1′ hybridization, the Tₘ of HR2/HR2′ hybridization, and/or the Tₘ of HR3/HR3′ hybridization; and/or
the amplification is performed at a temperature permissive to HRa/HRa′ hybridization and primer extension by a Phi29 polymerase.

80. (canceled)

81. (canceled)

82. The method of claim 2, wherein the analyzing the sequence is performed when the target nucleic acid and/or the amplification product is in situ in a tissue sample.

83.-86. (canceled)

87. The method of claim 82, wherein the tissue sample is embedded in a matrix.

88. (canceled)

89. (canceled)

90. The method of claim 2, wherein the analyzing the sequence comprises sequencing by hybridization, sequencing by ligation, and/or fluorescent in situ sequencing, and/or wherein the in situ hybridization comprises sequential fluorescent in situ hybridization.

91. (canceled)

92. The method of claim 2, wherein the analyzing the sequence comprises imaging the amplification product.

93. The method of claim 1, wherein the first polynucleotide, the second polynucleotide, and/or the third polynucleotide comprise an anchoring site comprising a functional group that is capable of reacting with a matrix.

94. A kit comprising a first polynucleotide, a second polynucleotide, and a third polynucleotide, wherein:
the first polynucleotide comprises, in the 5′ to 3′ direction, hybridization regions HR1 and HRa′,
the second polynucleotide comprises, in the 5′ to 3′ direction, hybridization regions HRb1, HRa, HR2, and HRb2,
the third polynucleotide comprises, in the 5′ to 3′ direction, hybridization regions HRb′ and HR3, and
the first polynucleotide, the second polynucleotide, and the third polynucleotide are capable of forming a hybridization complex with a target nucleic acid which comprises, in the 3′ to 5′ direction, hybridization regions HR1′, HR2′, and HR3′, wherein in the hybridization complex, HR1, HR2, and HR3 hybridize to HR1′, HR2′, and HR3′, respectively, HRa′ hybridizes to HRa, and HRb1 and HRb2 form a split hybridization region that hybridizes to HRb′.

95.-110. (canceled)

111. The kit of claim 94, wherein the first polynucleotide, the second polynucleotide, and/or the third polynucleotide comprise an anchoring site comprising a functional group that is capable of reacting with a matrix.

112.-115. (canceled)

116. A composition comprising a first polynucleotide, a second polynucleotide, a third polynucleotide, and a target nucleic acid, wherein:
the first polynucleotide comprises, in the 5′ to 3′ direction, hybridization regions HR1 and HRa′,
the second polynucleotide comprises, in the 5′ to 3′ direction, hybridization regions HRb1, HRa, HR2, and HRb2,
the third polynucleotide comprises, in the 5′ to 3′ direction, hybridization regions HRb′ and HR3, and
the first polynucleotide, the second polynucleotide, and the third polynucleotide forms a hybridization complex with the target nucleic acid which comprises, in the 3′ to 5′ direction, hybridization regions HR1′, HR2′, and HR3′, wherein in the hybridization complex, HR1, HR2, and HR3 hybridize to HR1′, HR2′, and HR3′, respectively, HRa′ hybridizes to HRa, and HRb1 and HRb2 form a split hybridization region that hybridizes to HRb′.

117. The composition of claim 116, wherein the second polynucleotide is circularized by connecting HRb1 and HRb2 using HRb′ as a splint.

118.-138. (canceled)

139. The method of claim 49, wherein the method comprises amplifying the one or more barcode sequences in situ, wherein the amplification in situ comprises a hybridization chain reaction (HCR) directly or indirectly on the one or more barcode sequences, linear oligonucleotide hybridization chain reaction (LO-HCR) directly or indirectly on the one or more barcode sequences, primer exchange reaction (PER) directly or indirectly on the one or more barcode sequences, assembly of branched structures directly or indirectly on the one or more barcode sequences, hybridization of a plurality of detectable probes directly or indirectly on the one or more barcode sequences, or any combination thereof.

140. (canceled)
